(19) United States
(12) Patent Application Publication
(10) Pub. No.: US 2022/0315965 A1
(43) Pub. Date: Oct. 6, 2022

(54) ENGINEERED BIOSYNTHETIC PATHWAYS FOR PRODUCTION OF CYSTATHIONINE BY FERMENTATION

(71) Applicant: Zymergen Inc., Emeryville, CA (US)

(72) Inventors: Cara Ann Tracewell, Walnut Creek, CA (US); Alexander Glennon Shearer, San Francisco, CA (US); Anupam Chowdhury, Emeryville, CA (US); Steven M. Edgar, Albany, CA (US)

(21) Appl. No.: 17/619,929

(22) PCT Filed: Jun. 24, 2020
(86) PCT No.: PCT/US2020/039469

§ 371 Date: Dec. 16, 2021

Related U.S. Application Data
(60) Provisional application No. 62/866,456, filed on Jun. 25, 2019.

(51) Int. Cl. C12P 13/12; C12N 15/75; C12N 15/77; C12N 15/81; C12N 9/02; C12N 9/10; C12N 9/12; C12N 9/88

(52) U.S. Cl. CPC C12P 13/12; C12N 15/75; C12N 15/77; C12N 15/815; C12N 9/0051; C12N 9/1085; C12N 9/1205; C12N 9/1241; C12N 9/88; C12Y 108/04008; C12Y 207/01025; C12Y 207/07004; C12Y 402/01022; C12Y 404/01001

(57) ABSTRACT

The present disclosure describes the engineering of microbial cells for fermentative production of cystathionine and provides novel engineered microbial cells and cultures, as well as related cystathionine production methods. An engineered microbial cell that expresses a heterologous cystathionine beta-synthase or a heterologous cystathionine gamma-synthase, wherein the engineered microbial cell produces cystathionine.

### CROSS-REFERENCE TO RELATED APPLICATIONS

This application claims the benefit of U.S. provisional application No. 62/866,456, filed Jun. 25, 2019, which is hereby incorporated by reference in its entirety.

### STATEMENT AS TO RIGHTS TO INVENTIONS MADE UNDER FEDERALLY SPONSORED RESEARCH AND DEVELOPMENT

This invention was made with Government support under Agreement No. HR0011-15-9-0014, awarded by DARPA. The Government has certain rights in the invention.

### INCORPORATION BY REFERENCE OF THE SEQUENCE LISTING

This application includes a sequence listing which has been submitted electronically in ASCII format and is hereby incorporated by reference in its entirety. This ASCII copy, created on Jun. 18, 2020, is named 2020-06-18_ZMGNP024WO_Seqlist_ST25.txt. and is 49,152 bytes in size.

### FIELD OF THE DISCLOSURE

The present disclosure relates generally to the area of engineering microbes for production of cystathionine by fermentation.

### BACKGROUND

Cystathionine is a di-amino acid containing an internal thioether bond. Recently, a deep-sea bacterium, Kocuria sp. 4 B has been described to produce a polymer containing 60-70% by mass of cystathionine. The polymer is reported to be biodegradable, and water-retentive and viscous when absorbing water. (See International Patent Publication No. WO2012133823, entitled “Novel useful deep-sea bacteria.”)

Cystathionine is produced from the amino acids serine and homoserine and a sulfur source such as sulfate or thiosulfate; it is a metabolic intermediate of the transsulfuration pathway between the sulfur-containing metabolites cysteine and homocysteine. (See FIG. 1.) The biosynthetic pathways for cysteine and homocysteine are part of the aspartate family of amino acids and have been studied in a number of organisms and show similarities as well as differences. Serine is produced in three steps from the glycolysis metabolite 3-phosphoglycerate. Homoserine is derived from the aspartate amino acid biosynthesis pathway. Either homoserine or serine is activated by acetylation (or succinylation) and sulfate (or thiosulfate) is incorporated by sulfhydrylation to produce cysteine or homocysteine.

There are two transsulfuration pathways in microorganisms: the “forward pathway” transfers a thiol group from cysteine to homocysteine and the “reverse pathway” transfers the thiol group from homocysteine to cysteine. The forward pathway occurs in two steps: first, cystathionine gamma-synthase catalyzes the γ-replacement of the acetyl (or succinyl group) in O-acetyl-L-homoserine (or O-succinyl-L-homoserine) with cysteine to produce cystathionine; and second, cystathionine beta-lyase cleaves cystathionine by means of β-elimination to produce homocysteine and an unstable imino acid, which is attacked by water to form pyruvate and ammonia. The reverse transsulfuration pathway also occurs in two steps: first, cystathionine beta-synthase catalyzes the reaction of serine with homocysteine to produce cystathionine; and second, cystathionine gamma-lyase cleaves cystathionine by means of γ-elimination to produce cysteine, alpha-ketobutyrate, and ammonia.

Cystathionine is a native metabolite in Saccharomyces cerevisiae, Yarrowia lipolytica, Corynebacteria glutamicum, and Bacillus subtillus; however, not all enzymes of the transsulfuration pathway or direct sulfhydrylation pathway are present in each of these hosts [1]. Therefore, cystathionine biosynthesis occurs via different routes in the native metabolism of these different hosts. A summary of cystathionine biosynthesis pathway genes native to the host organisms Saccharomyces cerevisiae, Corynebacterium glutamicum, Bacillus subtillus and Yarrowia lipolytica is given in the Table below.

| | | Yarrowia | Bacillus | Corynebacteria | Saccharomyces |
|---|---|---|---|---|---|
| | Enzyme activity | lipolytica | subtillus | glutamicum | cerevisiae |
| 1 | Cystathionine gamma-synthase | YALI0C22088p | yjcI | Cgl2446, | STR2 |
| | | | | Cgl2786 | |
| 2 | Cystathionine beta-synthase | YALI0E09108g | absent | absent | CYS4, NHS5, |
| | | | | | STR4, VMA41 |
| 3 | Cystathionine beta-lyase | YALI0D00605g | yjcJ | Cgl2309 | STR3, IRC7 |
| 4 | Cystathionine gamma-lyase | YALI0F05874g | yrhB | absent | CYS3 |
| 5 | Homocysteine synthase | | absent | Cgl0653 | MCY1, MET17, |
| | | | | | MET15, MET25 |
| 6 | 3-Phosphoglycerate dehydrogenase | + | + | + | + |
| 7 | Phosphoserine transaminase | + | + | + | + |
| 8 | Phosphoserine phosphatase | + | + | + | + |
| 9 | Serine O-acetyltransferase | absent | cysE | Cgl2563 | absent |
| 10 | Cysteine synthase | YALI0E08536p | cysK, ytkP | Cgl2562 | absent |
| 11 | Aspartate transaminase | + | + | + | + |
| 12 | Aspartate kinase | + | + | Cgl0251 | HOM3, BOR1, |
| | | | | | SIL4, THR3 |
| 13 | Aspartate-semialdehyde | + | + | Cgl0252 | HOM2, THR2 |
| | dehydrogenase | | | | |
| 14 | Homoserine dehydrogenase | + | + | Cgl1183 | HOM6, THR6 |
| 15 | Homoserine O-acetyltransferase | YALI0C24233g | absent | metX | MET2 |
| 16 | Homoserine O-succinyltransferase | + | + | + | + |
| 17 | Sulfur uptake | + | + | + | + |
| 18 | ATP sulfurylase | YALI0B08184p | sat | Cgl2814 | MET3 |
| 19 | APS kinase | YALI0E00418p | yisZ, cysC | not in KEGG | MET14 |
| 20 | PAPS reductase | YALI0B08140p | yitB | Cgl2816 | MET16 |
| 21 | Sulfite reductase | YALI0D11176p | yvgQ, cysJ | Cgl2817 | MET10 |
| 22 | Methionine synthase | + | + | + | + |

Saccharomyces cerevisiae only has the enzymes for converting homocysteine to cysteine [11]. Cystathionine intracellular accumulation in Saccharomyces cerevisiae has been reported resulting from loss of function mutations to cystathionine gamma-lyase (Cys3) [10]. Thus, in S. cerevisiae, cysteine biosynthesis occurs by sulfide incorporation into homoserine to form homocysteine, followed by conversion of homocysteine to cysteine thru the transsulfuration pathway. Although a pseudo cysteine synthase (sulfide incorporation to serine) has been annotated in the genome of S. cerevisiae, it has not been found to be functional [2].

In contrast, in Yarrowia lipolytica, both the forward and reverse transsulfuration pathway are present [3]. Thus, cysteine can be produced in Y. lipolytica by the O-acetyl-serine (OAS) pathway or direct sulfhydrylation pathway, as well as the reverse transsulfuration pathway. Y. lipolytica contains two genes that are orthologs of the S. cerevisiae gene pseudo-cysteine synthase gene, and these two genes encode cysteine synthases involved in the OAS pathway.

In Corynebacteria glutamicum, the transsulfuration pathway functions in the forward direction: cystathionine is made from L-cysteine and O-acetyl-L-homoserine by cystathionine gamma-synthase. Then, cystathionine is converted to L-homocysteine by cystathionine beta-lyase. Both cystathionine beta-synthase and cystathionine gamma-lyase activities are absent from C. glutamicum. Cystathionine gamma-synthase in C. glutamicum can use O-acetyl-L-homoserine (OAHS) or O-succinyl-L-homoserine (OSHS) with L-cysteine to produce cystathionine [4]. L-Cysteine is also made through direct sulfhydrylation of L-serine using sulfide by L-cysteine synthase [5], and L-homocysteine is made through direct sulfhydrylation of L-homoserine using sulfide and L-homocysteine synthase [6, 7].

In Bacillus subtillus, the transsulfuration pathway functions in the forward and reverse directions: L-cysteine can be converted to L-homocysteine by cystathionine gamma-synthase and cystathionine beta-lyase, and L-homocysteine can be converted to L-cysteine by cystathionine beta-synthase and cystathionine gamma-lyase. L-Cysteine is made through direct sulfhydrylation of L-serine using sulfide by L-cysteine synthase, but there is no homocysteine synthase activity that can use sulfide and L-homoserine to make homocysteine [9].

### SUMMARY

Production of sulfur-containing amino acid monomers such as cystathionine by biological fermentation can make the monomer economically accessible for a newly identified materials application. Sulfur-containing polymers have attractive hygroscopic and mechanical properties for novel material applications.

The disclosure provides engineered microbial cells, cultures of the microbial cells, and methods for the production of cystathionine, including the following:

Embodiment 1: An engineered microbial cell that expresses a heterologous cystathionine beta-synthase or a heterologous cystathionine gamma-synthase, wherein the engineered microbial cell produces cystathionine.

Embodiment 2: The engineered microbial cell of embodiment 1, wherein the engineered microbial cell expresses the heterologous cystathionine beta-synthase and the heterologous cystathionine gamma-synthase.

Embodiment 3: The engineered microbial cell of embodiment 1 or embodiment 2, wherein the engineered microbial cell includes increased activity of one or more upstream pathway enzyme(s), said increased activity being increased relative to a control cell.

Embodiment 4: The engineered microbial cell of embodiment 3, wherein the engineered microbial cell includes increased activity of one or more upstream pathway enzymes leading to cysteine.

Embodiment 5: The engineered microbial cell of embodiment 4, wherein the one or more upstream pathway enzymes leading to cysteine is/are selected from the group consisting of 3-phosphoglycerate dehydrogenase, phosphoserine transaminase, phosphoserine phosphatase, serine-O-acetyltransferase, and cysteine synthase.

Embodiment 6: The engineered microbial cell of any one of embodiments 3-5, wherein the engineered microbial cell includes increased activity of one or more upstream pathway enzymes leading to a homoserine.

Embodiment 7: The engineered microbial cell of embodiment 6, wherein the one or more upstream pathway enzymes leading to a homoserine is/are selected from the group consisting of phosphoenolpyruvate carboxylase, pyruvate carboxylase, malate dehydrogensase, aspartate transaminase (aspartate aminotransferase), aspartate kinase (aspartokinase), aspartate-semialdehyde dehydrogenase, homoserine dehydrogenase, L-homoserine-O-acetyltransferase, and L-homoserine-O-succinyltranferase (homoserine transsuccinylase).

Embodiment 8: The engineered microbial cell of embodiment 7, wherein the one or more upstream pathway enzymes leading to homoserine is/are selected from the group consisting of pyruvate carboxylase, aspartate transaminase, and aspartate kinase.

Embodiment 9: The engineered microbial cell of any one of embodiments 3-8, wherein the engineered microbial cell includes increased activity of one or more upstream pathway enzymes leading to homocysteine.

Embodiment 10: The engineered microbial cell of embodiment 9, wherein the one or more upstream pathway enzymes leading to homocysteine is/are selected from the group consisting of sulfate adenyltransferase (ATP sulfurylase), adenyl-sulfate kinase (APS kinase), phosphoadenosine phosphosulfate (PAPS) reductase, sulfite reductase, and homocysteine synthase.

Embodiment 11: The engineered microbial cell of embodiment 10, wherein the one or more upstream pathway enzymes leading to homocysteine includes sulfite reductase.

Embodiment 12: The engineered microbial cell of any one of embodiments 3-11, wherein the engineered microbial cell includes increased activity of one or more upstream pathway enzymes leading to serine.

Embodiment 13: The engineered microbial cell of embodiment 12, wherein the one or more upstream pathway enzymes leading to serine is/are selected from the group consisting of 3-phosphoglycerate dehydrogenase, phosphoserine transaminase, and phosphoserine phosphatase 14: The engineered microbial cell of any one of embodiments 1-13, wherein the activity of the one or more upstream pathway enzymes is increased by introducing one or more genes encoding the one or more upstream pathway enzymes.

Embodiment 15: The engineered microbial cell of embodiment 14, wherein at least two genes encoding the same enzyme are introduced.

Embodiment 16: The engineered microbial cell of any one of embodiments 3-15, wherein the activity of the one or more upstream pathway enzymes is increased by introducing one or more feedback-deregulated enzyme(s).

Embodiment 17: The engineered microbial cell of embodiment 16, where the one or more feedback-deregulated enzyme (s) is/are selected from the group consisting of a feedback-deregulated aspartate kinase, a feedback-deregulated homoserine dehydrogenase, a feedback-deregulated aspartate-semialdehyde dehydrogenase, a feedback-deregulated L-homoserine-O-succinyltranferase, a feedback-deregulated phoshoenolpyruvate carboxylase, and a feedback-deregulated pyruvate carboxylase.

Embodiment 18: The engineered microbial cell of embodiment 17, where the one or more feedback-deregulated enzyme(s) is/are selected from the group consisting of: (a) a feedback-deregulated Saccharomyces cerevisiae aspartate kinase (EC 2.7.2.4) including the amino acid substitution E250K or M318I; (b) a feedback-deregulated homoserine dehydrogenase (EC 1.1.1.3) including (i) the amino acid substitutions V104I, T116I, and G148A; or (ii) the amino acid substitutions A429L, K430S, P431L, V432L, V433L, K434R, A435Q, I436S, N437T, and S438V, and a deletion of amino acids 439-445; (c) a feedback-deregulated aspartate-semialdehyde dehydrogenase (EC 1.2.1.11) including the amino acid substitutions D66G, S202F, R234H, D272E, and K285E; (d) a feedback-deregulated L-homoserine-O-succinyltranferase (EC 2.3.1.46) including the amino acid substitution R27C or I296S; (e) a feedback-deregulated phosphoenol pyruvate carboxylase (EC 4.1.1.31) including the amino acid substitution N917G or D299N; and (f) a feedback-deregulated pyruvate carboxylase (EC 6.4.1.1) including the amino acid substitution P458S.

Embodiment 19: The engineered microbial cell of embodiment 18, wherein the one or more feedback-deregulated enzyme(s) comprise a feedback-deregulated Saccharomyces cerevisiae aspartate kinase (EC 2.7.2.4) including the amino acid substitution E250K or M318I.

Embodiment 20: The engineered microbial cell of any one of embodiments 1-19, wherein the engineered microbial cell includes reduced activity of one or more enzyme(s) that consume one or more upstream pathway precursors, said reduced activity being reduced relative to a control cell.

Embodiment 21: The engineered microbial cell of embodiment 20, wherein the one or more enzyme(s) that consume one or more upstream pathway precursors is/are selected from the group consisting of methionine synthase, homoserine kinase, threonine synthase, catabolic serine deaminase, glutathione synthase, and L-cysteine desulfhydrase.

Embodiment 22: The engineered microbial cell of any one of embodiments 1-21, wherein the engineered microbial cell includes reduced activity of one or more enzyme(s) that consume cystathionine, said reduced activity being reduced relative to a control cell.

Embodiment 23: The engineered microbial cell of embodiment 22, wherein the one or more enzyme(s) that consume cystathionine are selected from cystathionine beta-lyase and cystathionine gamma-lyase.

Embodiment 24: The engineered microbial cell of any one of embodiments 20-23, wherein the reduced activity is achieved by one or more means selected from the group consisting of gene deletion, gene disruption, altering regulation of a gene, and replacing a native promoter with a less active promoter.

Embodiment 25: The engineered microbial cell of any one of embodiments 1-24, wherein the engineered microbial cell includes increased activity of an amino acid exporter that is capable of exporting cystathionine, said increased activity being increased relative to a control cell.

Embodiment 26: The engineered microbial cell of any of embodiments 1-25, wherein the engineered microbial cell includes altered cofactor specificity of one or more upstream pathway enzyme(s) from the reduced form of nicotinamide adenine dinucleotide phosphate (NADPH) to the reduced from of nicotinamide adenine dinucleotide (NADH).

Embodiment 27: The engineered microbial cell of embodiment 26, wherein the one or more upstream pathway enzyme(s) whose cofactor specificity is altered is/are selected from the group consisting of aspartate semi-aldehyde dehydrogenase, homoserine dehydrogenase, and glyceraldehyde 3-phosphate dehydrogenase (GAPDH).

Embodiment 28: An engineered microbial cell that includes means for expressing a heterologous cystathionine beta-synthase or a heterologous cystathionine gamma-synthase, wherein the engineered microbial cell produces cystathionine.

Embodiment 29: The engineered microbial cell of embodiment 28, wherein the engineered microbial cell includes means for expressing the heterologous cystathionine beta-synthase and the heterologous cystathionine gamma-synthase.

Embodiment 30: The engineered microbial cell of any of embodiment 28 or embodiment 29, wherein the engineered microbial cell includes means for increasing the activity of one or more upstream pathway enzyme(s), said increased activity being increased relative to a control cell.

Embodiment 31: The engineered microbial cell of embodiment 30, wherein the engineered microbial cell includes means for increasing the activity of one or more upstream pathway enzymes leading to cysteine.

Embodiment 32: The engineered microbial cell of embodiment 31, wherein the one or more upstream pathway enzymes leading to cysteine is/are selected from the group consisting of 3-phosphoglycerate dehydrogenase, phosphoserine transaminase, phosphoserine phosphatase, serine-O-acetyltransferase, and cysteine synthase.

Embodiment 33: The engineered microbial cell of any one of embodiments 30-32, wherein the engineered microbial cell includes means for increasing the activity of one or more upstream pathway enzymes leading to a homoserine.

Embodiment 34: The engineered microbial cell of embodiment 33, wherein the one or more upstream pathway enzymes leading to a homoserine is/are selected from the group consisting of phosphoenolpyruvate carboxylase, pyruvate carboxylase, malate dehydrogensase, aspartate transaminase (aspartate aminotransferase), aspartate kinase (aspartokinase), aspartate-semialdehyde dehydrogenase, homoserine dehydrogenase, L-homoserine-O-acetyltransferase, and L-homoserine-O-succinyltranferase (homoserine transsuccinylase).

Embodiment 35: The engineered microbial cell of embodiment 34, wherein the one or more upstream pathway enzymes leading to a homoserine is/are selected from the group consisting of pyruvate carboxylase, aspartate transaminase, and aspartate kinase.

Embodiment 36: The engineered microbial cell of any one of embodiments 30-35, wherein the engineered microbial cell includes means for increasing the activity of one or more upstream pathway enzymes leading to homocysteine.

Embodiment 37: The engineered microbial cell of embodiment 36, wherein the one or more upstream pathway enzymes leading to homocysteine is/are selected from the group consisting of sulfate adenyltransferase (ATP sulfurylase), adenyl-sulfate kinase (APS kinase), phosphoadenosine phosphosulfate (PAPS) reductase, sulfite reductase, and homocysteine synthase.

Embodiment 38: The engineered microbial cell of embodiment 37, wherein the one or more upstream pathway enzymes leading to homocysteine includes sulfite reductase.

Embodiment 39: The engineered microbial cell of any one of embodiments 30-38, wherein the engineered microbial cell includes means for increasing the activity of one or more upstream pathway enzymes leading to serine.

Embodiment 40: The engineered microbial cell of embodiment 39, wherein the one or more upstream pathway enzymes leading to serine is/are selected from the group consisting of 3-phosphoglycerate dehydrogenase, phosphoserine transaminase, and phosphoserine phosphatase.

Embodiment 41: The engineered microbial cell of any one of embodiments 30-40, wherein the engineered microbial cell includes means for expressing one or more feedback-deregulated enzyme(s).

Embodiment 42: The engineered microbial cell of embodiment 41, where the one or more feedback-deregulated enzyme (s) is/are selected from the group consisting of a feedback-deregulated aspartate kinase, a feedback-deregulated homoserine dehydrogenase, a feedback-deregulated aspartate-semialdehyde dehydrogenase, a feedback-deregulated L-homoserine-O-succinyltranferase, a feedback-deregulated phoshoenolpyruvate carboxylase, and a feedback-deregulated pyruvate carboxylase.

Embodiment 43: The engineered microbial cell of any one of embodiments 28-42, wherein the engineered microbial cell includes means for reducing the activity of one or more enzyme(s) that consume one or more upstream pathway precursors, said reduced activity being reduced relative to a control cell.

Embodiment 44: The engineered microbial cell of embodiment 43, wherein the one or more enzyme(s) that consume one or more upstream pathway precursors is/are selected from the group consisting of methionine synthase, homoserine kinase, threonine synthase, catabolic serine deaminase, glutathione synthase, and L-cysteine desulfhydrase.

Embodiment 45: The engineered microbial cell of any one of embodiments 28-44, wherein the engineered microbial cell includes means for reducing the activity of one or more enzyme(s) that consume cystathionine, said reduced activity being reduced relative to a control cell.

Embodiment 46: The engineered microbial cell of embodiment 45, wherein the one or more enzyme(s) that consume cystathionine are selected from cystathionine beta-lyase and cystathionine gamma-lyase.

Embodiment 47: The engineered microbial cell of any one of embodiments 28-46, wherein the engineered microbial cell includes means for increasing the activity of an amino acid exporter that is capable of exporting cystathionine, said increased activity being increased relative to a control cell.

Embodiment 48: The engineered microbial cell of any of embodiments 28-47, wherein the engineered microbial cell includes means for altering the cofactor specificity of one or more upstream pathway enzyme(s) from the reduced form of nicotinamide adenine dinucleotide phosphate (NADPH) to prefer the reduced from of nicotinamide adenine dinucleotide (NADH).

Embodiment 49: The engineered microbial cell of embodiment 26, wherein the one or more upstream pathway enzyme(s) whose cofactor specificity is altered is/are selected from the group consisting of aspartate semi-aldehyde dehydrogenase, homoserine dehydrogenase, and glyceraldehyde 3-phosphate dehydrogenase (GAPDH).

Embodiment 50: The engineered microbial cell of any one of embodiments 1-49, wherein the engineered microbial cell is a bacterial cell.

Embodiment 51: The engineered microbial cell of embodiment 50, wherein the bacterial cell is a cell of the genus Corynebacteria.

Embodiment 52: The engineered microbial cell of embodiment 51, wherein the bacterial cell is a cell of the species glutamicum.

Embodiment 53: The engineered microbial cell of embodiment 52, wherein the engineered microbial cell includes a heterologous cystathionine beta-synthase having at least 70% amino acid sequence identity with a Saccharomyces cerevisiae cystathionine beta-synthase.

Embodiment 54: The engineered microbial cell of embodiment 53, wherein the engineered microbial cell additionally includes a heterologous cystathionine gamma-synthase having at least 70% amino acid sequence identity with an Escherichia coli cystathionine gamma-synthase.

Embodiment 55: The engineered microbial cell of embodiment 53 or embodiment 54, wherein the engineered microbial cell additionally includes a heterologous aspartate aminotransferase having at least 70% amino acid sequence identity with a Saccharomyces cerevisiae aspartate aminotransferase.

Embodiment 56: The engineered microbial cell of embodiment 50, wherein the bacterial cell is a cell of the genus Bacillus.

Embodiment 57: The engineered microbial cell of embodiment 56, wherein the bacterial cell is a cell of the species subtilis.

Embodiment 58: The engineered microbial cell of embodiment 57, wherein the engineered microbial cell includes a heterologous cystathionine beta-synthase having at least 70% amino acid sequence identity with a Saccharomyces cerevisiae cystathionine beta-synthase.

Embodiment 59: The engineered microbial cell of embodiment 58, wherein the engineered microbial cell additionally includes a heterologous cystathionine gamma-synthase having at least 70% amino acid sequence identity with a Bacillus paralicheniformis cystathionine gamma-synthase.

Embodiment 60: The engineered microbial cell of embodiment 58 or embodiment 59, wherein the engineered microbial cell additionally includes a feedback-deregulated aspartokinase having at least 70% amino acid sequence identity with a feedback-deregulated Saccharomyces cerevisiae aspartokinase.

Embodiment 61: The engineered microbial cell of any one of embodiments 1-49, wherein the engineered microbial cell includes a fungal cell.

Embodiment 62: The engineered microbial cell of embodiment 61, wherein the engineered microbial cell includes a yeast cell.

Embodiment 63: The engineered microbial cell of embodiment 62, wherein the yeast cell is a cell of the genus Saccharomyces.

Embodiment 64: The engineered microbial cell of embodiment 63, wherein the yeast cell is a cell of the species cerevisiae.

Embodiment 65: The engineered microbial cell of embodiment 64, wherein the engineered microbial cell includes a heterologous cystathionine beta-synthase having at least 70% amino acid sequence identity with a Saccharomyces cerevisiae cystathionine beta-synthase.

Embodiment 66: The engineered microbial cell of embodiment 65, wherein the engineered microbial cell additionally includes a heterologous cystathionine gamma-synthase having at least 70% amino acid sequence identity with an Escherichia coli cystathionine gamma-synthase.

Embodiment 67: The engineered microbial cell of embodiment 65 or 66, wherein the engineered microbial cell additionally includes a feedback-deregulated aspartokinase having at least 70% amino acid sequence identity with a feedback-deregulated Saccharomyces cerevisiae aspartokinase.

Embodiment 68: The engineered microbial cell of embodiment 62, wherein the yeast cell is a cell of the genus Yarrowia.

Embodiment 69: The engineered microbial cell of embodiment 68, wherein the yeast cell is a cell of the species lipolytica.

Embodiment 70: The engineered microbial cell of embodiment 69, wherein the engineered microbial cell includes a heterologous cystathionine beta-synthase having at least 70% amino acid sequence identity with a Saccharomyces cerevisiae cystathionine beta-synthase.

Embodiment 71: The engineered microbial cell of embodiment 70, wherein the engineered microbial cell additionally includes a heterologous cystathionine gamma-synthase having at least 70% amino acid sequence identity with a Bacillus paralicheniformis cystathionine gamma-synthase.

Embodiment 72: The engineered microbial cell of embodiment 70 or embodiment 71, wherein the engineered microbial cell additionally includes a feedback-deregulated aspartokinase having at least 70% amino acid sequence identity with a feedback-deregulated Saccharomyces cerevisiae aspartokinase.

Embodiment 73: The engineered microbial cell of any one of embodiments 1-72, wherein, when cultured, the engineered microbial cell produces cystathionine at a level at least 50 μg/L of culture medium.

Embodiment 74: The engineered microbial cell of embodiment 73, wherein, when cultured, the engineered microbial cell produces cystathionine at a level at least 1 mg/L of culture medium.

Embodiment 75: The engineered microbial cell of embodiment 74, wherein, when cultured, the engineered microbial cell produces cystathionine at a level at least 4 gm/L of culture medium.

Embodiment 76: A culture of engineered microbial cells according to any one of embodiments 1-75.

Embodiment 77: The culture of embodiment 76, wherein the substrate includes a carbon source and a nitrogen source selected from the group consisting of urea, an ammonium salt, ammonia, and any combination thereof.

Embodiment 78: The culture of embodiment 76 or embodiment 77, wherein the engineered microbial cells are present in a concentration such that the culture has an optical density at 600 nm of 10-500.

Embodiment 79: The culture of any one of embodiments 76-78, wherein the culture includes cystathionine.

Embodiment 80: The culture of any one of embodiments 76-79, wherein the culture includes cystathionine at a level at least 4 mg/L of culture medium.

Embodiment 81: A method of culturing engineered microbial cells according to any one of embodiments 1-75, the method including culturing the cells under conditions suitable for producing cystathionine.

Embodiment 82: The method of embodiment 81, wherein the method includes fed-batch culture, with an initial glucose level in the range of 1-100 g/L, followed controlled sugar feeding.

Embodiment 83: The method of embodiment 81 or embodiment 82, wherein the fermentation substrate includes glucose and a nitrogen source selected from the group consisting of urea, an ammonium salt, ammonia, and any combination thereof.

Embodiment 84: The method of any one of embodiments 81-83, wherein the culture is pH-controlled during culturing.

Embodiment 85: The method of any one of embodiments 81-84, wherein the culture is aerated during culturing.

Embodiment 86: The method of any one of embodiments 81-85, wherein the engineered microbial cells produce cystathionine at a level at least 4 mg/L of culture medium.

Embodiment 87: The method of any one of embodiments 81-86, wherein the method additionally includes recovering cystathionine from the culture.

Embodiment 88: A method for preparing cystathionine using microbial cells engineered to produce cystathionine, the method including: (a) expressing a heterologous cystathionine beta-synthase and/or a heterologous cystathionine gamma-synthase in microbial cells; (b) cultivating the microbial cells in a suitable culture medium under conditions that permit the microbial cells to produce cystathionine, wherein the cystathionine is released into the culture medium; and (c) isolating cystathionine from the culture medium.

### BRIEF DESCRIPTION OF THE DRAWINGS

FIG. 1: Biosynthetic pathways for cystathionine.

FIG. 2: Cystathionine titers measured in the extracellular broth following fermentation by first-round engineered host Corynebacteria glutamicum. (See also Example 1.)

FIG. 3: Cystathionine titers measured in the extracellular broth following fermentation by first-round engineered host Saccharomyces cerevisiae. (See also Example 1.)

FIG. 4: Cystathionine titers measured in the extracellular broth following fermentation by second-round engineered host S. cerevisiae. (See also Example 1.)

FIG. 5: Cystathionine titers measured in the extracellular broth following fermentation by third-round engineered host S. cerevisiae. (See also Example 1.)

FIG. 6: Cystathionine titers measured in the extracellular broth following fermentation by first-round engineered host Yarrowia lipolytica. (See also Example 1.)

FIG. 7: Cystathionine titers measured in the extracellular broth following fermentation by first-round engineered host Bacillus subtillus. (See also Example 1.)

FIG. 8: Cystathionine titers measured in the extracellular broth following fermentation by the host evaluation designs tested in S. cerevisiae.

FIG. 9: Cystathionine titers measured in the extracellular broth following fermentation by the host evaluation designs tested in C. glutamicum.

FIG. 10: Cystathionine titers measured in the extracellular broth following fermentation by fourth-round (improvement-round) engineered host S. cerevisiae.

FIG. 11: Integration of Promoter-Gene-Terminator into Saccharomyces cerevisiae and Yarrowia lipolytica.

FIG. 12: Promoter replacement in Saccharomyces cerevisiae and Yarrowia lipolytica.

FIG. 13: Targeted gene deletion in Saccharomyces cerevisiae and Yarrowia lipolytica.

FIG. 14: Integration of Promoter-Gene-Terminator into Corynebacteria glutamicum and Bacillus subtilis.

### DETAILED DESCRIPTION

This disclosure describes a method for the production of the small molecule, cystathionine, via fermentation by a microbial host from simple carbon and nitrogen sources, such as glucose and urea, respectively. This aim is achieved via enhancing the metabolic pathway(s) leading to cystathionine in a suitable microbial host for industrial fermentation of large-scale chemical products such as Saccharomyces cerevisiae, Corynebacteria glutamicum, Bacillus subtillus and Yarrowia lipolytica. In certain embodiments, the microbial host has enhanced biosynthesis of the amino acid precursors L-cysteine and L-homoserine and a highly active cysteine gamma-synthase.

Cysteine beta- or gamma-synthases active in S. cerevisiae have been identified, and additional strain modifications have been made to enable industrial-scale host production of cystathionine, including installation of cysteine synthase, feedback-deregulated homoserine dehydrogenase, feedback-deregulated aspartate kinase, constitutive expression of serine and homoserine pathway enzymes, and decreasing or eliminating activities of cystathionine gamma-lyase, cystathionine beta-lyase, and cysteine desulfurases.

### Definitions

Terms used in the claims and specification are defined as set forth below unless otherwise specified.

The term “fermentation” is used herein to refer to a process whereby a microbial cell converts one or more substrate(s) into a desired product (such as cystathionine) by means of one or more biological conversion steps, without the need for any chemical conversion step.

The term “engineered” is used herein, with reference to a cell, to indicate that the cell contains at least one targeted genetic alteration introduced by man that distinguishes the engineered cell from the naturally occurring cell.

The term “native” is used herein to refer to a cellular component, such as a polynucleotide or polypeptide, that is naturally present in a particular cell. A native polynucleotide or polypeptide is endogenous to the cell.

When used with reference to a polynucleotide or polypeptide, the term “non-native” refers to a polynucleotide or polypeptide that is not naturally present in a particular cell.

When used with reference to the context in which a gene is expressed, the term “non-native” refers to a gene expressed in any context other than the genomic and cellular context in which it is naturally expressed. A gene expressed in a non-native manner may have the same nucleotide sequence as the corresponding gene in a host cell, but may be expressed from a vector or from an integration point in the genome that differs from the locus of the native gene.

The term “heterologous” is used herein to describe a polynucleotide or polypeptide introduced into a host cell. This term encompasses a polynucleotide or polypeptide, respectively, derived from a different organism, species, or strain than that of the host cell. In this case, the heterologous polynucleotide or polypeptide has a sequence that is different from any sequence(s) found in the same host cell. However, the term also encompasses a polynucleotide or polypeptide that has a sequence that is the same as a sequence found in the host cell, wherein the polynucleotide or polypeptide is present in a different context than the native sequence (e.g., a heterologous polynucleotide can be linked to a different promotor and inserted into a different genomic location than that of the native sequence). “Heterologous expression” thus encompasses expression of a sequence that is non-native to the host cell, as well as expression of a sequence that is native to the host cell in a non-native context.

As used with reference to polynucleotides or polypeptides, the term “wild-type” refers to any polynucleotide having a nucleotide sequence, or polypeptide having an amino acid, sequence present in a polynucleotide or polypeptide from a naturally occurring organism, regardless of the source of the molecule; i.e., the term “wild-type” refers to sequence characteristics, regardless of whether the molecule is purified from a natural source; expressed recombinantly, followed by purification; or synthesized. The term “wild-type” is also used to denote naturally occurring cells.

A “control cell” is a cell that is otherwise identical to an engineered cell being tested, including being of the same genus and species as the engineered cell, but lacks the specific genetic modification(s) being tested in the engineered cell.

Enzymes are identified herein by the reactions they catalyze and, unless otherwise indicated, refer to any polypeptide capable of catalyzing the identified reaction. Unless otherwise indicated, enzymes may be derived from any organism and may have a native or mutated amino acid sequence. As is well known, enzymes may have multiple functions and/or multiple names, sometimes depending on the source organism from which they derive. The enzyme names used herein encompass orthologs, including enzymes that may have one or more additional functions or a different name.

The term “feedback-deregulated” is used herein with reference to an enzyme that is normally negatively regulated by a downstream product of the enzymatic pathway (i.e., feedback-inhibition) in a particular cell. In this context, a “feedback-deregulated” enzyme is a form of the enzyme that is less sensitive to feedback-inhibition than the enzyme native to the cell or a form of the enzyme that is native to the cell but is naturally less sensitive to feedback inhibition than one or more other natural forms of the enzyme. A feedback-deregulated enzyme may be produced by introducing one or more mutations into a native enzyme. Alternatively, a feedback-deregulated enzyme may simply be a heterologous, native enzyme that, when introduced into a particular microbial cell, is not as sensitive to feedback-inhibition as the native, native enzyme. In some embodiments, the feedback-deregulated enzyme shows no feedback-inhibition in the microbial cell.

The term “cystathionine” refers to a chemical compound of the formula C₇H₁₄N₂O₄S also known as “S-((R)-2-amino-2-carboxyethyl)-L-homocysteine” and “L-cystathionine” (CAS# CAS 56-88-2).

The term “sequence identity,” in the context of two or more amino acid or nucleotide sequences, refers to two or more sequences that are the same or have a specified percentage of amino acid residues or nucleotides that are the same, when compared and aligned for maximum correspondence, as measured using a sequence comparison algorithm or by visual inspection.

For sequence comparison to determine percent nucleotide or amino acid sequence identity, typically one sequence acts as a “reference sequence,” to which a “test” sequence is compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence relative to the reference sequence, based on the designated program parameters. Alignment of sequences for comparison can be conducted using BLAST set to default parameters.

The term “titer,” as used herein, refers to the mass of a product (e.g., cystathionine) produced by a culture of microbial cells divided by the culture volume.

As used herein with respect to recovering cystathionine from a cell culture, “recovering” refers to separating the cystathionine from at least one other component of the cell culture medium.

### Engineering Microbes for Cystathionine Production

Cystathionine Biosynthesis Pathway

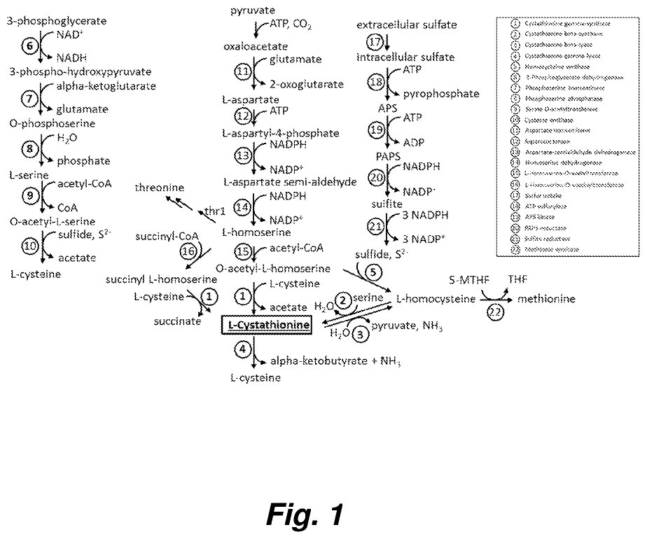

L-cystathionine can be derived from L-homocysteine in one enzymatic step, carried out by the enzyme cystathionine beta-synthase (enzyme 2 in FIG. 1). Alternatively, L-cystathionine can be derived from L-acetyl-L-homoserine or succinyl L-homoserine in one enzymatic step, carried out by the enzyme cystathionine gamma-synthase (enzyme 1 in FIG. 1). Cystathionine production can be enhanced in microbial hosts having one or both of these enzymes by introducing at least one of these enzymes, heterologously, into the host cell.

Engineering for Microbial Cystathionine Production

Any cystathionine beta-synthase or cystathionine gamma-synthase (referred to collectively as a “cystathionine synthase,” for ease of discussion) that is active in the microbial cell being engineered may be introduced into the cell, typically by introducing and expressing the gene(s) encoding the enzyme(s)s using standard genetic engineering techniques. Suitable cystathionine synthases may be derived from any source, including plant, archaeal, fungal, gram-positive bacterial, and gram-negative bacterial sources. Exemplary sources include, but are not limited to: Escherichia coli, Vibrio cholerae, Candidatus Burkholderia crenata, butyrate-producing bacterium, a Clostridium species (e.g., Clostridium CAG:221, Clostridium CAG:288), Staphylococcus aureus, Yersinia enterocolitica, Castellaniella detragans, and Prochorococcus marinus.

One or more copies of any of these genes can be introduced into a selected microbial host cell. If more than one copy of a gene is introduced, the copies can have the same or different nucleotide sequences. In some embodiments, one or both (or all) of the heterologous gene(s) is/are expressed from a strong, constitutive promoter. In some embodiments, the heterologous gene(s) is/are expressed from an inducible promoter. The heterologous gene(s) can optionally be codon-optimized to enhance expression in the selected microbial host cell. The codon-optimization tables used in the Examples are as follows: Bacillus subtilis Kazusa codon table:

Yarrowia lipolytica Kazusa codon table: www.kazusa.or.jp/codon/cgi-bin/showcodon.cgi?species=1423&aa=1&style=N, Yarrowia lipolytica Kazusa codon table: www.kazusa.or.jp/codon/cgi-bin/showcodon.cgi?species=4592&aa=1&style=N; Corynebacteria glutamicum Kazusa codon table: www.kazusa.or.jp/codon/cgi-bin/showcodon.cgi?species=340322&aa=1&style=N; Saccharomyces cerevisiae Kazusa codon table: www.kazusa.or.jp/codon/cgi-bin/showcodon.cgi!species=4932&aa=1&style=N. Also used, was a modified, combined codon usage scheme for S. cereviae and C. glutamicum, which is reproduced below.

| Amino Acid | Codon | Fraction |
|---|---|---|
| A | GCG | 0.22 |
| A | GCA | 0.29 |
| A | GCT | 0.24 |
| A | GCC | 0.25 |
| C | TGT | 0.36 |
| C | TGC | 0.64 |
| D | GAT | 0.56 |
| D | GAC | 0.44 |
| E | GAG | 0.44 |
| E | GAA | 0.56 |
| F | TTT | 0.37 |
| F | TTC | 0.63 |
| G | GGG | 0.08 |
| G | GGA | 0.19 |
| G | GGT | 0.3 |
| G | GGC | 0.43 |
| H | CAT | 0.32 |
| H | CAC | 0.68 |
| I | ATA | 0.03 |
| I | ATT | 0.38 |
| I | ATC | 0.59 |
| K | AAG | 0.6 |
| K | AAA | 0.4 |
| L | TTG | 0.29 |
| L | TTA | 0.05 |
| L | CTG | 0.29 |
| L | CTA | 0.06 |
| L | CTT | 0.17 |
| L | CTC | 0.14 |
| M | ATG | 1 |
| N | AAT | 0.33 |
| N | AAC | 0.67 |
| P | CCG | 0.22 |
| P | CCA | 0.35 |
| P | CCT | 0.23 |
| P | CCC | 0.2 |
| Q | CAG | 0.61 |
| Q | CAA | 0.39 |
| R | AGG | 0.11 |
| R | AGA | 0.12 |
| R | CGG | 0.09 |
| R | CGA | 0.17 |
| R | CGT | 0.34 |
| R | CGC | 0.18 |
| S | AGT | 0.08 |
| S | AGC | 0.16 |
| S | TCG | 0.12 |
| S | TCA | 0.13 |
| S | TCT | 0.17 |
| S | TCC | 0.34 |
| T | ACG | 0.14 |
| T | ACA | 0.12 |
| T | ACT | 0.2 |
| T | ACC | 0.53 |
| V | GTG | 0.36 |
| V | GTA | 0.1 |
| V | GTT | 0.26 |
| V | GTC | 0.28 |
| W | TGG | 1 |
| Y | TAT | 0.34 |
| Y | TAC | 0.66 |

In Saccharomyces cerevisiae, for example, an about 48 μg/L titer of cystathionine was achieved in a first round of engineering to express an S. cerevisiae cystathionine beta-synthase (UniProt ID N1P5Z1) using a constitutive promoter.

Increasing the Activity of Upstream Enzymes

One approach to increasing cystathionine production in a microbial cell that is capable of such production is to increase the activity of one or more upstream enzymes in the cystathionine biosynthesis pathway. Upstream pathway enzymes include all enzymes involved in the conversions from a feedstock all the way to a metabolite that can be directly converted to cystathionine (e.g., homocysteine, L-acetyl-L-homoserine, or succinyl L-homoserine). Illustrative enzymes, for this purpose, include, but are not limited to, those shown in FIG. 1 in the pathways leading to these metabolites. Suitable upstream pathway genes encoding these enzymes may be derived from any available source, including, for example, those discussed above as sources for a cystathionine synthase and disclosed elsewhere herein.

In some embodiments, the activity of one or more upstream pathway enzymes is increased by modulating the expression or activity of the native enzyme(s). For example, native regulators of the expression or activity of such enzymes can be exploited to increase the activity of suitable enzymes.

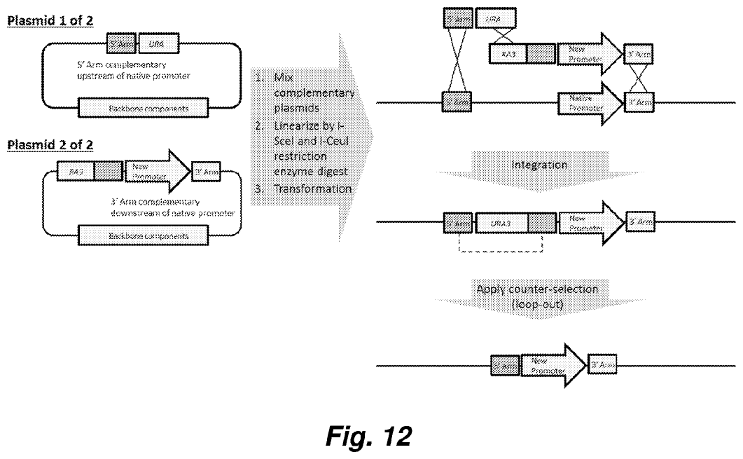

Alternatively, or in addition, one or more promoters can be substituted for native promoters using, for example, a technique such as that illustrated in FIG. 12. In certain embodiments, the replacement promoter is stronger than the native promoter and/or is a constitutive promoter.

In some embodiments, the activity of one or more upstream pathway enzymes is supplemented by introducing one or more of the corresponding genes into the engineered microbial host cell. An introduced upstream pathway gene may be from an organism other than that of the host cell or may simply be an additional copy of a native gene. In some embodiments, one or more such genes are introduced into a microbial host cell capable of cystathionine production and expressed from a strong constitutive promoter and/or can optionally be codon-optimized to enhance expression in the selected microbial host cell.

In various embodiments, the engineering of a cystathionine-producing microbial cell to increase the activity of one or more upstream pathway enzymes increases the cystathionine titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in cystathionine titer is in the range of 10-fold to 1000-fold, 20-fold to 500-fold, 50-fold to 400-fold, 10-fold to 300-fold, or any range bounded by any of the values listed above. (Ranges herein include their endpoints.) These increases are determined relative to the cystathionine titer observed in a cystathionine-producing microbial cell that lacks any increase in activity of upstream pathway enzymes. This reference cell may have one or more other genetic alterations aimed at increasing cystathionine production.

In various embodiments, the cystathionine titers achieved by increasing the activity of one or more upstream pathway enzymes are at least 10, 20, 30, 40, 50, 75, 100, 200, 300, 400, 500, 600, 700, 800, or 900 μg/L or at least 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 110, 120, 130, 140, or 150 mg/L. In various embodiments, the titer is in the range of 50 μg/L to 100 mg/L, 75 μg/L to 75 mg/L, 100 μg/L to 50 mg/L, 200 μg/L to 25 mg/L, 300 μg/L to 10 mg/L, 350 μg/L to 5 mg/L or any range bounded by any of the values listed above.

Feedback-Deregulated Enzymes

Another approach to increasing cystathionine production in a microbial cell engineered for enhanced cystathionine production is to introduce feedback-deregulated forms of one or more enzymes that are normally subject to feedback regulation. A feedback-deregulated form can be a heterologous, native enzyme that is less sensitive to feedback inhibition than the native enzyme in the particular microbial host cell. Alternatively, a feedback-deregulated form can be a variant of a native or heterologous enzyme that has one or more mutations or truncations rendering it less sensitive to feedback inhibition than the corresponding native enzyme.

In some embodiments, the feedback-deregulated enzyme need not be “introduced,” in the traditional sense. Rather, the microbial host cell selected for engineering can be one that has a native enzyme that is naturally insensitive to feedback inhibition.

In various embodiments, the engineering of a cystathionine-producing microbial cell to include one or more feedback-regulated enzymes increases the cystathionine titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in cystathionine titer is in the range of 10-fold to 1000-fold, 20-fold to 500-fold, 50-fold to 400-fold, 10-fold to 300-fold, or any range bounded by any of the values listed above. These increases are determined relative to the cystathionine titer observed in a cystathionine-producing microbial cell that does not include genetic alterations to reduce feedback regulation. This reference cell may (but need not) have other genetic alterations aimed at increasing cystathionine production, i.e., the cell may have increased activity of an upstream pathway enzyme.

In various embodiments, the cystathionine titers achieved by reducing feedback deregulation are at least 10, 20, 30, 40, 50, 75, 100, 200, 300, 400, 500, 600, 700, 800, or 900 μg/L or at least 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 110, 120, 130, 140, or 150 mg/L. In various embodiments, the titer is in the range of 50 μg/L to 100 mg/L, 75 μg/L to 75 mg/L, 100 μg/L to 50 mg/L, 200 μg/L to 25 mg/L, 300 μg/L to 10 mg/L, 350 μg/L to 5 mg/L or any range bounded by any of the values listed above.

Reduction of Consumption of Cystathionine and/or Its Precursors

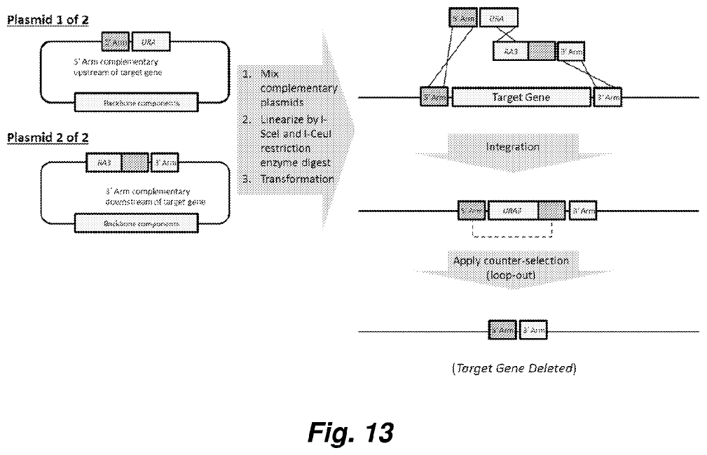

Another approach to increasing cystathionine production in a microbial cell that is capable of such production is to decrease the activity of one or more enzymes that consume one or more cystathionine pathway precursors or that consume cystathionine itself. In some embodiments, the activity of one or more such enzymes is reduced by modulating the expression or activity of the native enzyme(s). Illustrative enzymes of this type include homoserine dehydrogenase and cell wall biosynthesis pathway genes. The activity of such enzymes can be decreased, for example, by substituting the native promoter of the corresponding gene(s) with a less active or inactive promoter or by deleting the corresponding gene(s). See FIGS. 12 and 13 for examples of schemes for promoter replacement and targeted gene deletion, respectively, in S. cervisiae and Y. lipolytica.

In various embodiments, the engineering of a cystathionine-producing microbial cell to reduce precursor consumption by one or more side pathways increases the cystathionine titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in cystathionine titer is in the range of 10-fold to 1000-fold, 20-fold to 500-fold, 50-fold to 400-fold, 10-fold to 300-fold, or any range bounded by any of the values listed above. These increases are determined relative to the cystathionine titer observed in a cystathionine-producing microbial cell that does not include genetic alterations to reduce precursor consumption. This reference cell may (but need not) have other genetic alterations aimed at increasing cystathionine production, i.e., the cell may have increased activity of an upstream pathway enzyme.

In various embodiments, the cystathionine titers achieved by reducing precursor consumption are at least 10, 20, 30, 40, 50, 75, 100, 200, 300, 400, 500, 600, 700, 800, or 900 μg/L or at least 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 110, 120, 130, 140, or 150 mg/L. In various embodiments, the titer is in the range of 50 μg/L to 100 mg/L, 75 μg/L to 75 mg/L, 100 μg/L to 50 mg/L, 200 μg/L to 25 mg/L, 300 μg/L to 10 mg/L, 350 μg/L to 5 mg/L or any range bounded by any of the values listed above.

Any of the approaches for increasing cystathionine production described above can be combined, in any combination, to achieve even higher cystathionine production levels.

Expression of a Cystathionine Transporter

In some embodiments, it is advantageous to recover cystathionine from culture medium. To enhance transport of this compound from inside the engineered microbial cell to the culture medium, an amino acid transporter that can export cystathionine and is active in the microbial cell being engineered may be introduced into the cell, typically by introducing and expressing the gene(s) encoding the enzyme(s)s using standard genetic engineering techniques. Suitable cystathionine transporters may be derived from any available source including for example, Escherichia coli.

Altering the Cofactor Specificity of Upstream Pathway Enzymes

Another approach to increasing cystathionine production in a microbial cell that is capable of such production is to alter the cofactor specificity of an upstream pathway enzyme that typically prefers the reduced form of nicotinamide adenine dinucleotide phosphate (NADPH) to the reduced from of nicotinamide adenine dinucleotide (NADH). which provides the reducing equivalents for biosynthetic reactions. This can be achieved, for example, by expressing one or more variants of such enzymes that have the desired altered cofactor specificity. Examples of upstream pathway enzymes that rely on NADPH, and for which suitable variants are known, include aspartate semi-aldehyde dehydrogenase, homoserine dehydrogenase, and glyceraldehyde 3-phosphate dehydrogenase (GAPDH).

In various embodiments, the engineering of a cystathionine-producing microbial cell to alter the cofactor specificity of one or more of such enzymes increases the cystathionine titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in cystathionine titer is in the range of 10-fold to 1000-fold, 20-fold to 500-fold, 50-fold to 400-fold, 10-fold to 300-fold, or any range bounded by any of the values listed above. (Ranges herein include their endpoints.) These increases are determined relative to the cystathionine titer observed in a cystathionine-producing microbial cell that lacks any increase in activity of such enzymes. This reference cell may have one or more other genetic alterations aimed at increasing cystathionine production.

In various embodiments, the cystathionine titers achieved by altering the cofactor specificity of one or more enzymes that typically rely on NADPH as a cofactor are at least 10, 20, 30, 40, 50, 75, 100, 200, 300, 400, 500, 600, 700, 800, or 900 μg/L or at least 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 110, 120, 130, 140, or 150 mg/L. In various embodiments, the titer is in the range of 50 μg/L to 100 mg/L, 75 μg/L to 75 mg/L, 100 μg/L to 50 mg/L, 200 μg/L to 25 mg/L, 300 μg/L to 10 mg/L, 350 μg/L to 5 mg/L or any range bounded by any of the values listed above.

Illustrative Amino Acid and Nucleotide Sequences

The following table identifies amino acid and nucleotide sequences used in Example 1. The corresponding sequences are shown in the Sequence Listing.

SEQ ID NO Cross-Reference Table

| | AA SEQ |
|---|---|
| Enzyme Description | ID NO: |
| Cystathionine beta-synthase enzyme from | 1 |
| Saccharomyces cerevisiae (strain CEN.PK113-7D) | |
| (UniProt ID N1P5Z1) | |
| Cystathionine gamma-synthase enzyme from | 2 |
| Escherichia coli (UniProt ID P00935) | |
| Aspartate aminotransferase enzyme from | 3 |
| Saccharomyces cerevisiae (strain CEN.PK113-7D) | |
| (UniProt ID N1NZ14) | |
| Feedback-Deregulated (G452D) | 4 |
| Aspartate kinase from | |
| Saccharomyces cerevisiae (UniProt ID P10869) | |
| Feedback-Deregulated (G378E) | 5 |
| Homoserine dehydrogenase from | |
| Corynebacterium glutamicum | |
| Cystathionine gamma-synthase/ | 6 |
| O-acetylhomoserine enzyme from | |
| Bacillus subtilis | |
| Feedback-Deregulated (A279T) | 7 |
| Aspartokinase from | |
| Corynebacterium glutamicum | |
| Feedback-Deregulated (G378S) | 8 |
| Homoserine dehydrogenase from | |
| Corynebacterium glutamicum | |
| Feedback-Deregulated (S345F) | 9 |
| Bifunctional aspartokinase/ | |
| homoserine dehydrogenase from Escherichia coli | |
| Putative O-acetylhomoserine | 10 |
| aminocarboxypropyltransferase from | |
| Corynebacterium glutamicum | |
| Cystathionine gamma-synthase from | 11 |
| Bacillus paralicheniformis ATCC 9945a | |
| (UniProt ID R9TW27) | |

Microbial Host Cells

Any microbe that can be used to express introduced genes can be engineered for fermentative production of cystathionine as described above. In certain embodiments, the microbe is one that is naturally incapable of fermentative production of cystathionine. In some embodiments, the microbe is one that is readily cultured, such as, for example, a microbe known to be useful as a host cell in fermentative production of compounds of interest. Bacteria cells, including gram-positive or gram-negative bacteria can be engineered as described above. Examples include, in addition to C. glutamicum cells, Bacillus subtilus, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, S. albus, S. lividans, S. coelicolor, S. griseus, Pseudomonas sp., P. alcaligenes, P. citrea, Lactobacilis spp. (such as L. lactis, L. plantarum), L. grayi, E. coli, E. faecium, E. gallinarum, E. casseliflavus, and/or E. faecalis cells.

There are numerous types of anaerobic cells that can be used as microbial host cells in the methods described herein. In some embodiments, the microbial cells are obligate anaerobic cells. Obligate anaerobes typically do not grow well, if at all, in conditions where oxygen is present. It is to be understood that a small amount of oxygen may be present, that is, there is some level of tolerance level that obligate anaerobes have for a low level of oxygen. Obligate anaerobes engineered as described above can be grown under substantially oxygen-free conditions, wherein the amount of oxygen present is not harmful to the growth, maintenance, and/or fermentation of the anaerobes.

Alternatively, the microbial host cells used in the methods described herein can be facultative anaerobic cells. Facultative anaerobes can generate cellular ATP by aerobic respiration (e.g., utilization of the TCA cycle) if oxygen is present. However, facultative anaerobes can also grow in the absence of oxygen. Facultative anaerobes engineered as described above can be grown under substantially oxygen-free conditions, wherein the amount of oxygen present is not harmful to the growth, maintenance, and/or fermentation of the anaerobes, or can be alternatively grown in the presence of greater amounts of oxygen.

In some embodiments, the microbial host cells used in the methods described herein are filamentous fungal cells. (See, e.g., Berka & Barnett, Biotechnology Advances, (1989), 7(2):127-154). Examples include Trichoderma longibrachiatum, T viride, T koningii, T. harzianum, Penicillium sp., Humicola insolens, H. lanuginose, H. grisea, Chrysosporium sp., C. lucknowense, Gliocladium sp., Aspergillus sp. (such as A. oryzae, A. niger, A. sojae, A. japonicus, A. nidulans, or A. awamori), Fusarium sp. (such as F. roseum, F. graminum F. cerealis, F. oxysporuim, or F. venenatum), Neurospora sp. (such as N. crassa or Hypocrea sp.), Mucor sp. (such as M. miehei), Rhizopus sp., and Emericella sp. cells. In particular embodiments, the fungal cell engineered as described above is A. nidulans, A. awamori, A. oryzae, A. aculeatus, A. niger, A. japonicus, T reesei, T. viride, F. oxysporum, or F. solani. Illustrative plasmids or plasmid components for use with such hosts include those described in U.S. Patent Pub. No. 2011/0045563.

Yeasts can also be used as the microbial host cell in the methods described herein. Examples include: Saccharomyces sp., Schizosaccharomyces sp., Pichia sp., Hansenula polymorpha, Pichia stipites, Kluyveromyces marxianus, Kluyveromyces spp., Yarrowia lipolytica and Candida sp. In some embodiments, the Saccharomyces sp. is S. cerevisiae (See, e.g., Romanos et al., Yeast, (1992), 8(6):423-488). Illustrative plasmids or plasmid components for use with such hosts include those described in U.S. Pat. No. 7,659,097 and U.S. Patent Pub. No. 2011/0045563.

In some embodiments, the host cell can be an algal cell derived, e.g., from a green alga, red alga, a glaucophyte, a chlorarachniophyte, a euglenid, a chromista, or a dinoflagellate. (See, e.g., Saunders & Warmbrodt, “Gene Expression in Algae and Fungi, Including Yeast,” (1993), National Agricultural Library, Beltsville, Md.). Illustrative plasmids or plasmid components for use in algal cells include those described in U.S. Patent Pub. No. 2011/0045563.

In other embodiments, the host cell is a cyanobacterium, such as cyanobacterium classified into any of the following groups based on morphology: Chlorococcales, Pleurocapsales, Oscillatoriales, Nostocales, Synechosystic or Stigonematales (See, e.g., Lindberg et al., Metab. Eng., (2010) 12(1):70-79). Illustrative plasmids or plasmid components for use in cyanobacterial cells include those described in U.S. Patent Pub. Nos. 2010/0297749 and 2009/0282545 and in Intl. Pat. Pub. No. WO 2011/034863.

Genetic Engineering Methods

Microbial cells can be engineered for fermentative cystathionine production using conventional techniques of molecular biology (including recombinant techniques), microbiology, cell biology, and biochemistry, which are within the skill of the art. Such techniques are explained fully in the literature, see e.g., “Molecular Cloning: A Laboratory Manual,” fourth edition (Sambrook et al., 2012); “Oligonucleotide Synthesis” (M. J. Gait, ed., 1984); “Culture of Animal Cells: A Manual of Basic Technique and Specialized Applications” (R. I. Freshney, ed., 6th Edition, 2010); “Methods in Enzymology” (Academic Press, Inc.); “Current Protocols in Molecular Biology” (F. M. Ausubel et al., eds., 1987, and periodic updates); “PCR: The Polymerase Chain Reaction,” (Mullis et al., eds., 1994); Singleton et al., Dictionary of Microbiology and Molecular Biology 2nd ed., J. Wiley & Sons (New York, N.Y. 1994).

Vectors are polynucleotide vehicles used to introduce genetic material into a cell. Vectors useful in the methods described herein can be linear or circular. Vectors can integrate into a target genome of a host cell or replicate independently in a host cell. For many applications, integrating vectors that produced stable transformants are preferred. Vectors can include, for example, an origin of replication, a multiple cloning site (MCS), and/or a selectable marker. An expression vector typically includes an expression cassette containing regulatory elements that facilitate expression of a polynucleotide sequence (often a coding sequence) in a particular host cell. Vectors include, but are not limited to, integrating vectors, prokaryotic plasmids, episomes, viral vectors, cosmids, and artificial chromosomes.

Illustrative regulatory elements that may be used in expression cassettes include promoters, enhancers, internal ribosomal entry sites (IRES), and other expression control elements (e.g., transcription termination signals, such as polyadenylation signals and poly-U sequences). Such regulatory elements are described, for example, in Goeddel, Gene Expression Technology: Methods In Enzymology 185, Academic Press, San Diego, Calif. (1990).

In some embodiments, vectors may be used to introduce systems that can carry out genome editing, such as CRISPR systems. See U.S. Patent Pub. No. 2014/0068797, published 6 Mar. 2014; see also Jinek M., et al., “A programmable dual-RNA-guided DNA endonuclease in adaptive bacterial immunity,” Science 337:816-21, 2012). In Type II CRISPR-Cas9 systems, Cas9 is a site-directed endonuclease, namely an enzyme that is, or can be, directed to cleave a polynucleotide at a particular target sequence using two distinct endonuclease domains (HNH and RuvC/RNase H-like domains). Cas9 can be engineered to cleave DNA at any desired site because Cas9 is directed to its cleavage site by RNA. Cas9 is therefore also described as an “RNA-guided nuclease.” More specifically, Cas9 becomes associated with one or more RNA molecules, which guide Cas9 to a specific polynucleotide target based on hybridization of at least a portion of the RNA molecule(s) to a specific sequence in the target polynucleotide. Ran, F. A., et al., (“In vivo genome editing using Staphylococcus aureus Cas9,” Nature 520(7546):186-91, 2015, Apr. 9], including all extended data) present the crRNA/tracrRNA sequences and secondary structures of eight Type II CRISPR-Cas9 systems. Cas9-like synthetic proteins are also known in the art (see U.S. Published Patent Application No. 2014-0315985, published 23 Oct. 2014).

Example 1 describes illustrative integration approaches for introducing polynucleotides and other genetic alterations into the genomes of C. glutamicum, S. cerevisiae, and B. subtilis cells.

Vectors or other polynucleotides can be introduced into microbial cells by any of a variety of standard methods, such as transformation, conjugation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion. Transformants can be selected by any method known in the art. Suitable methods for selecting transformants are described in U.S. Patent Pub. Nos. 2009/0203102, 2010/0048964, and 2010/0003716, and International Publication Nos. WO 2009/076676, WO 2010/003007, and WO 2009/132220.

### Engineered Microbial Cells

The above-described methods can be used to produce engineered microbial cells that produce, and in certain embodiments, overproduce, cystathionine. Engineered microbial cells can have at least 1, 2, 3, 4, 5, 6, 7, 8, 9, 10, 20, 30, 40, 50, 60, 70, 80, 90, 100 or more genetic alterations, such as 30-100 alterations, as compared to a native microbial cell, such as any of the microbial host cells described herein. Engineered microbial cells described in the Example below have one, two, or three genetic alterations, but those of skill in the art can, following the guidance set forth herein, design microbial cells with additional alterations. In some embodiments, the engineered microbial cells have not more than 15, 14, 13, 12, 11, 10, 9, 8, 7, 6, 5, or 4 genetic alterations, as compared to a native microbial cell. In various embodiments, microbial cells engineered for cystathionine production can have a number of genetic alterations falling within the any of the following illustrative ranges: 1-10, 1-9, 1-8, 2-7, 2-6, 2-5, 2-4, 2-3, 3-7, 3-6, 3-5, 3-4, etc.

In some embodiments, an engineered microbial cell expresses at least one heterologous cystathionine synthase. In various embodiments, the microbial cell can include and express, for example: (1) a single heterologous cystathionine synthase gene, (2) two or more heterologous cystathionine synthase genes, which can be the same or different (in other words, multiple copies of the same heterologous cystathionine synthase gene can be introduced or multiple, different heterologous cystathionine synthase genes can be introduced), (3) a single heterologous cystathionine synthase gene that is not native to the cell and one or more additional copies of an native cystathionine synthase gene (if applicable), or (4) two or more non-native cystathionine synthase genes, which can be the same or different, and one or more additional copies of a native cystathionine beta-synthase gene (if applicable).

This engineered host cell can include at least one additional genetic alteration that increases flux through any pathway leading to the production of an immediate precursor of cystathionine. As discussed above, this can be accomplished by one or more of the following: increasing the activity of upstream enzymes, reducing consumption of cystathionine precursors or a cystathionine itself, and altering the cofactor specificity of upstream pathway enzymes.

In addition, the engineered host cell can express an amino acid transporter to enhance transport of cystathionine from inside the engineered microbial cell to the culture medium.

The engineered microbial cells can contain introduced genes that have a native nucleotide sequence or that differ from native. For example, the native nucleotide sequence can be codon-optimized for expression in a particular host cell. Codon optimization for a particular host can, for example, be based on the codon usage tables found at www.kazusa.or.jp/codon/. The amino acid sequences encoded by any of these introduced genes can be native or can differ from native. In various embodiments, the amino acid sequences have at least 60 percent, 70 percent, 75 percent, 80 percent, 85 percent, 90 percent, 95 percent or 100 percent amino acid sequence identity with a native amino acid sequence.

The approach described herein has been carried out in bacterial cells, namely C. glutamicum and B. subtilis (prokaryotes), and in fungal cells, namely the yeasts S. cerevisiae and Y. lypolytica (eukaryotes). (See Examples 1 and 2.)

Illustrative Engineered Bacterial Cells

In certain embodiments, the engineered bacterial (e.g., C. glutamicum) cell expresses one or more heterologous cystathionine beta-synthase(s) having at least 70 percent, 75 percent, 80 percent, 85 percent, 90 percent, 95 percent or 100 percent amino acid sequence identity with a cystathionine beta-synthase from S. cerevisiae (UniProt ID N1P5Z1); and/or one or more heterologous cystathionine gamma-synthase(s) having at least 70 percent, 75 percent, 80 percent, 85 percent, 90 percent, 95 percent or 100 percent amino acid sequence identity with a cystathionine gamma-synthase from E. coli K12 (UniProt ID P00935); and/or or one or more heterologous aspartate aminotransferase(s) having at least 70 percent, 75 percent, 80 percent, 85 percent, 90 percent, 95 percent or 100 percent amino acid sequence identity with an aspartate aminotransferase from S. cerevisiae CEN.PK113-7D (UniProt ID N1NZ14).

In particular embodiments:
the cystathionine beta-synthase from S. cerevisiae (UniProt ID N1P5Z1) SEQ ID NO:1;
the cystathionine gamma-synthase from E. coli K12 (UniProt ID P00935) includes SEQ ID NO:2; and/or
the aspartate aminotransferase from S. cerevisiae CEN.PK113-7D (UniProt ID N1NZ14) includes SEQ ID NO:3.

In an illustrative embodiment, a titer of about 4.0 mg/L was achieved after engineering C. glutamicum, to express cystathionine beta-synthase from S. cerevisiae (UniProt ID N1P5Z1), cystathionine gamma-synthase from E. coli K12 (UniProt ID P00935), and aspartate aminotransferase from S. cerevisiae CEN.PK113-7D (UniProt ID N1NZ14).

In certain embodiments, the engineered bacterial (e.g., B. subtilis) cell expresses one or more heterologous cystathionine beta-synthase(s) having at least 70 percent, 75 percent, 80 percent, 85 percent, 90 percent, 95 percent or 100 percent amino acid sequence identity with a cystathionine beta-synthase from S. cerevisiae (UniProt ID N1P5Z1); and/or one or more heterologous cystathionine gamma-synthase(s) having at least 70 percent, 75 percent, 80 percent, 85 percent, 90 percent, 95 percent or 100 percent amino acid sequence identity with a cystathionine gamma-synthase from B. paralicheniformis ATCC 9945a (UniProt ID R9TW27); and/or one or more feedback-deregulated aspartokinase(s) having at least 70 percent, 75 percent, 80 percent, 85 percent, 90 percent, 95 percent or 100 percent amino acid sequence identity with a feedback-deregulated aspartokinase from S. cerevisiae S288c (UniProt ID P10869), harboring the amino acid substitution G452D.

In particular embodiments:
the cystathionine beta-synthase from S. cerevisiae (UniProt ID N1P5Z1) includes SEQ ID NO:1;
the cystathionine gamma-synthase from B. paralicheniformis ATCC 9945a (UniProt ID R9TW27) includes SEQ ID NO:11; and/or
the feedback-deregulated aspartokinase from S. cerevisiae S288c (UniProt ID P10869), harboring the amino acid substitution G452D, includes SEQ ID NO:4.

In an illustrative embodiment, a titer of about 1.0 mg/L was achieved after engineering B. subtilis to express cystathionine beta-synthase from S. cerevisiae (UniProt ID N1P5Z1), cystathionine gamma-synthase from B. paralicheniformis ATCC 9945a (UniProt ID R9TW27), and feedback-deregulated aspartokinase from S. cerevisiae S288c (UniProt ID P10869), harboring the amino acid substitution G452D.

Illustrative Engineered Yeast Cells

In certain embodiments, the engineered yeast (e.g., S. cerevisiae) cell expresses one or more heterologous cystathionine beta-synthase(s) having at least 70 percent, 75 percent, 80 percent, 85 percent, 90 percent, 95 percent or 100 percent amino acid sequence identity with a cystathionine beta-synthase from S. cerevisiae (UniProt ID N1P5Z1); and/or one or more heterologous cystathionine gamma-synthase(s) having at least 70 percent, 75 percent, 80 percent, 85 percent, 90 percent, 95 percent or 100 percent amino acid sequence identity with a cystathionine gamma-synthase from E. coli K12 (UniProt ID P00935); and/or or one or more one or more feedback-deregulated aspartokinase(s) having at least 70 percent, 75 percent, 80 percent, 85 percent, 90 percent, 95 percent or 100 percent amino acid sequence identity with a feedback-deregulated aspartokinase from S. cerevisiae S288c (UniProt ID P10869), harboring the amino acid substitution G452D.

In particular embodiments:
the cystathionine beta-synthase from S. cerevisiae (UniProt ID N1P5Z1) includes SEQ ID NO:1;
the cystathionine gamma-synthase from E. coli K12 (UniProt ID P00935 includes SEQ ID NO:2; and/or
the feedback-deregulated aspartokinase from S. cerevisiae S288c (UniProt ID P10869), harboring the amino acid substitution G452D, includes SEQ ID NO:4.

In an illustrative embodiment, a titer of about 360 μg/L was achieved after engineering S. cerevisiae to express cystathionine beta-synthase from S. cerevisiae (UniProt ID N1P5Z1), cystathionine gamma-synthase from Escherichia coli K12 (UniProt ID P00935), and feedback-deregulated aspartokinase from S. cerevisiae S288c (UniProt ID P10869), harboring the amino acid substitution G452D.

In certain embodiments, the engineered yeast (e.g., Y. lipolytica) cell expresses one or more heterologous cystathionine beta-synthase(s) having at least 70 percent, 75 percent, 80 percent, 85 percent, 90 percent, 95 percent or 100 percent amino acid sequence identity with a cystathionine beta-synthase from S. cerevisiae (UniProt ID N1P5Z1); and/or one or more heterologous cystathionine gamma-synthase(s) having at least 70 percent, 75 percent, 80 percent, 85 percent, 90 percent, 95 percent or 100 percent amino acid sequence identity with a cystathionine gamma-synthase from B. paralicheniformis ATCC 9945a (UniProt ID R9TW27); and/or one or more feedback-deregulated aspartokinase(s) having at least 70 percent, 75 percent, 80 percent, 85 percent, 90 percent, 95 percent or 100 percent amino acid sequence identity with a feedback-deregulated aspartokinase from S. cerevisiae S288c (UniProt ID P10869), harboring the amino acid substitution G452D.

In particular embodiments:
the cystathionine beta-synthase from S. cerevisiae (UniProt ID N1P5Z1) includes SEQ ID NO:1;
the cystathionine gamma-synthase from B. paralicheniformis ATCC 9945a (UniProt ID R9TW27) includes SEQ ID NO:11; and/or
the feedback-deregulated aspartokinase from S. cerevisiae S288c (UniProt ID P10869), harboring the amino acid substitution G452D, includes SEQ ID NO:4.

In an illustrative embodiment, a titer of about 92.5 μg/L was achieved after engineering Y. lipolytica to express cystathionine beta-synthase from S. cerevisiae (UniProt ID N1P5Z1), cystathionine gamma-synthase from Bacillus paralicheniformis ATCC 9945a (UniProt ID R9TW27), and feedback-deregulated aspartokinase from S. cerevisiae S288c (UniProt ID P10869), harboring the amino acid substitution G452D.

### Culturing of Engineered Microbial Cells

Any of the microbial cells described herein can be cultured, e.g., for maintenance, growth, and/or cystathionine production.

In some embodiments, the cultures are grown to an optical density at 600 nm of 10-500, such as an optical density of 50-150.

In various embodiments, the cultures include produced cystathionine at titers of at least 10, 20, 30, 40, 50, 75, 100, 200, 300, 400, 500, 600, 700, 800, or 900 μg/L or at least 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 110, 120, 130, 140, or 150 mg/L. In various embodiments, the titer is in the range of 50 μg/L to 100 mg/L, 75 μg/L to 75 mg/L, 100 μg/L to 50 gm/L, 200 μg/L to 25 gm/L, 300 μg/L to 10 gm/L, 350 μg/L to 5 gm/L or any range bounded by any of the values listed above.

Culture Media

Microbial cells can be cultured in any suitable medium including, but not limited to, a minimal medium, i.e., one containing the minimum nutrients possible for cell growth. Minimal medium typically contains: (1) a carbon source for microbial growth; (2) salts, which may depend on the particular microbial cell and growing conditions; and (3) water. Suitable media can also include any combination of the following: a nitrogen source for growth and product formation, a sulfur source for growth, a phosphate source for growth, metal salts for growth, vitamins for growth, and other cofactors for growth.

Any suitable carbon source can be used to cultivate the host cells. The term “carbon source” refers to one or more carbon-containing compounds capable of being metabolized by a microbial cell. In various embodiments, the carbon source is a carbohydrate (such as a monosaccharide, a disaccharide, an oligosaccharide, or a polysaccharide), or an invert sugar (e.g., enzymatically treated sucrose syrup). Illustrative monosaccharides include glucose (dextrose), fructose (levulose), and galactose; illustrative oligosaccharides include dextran or glucan, and illustrative polysaccharides include starch and cellulose. Suitable sugars include C6 sugars (e.g., fructose, mannose, galactose, or glucose) and C5 sugars (e.g., xylose or arabinose). Other, less expensive carbon sources include sugar cane juice, beet juice, sorghum juice, and the like, any of which may, but need not be, fully or partially deionized.

The salts in a culture medium generally provide essential elements, such as magnesium, nitrogen, phosphorus, and sulfur to allow the cells to synthesize proteins and nucleic acids.

Minimal medium can be supplemented with one or more selective agents, such as antibiotics.

To produce cystathionine, the culture medium can include, and/or is supplemented during culture with, glucose and/or a nitrogen source such as urea, an ammonium salt, ammonia, or any combination thereof.

Culture Conditions

Materials and methods suitable for the maintenance and growth of microbial cells are well known in the art. See, for example, U.S. Pub. Nos. 2009/0203102, 2010/0003716, and 2010/0048964, and International Pub. Nos. WO 2004/033646, WO 2009/076676, WO 2009/132220, and WO 2010/003007, Manual of Methods for General Bacteriology Gerhardt et al., eds), American Society for Microbiology, Washington, D.C. (1994) or Brock in Biotechnology: A Textbook of Industrial Microbiology, Second Edition (1989) Sinauer Associates, Inc., Sunderland, Mass.

In general, cells are grown and maintained at an appropriate temperature, gas mixture, and pH (such as about 20° C. to about 37° C., about 6% to about 84% CO₂, and a pH between about 5 to about 9). In some aspects, cells are grown at 35° C. In certain embodiments, such as where thermophilic bacteria are used as the host cells, higher temperatures (e.g., 50° C. -75° C.) may be used. In some aspects, the pH ranges for fermentation are between about pH 5.0 to about pH 9.0 (such as about pH 6.0 to about pH 8.0 or about 6.5 to about 7.0). Cells can be grown under aerobic, anoxic, or anaerobic conditions based on the requirements of the particular cell.

Standard culture conditions and modes of fermentation, such as batch, fed-batch, or continuous fermentation that can be used are described in U.S. Publ. Nos. 2009/0203102, 2010/0003716, and 2010/0048964, and International Pub. Nos. WO 2009/076676, WO 2009/132220, and WO 2010/003007. Batch and Fed-Batch fermentations are common and well known in the art, and examples can be found in Brock, Biotechnology: A Textbook of Industrial Microbiology, Second Edition (1989) Sinauer Associates, Inc.

In some embodiments, the cells are cultured under limited sugar (e.g., glucose) conditions. In various embodiments, the amount of sugar that is added is less than or about 105% (such as about 100%, 90%, 80%, 70%, 60%, 50%, 40%, 30%, 20%, or 10%) of the amount of sugar that can be consumed by the cells. In particular embodiments, the amount of sugar that is added to the culture medium is approximately the same as the amount of sugar that is consumed by the cells during a specific period of time. In some embodiments, the rate of cell growth is controlled by limiting the amount of added sugar such that the cells grow at the rate that can be supported by the amount of sugar in the cell medium. In some embodiments, sugar does not accumulate during the time the cells are cultured. In various embodiments, the cells are cultured under limited sugar conditions for times greater than or about 1, 2, 3, 5, 10, 15, 20, 25, 30, 35, 40, 50, 60, or 70 hours or even up to about 5-10 days. In various embodiments, the cells are cultured under limited sugar conditions for greater than or about 5, 10, 15, 20, 25, 30, 35, 40, 50, 60, 70, 80, 90, 95, or 100% of the total length of time the cells are cultured. While not intending to be bound by any particular theory, it is believed that limited sugar conditions can allow more favorable regulation of the cells.

In some aspects, the cells are grown in batch culture. The cells can also be grown in fed-batch culture or in continuous culture. Additionally, the cells can be cultured in minimal medium, including, but not limited to, any of the minimal media described above. The minimal medium can be further supplemented with 1.0% (w/v) glucose (or any other six-carbon sugar) or less. Specifically, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose. In some cultures, significantly higher levels of sugar (e.g., glucose) are used, e.g., at least 10% (w/v), 20% (w/v), 30% (w/v), 40% (w/v), 50% (w/v), 60% (w/v), 70% (w/v), or up to the solubility limit for the sugar in the medium. In some embodiments, the sugar levels falls within a range of any two of the above values, e.g.: 0.1-10% (w/v), 1.0-20% (w/v), 10-70% (w/v), 20-60% (w/v), or 30-50% (w/v). Furthermore, different sugar levels can be used for different phases of culturing. For fed-batch culture (e.g., of S. cerevisiae or C. glutamicum), the sugar level can be about 100-200 g/L (10-20% (w/v)) in the batch phase and then up to about 500-700 g/L (50-70% in the feed).

Additionally, the minimal medium can be supplemented 0.1% (w/v) or less yeast extract. Specifically, the minimal medium can be supplemented with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. Alternatively, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose and with 0.1% (w/v), 0.09% (w/v), 0.08% (w/v), 0.07% (w/v), 0.06% (w/v), 0.05% (w/v), 0.04% (w/v), 0.03% (w/v), or 0.02% (w/v) yeast extract. In some cultures, significantly higher levels of yeast extract can be used, e.g., at least 1.5% (w/v), 2.0% (w/v), 2.5% (w/v), or 3% (w/v). In some cultures (e.g., of S. cerevisiae or C. glutamicum), the yeast extract level falls within a range of any two of the above values, e.g.: 0.5-3.0% (w/v), 1.0-2.5% (w/v), or 1.5-2.0% (w/v).

Illustrative materials and methods suitable for the maintenance and growth of the engineered microbial cells described herein can be found below in Example 1.

### Cystathionine Production and Recovery

Any of the methods described herein may further include a step of recovering cystathionine. In some embodiments, the produced cystathionine contained in a so-called harvest stream is recovered/harvested from the production vessel. The harvest stream may include, for instance, cell-free or cell-containing aqueous solution coming from the production vessel, which contains cystathionine as a result of the conversion of production substrate by the resting cells in the production vessel. Cells still present in the harvest stream may be separated from the cystathionine by any operations known in the art, such as for instance filtration, centrifugation, decantation, membrane crossflow ultrafiltration or microfiltration, tangential flow ultrafiltration or microfiltration or dead-end filtration. After this cell separation operation, the harvest stream is essentially free of cells.

Further steps of separation and/or purification of the produced cystathionine from other components contained in the harvest stream, i.e., so-called downstream processing steps may optionally be carried out. These steps may include any means known to a skilled person, such as, for instance, concentration, extraction, crystallization, precipitation, adsorption, ion exchange, and/or chromatography. Any of these procedures can be used alone or in combination to purify cystathionine. Further purification steps can include one or more of, e.g., concentration, crystallization, precipitation, washing and drying, treatment with activated carbon, ion exchange, nanofiltration, and/or re-crystallization. The design of a suitable purification protocol may depend on the cells, the culture medium, the size of the culture, the production vessel, etc. and is within the level of skill in the art.

The following examples are given for the purpose of illustrating various embodiments of the disclosure and are not meant to limit the present disclosure in any fashion. Changes therein and other uses which are encompassed within the spirit of the disclosure, as defined by the scope of the claims, will be identifiable to those skilled in the art.

### EXAMPLE 1

### Construction and Selection of Strains of Corynebacteria glutamicum, and Saccharomyces cerevisiae Engineered to Produce Cystathionine

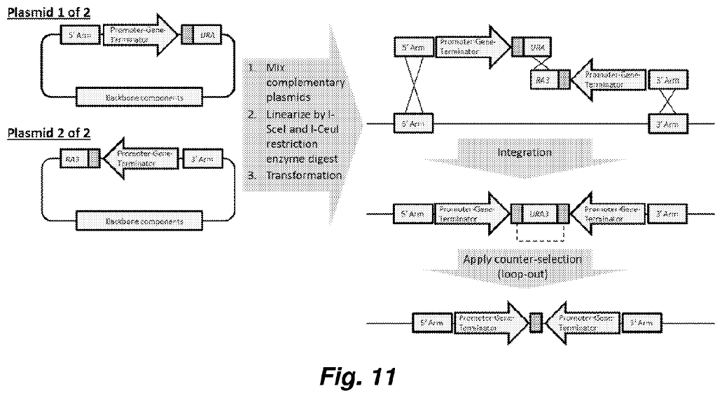

We conducted a search of metabolism [1] to identify enzymes that enable a metabolic pathway to produce cystathionine in industrial host organisms. To engineer production of cystathionine in an industrial microorganism requires genetic engineering tools and methods to manipulate DNA sequences (see FIGS. 11-14). Then, microbial metabolism is systematically reengineered to produce cystathionine, including in industrial hosts for which not all biochemical reactions or modes of metabolic regulation have been characterized, by iterative high-throughput (HTP) strain engineering using single-gene and multiple-gene modifications (see U.S. Patent Publication No. US20170159045A1 for a description of methods of HTP strain engineering, which is incorporated by reference herein for this description; see also International Patent Publication No. WO 2018203947, entitled “Engineered biosynthetic pathways for production of tyramine by fermentation”).

Plasmid/DNA Design

All strains tested for this work were transformed with plasmid DNA designed using proprietary software. Plasmid designs were specific to each of the host organisms engineered in this work. The plasmid DNA was physically constructed by a standard DNA assembly method. This plasmid DNA was then used to integrate metabolic pathway inserts by one of two host-specific methods, each described below.

C. glutamicum and B. subtilis Pathway Integration

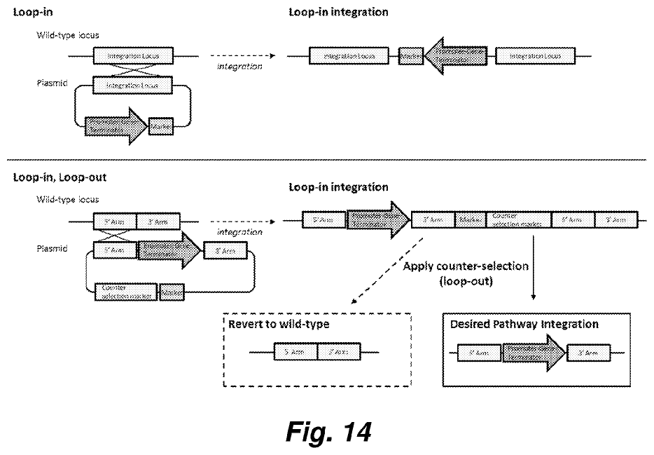

A “loop-in, single-crossover” genomic integration strategy has been developed to engineer C. glutamicum and B. subtilis strains. FIG. 14 illustrates genomic integration of loop-in only and loop-in/loop-out constructs and verification of correct integration via colony PCR. Loop-in only constructs (shown under the heading “Loop-in”) contained a single 2-kb homology arm (denoted as “integration locus”), a positive selection marker (denoted as “Marker”)), and gene(s) of interest (denoted as “promoter-gene-terminator”). A single crossover event integrated the plasmid into the C. glutamicum or B. subtilis chromosome. Integration events are stably maintained in the genome by growth in the presence of antibiotic (25 μg/mlkanamycin). Correct genomic integration in colonies derived from loop-in integration were confirmed by colony PCR with UF/IR and DR/IF PCR primers.

Loop-in, loop-out constructs (shown under the heading “Loop-in, loop-out) contained two 2-kb homology arms (5′ and 3′ arms), gene(s) of interest (arrows), a positive selection marker (denoted “Marker”), and a counter-selection marker. Similar to “loop-in” only constructs, a single crossover event integrated the plasmid into the chromosome. Note: only one of two possible integrations is shown here. Correct genomic integration was confirmed by colony PCR and counter-selection was applied so that the plasmid backbone and counter-selection marker could be excised. This results in one of two possibilities: reversion to wild-type (lower left box) or the desired pathway integration (lower right box). Again, correct genomic loop-out is confirmed by colony PCR. (Abbreviations: Primers: UF=upstream forward, DR=downstream reverse, IR=internal reverse, IF=internal forward.)

S. cerevisiae Pathway Integration

A “split-marker, double-crossover” genomic integration strategy has been developed to engineer S. cerevisiae strains. FIG. 11 illustrates genomic integration of complementary, split-marker plasmids and verification of correct genomic integration via colony PCR in S. cerevisiae. Two plasmids with complementary 5′ and 3′ homology arms and overlapping halves of a URA3 selectable marker (direct repeats shown by the hashed bars) were digested with meganucleases and transformed as linear fragments. A triple-crossover event integrated the desired heterologous genes into the targeted locus and re-constituted the full URA3 gene. Colonies derived from this integration event were assayed using two 3-primer reactions to confirm both the 5′ and 3′ junctions (UF/IF/wt-R and DR/IF/wt-F). For strains in which further engineering is desired, the strains can be plated on 5-FOA plates to select for the removal of URA3, leaving behind a small single copy of the original direct repeat. This genomic integration strategy can be used for gene knock-out, gene knock-in, and promoter titration in the same workflow.

Cell Culture

The workflow established for S. cerevisiae involved a hit-picking step that consolidated successfully built strains using an automated workflow that randomized strains across the plate. For each strain that was successfully built, up to four replicates were tested from distinct colonies to test colony-to-colony variation and other process variation. If fewer than four colonies were obtained, the existing colonies were replicated so that at least four wells were tested from each desired genotype.

The colonies were consolidated into 96-well plates with selective medium (SD-ura for S. cerevisiae) and cultivated for two days until saturation and then frozen with 16.6% glycerol at −80° C. for storage. The frozen glycerol stocks were then used to inoculate a seed stage in minimal media with a low level of amino acids to help with growth and recovery from freezing. The seed plates were grown at 30° C. for 1-2 days. The seed plates were then used to inoculate a main cultivation plate with minimal medium and grown for 48-88 hours. Plates were removed at the desired time points and tested for cell density (OD600), viability and glucose, supernatant samples stored for LC-MS analysis for product of interest.

Cell Density

Cell density was measured using a spectrophotometric assay detecting absorbance of each well at 600 nm. Robotics were used to transfer fixed amounts of culture from each cultivation plate into an assay plate, followed by mixing with 175 mM sodium phosphate (pH 7.0) to generate a 10-fold dilution. The assay plates were measured using a Tecan M1000 spectrophotometer and assay data uploaded to a LIMS database. A non-inoculated control was used to subtract background absorbance. Cell growth was monitored by inoculating multiple plates at each stage, and then sacrificing an entire plate at each time point.

To minimize settling of cells while handling large number of plates (which could result in a non-representative sample during measurement) each plate was shaken for 10-15 seconds before each read. Wide variations in cell density within a plate may also lead to absorbance measurements outside of the linear range of detection, resulting in underestimate of higher OD cultures. In general, the tested strains so far have not varied significantly enough for this be a concern.

Liquid-Solid Separation

To harvest extracellular samples for analysis by LC-MS, liquid and solid phases were separated via centrifugation. Cultivation plates were centrifuged at 2000 rpm for 4 minutes, and the supernatant was transferred to destination plates using robotics. 75 μL of supernatant was transferred to each plate, with one stored at 4° C., and the second stored at 80° C. for long-term storage.

First-Round Genetic Engineering Results in Corynebacteria glutamicum and Saccharomyces cerevisiae

Strains were designed and constructed to test enzymes in the pathway upstream of cystathionine (to glucose) via homoserine (see Table 1).

First-round genetic engineering results are shown in FIG. 2 (C. glutamicum) and 3 (S. cerevisiae). In C. glutamicum, successfully constructed strains constitutively expressed aspartate aminotransferase (EC 2.6.1.1), aspartate kinase (EC 2.7.2.4), aspartate semi-aldehyde dehydrogenase (EC 1.2.1.11), homoserine dehydrogenase (EC 1.1.1.3), homoserine O-succinyltransferase (EC 2.3.1.46), O-succinylhomoserine(thiol)-lyase (cystathionine gamma synthase) (EC 2.5.1.48), and cystathionine beta synthase (EC 4.2.1.22) (see Table 1). However, none of the strains achieved a cystathionine titer significantly greater than the wildtype strain.

In S. cerevisiae, the following enzymes were each expressed from a constitutive promoter: aspartate aminotransferase (EC 2.6.1.1), aspartate kinase (EC 2.7.2.4), aspartate semi-aldehyde dehydrogenase (EC 1.2.1.11), homoserine dehydrogenase (EC 1.1.1.3), homoserine O-succinyltransferase (EC 2.3.1.46), and O-succinylhomoserine(thiol)-lyase (cystathionine gamma synthase) (EC 2.5.1.48). In addition, a S. cerevisiae strain was designed and constructed to test expression of cystathionine beta-synthase (EC 4.2.1.22), which functions in the direction from homocysteine to cystathionine. The highest titer achieved was 48 microgram/L, from the strain expressing an additional copy of the S. cerevisiae cystathionine beta-synthase (UniProt ID N1P5Z1) from a constitutive promoter (here, “additional copy” refers to a gene in addition to the native gene).

TABLE 1
First-round genetic engineering results in Corynebacteria glutamicum and Saccharomyces cerevisiae

| | | | | | | |
|---|---|---|---|---|---|---|
| | | E1 | Enzyme 1 - | | Enzyme 1 - | E1 Codon |
| | Titer | Uniprot | activity | Enzyme 1 - | source | Optimization |
| Strain name | (μg/L) | ID | name | Modifications | organism | Abbrev. |

Corynebacterium glutamicum

| | | | | | | |
|---|---|---|---|---|---|---|
| CgCYTHIO_01 | 242.8 | Q8NTR2 | aspartate transaminase | 6 residue extension | Corynebacterium glutamicum | native |
| | | | | N-terminal | ATCC 13032 | |
| | | | | truncation MRRYAV. | | |
| | | | | Mutations include: | | |
| | | | | N177D, T198S, | | |
| | | | | A207T, L271M, | | |
| | | | | T281S, D332N, | | |
| | | | | N426S | | |
| CgCYTHIO_02 | 195.5 | N1P7Q4 | aspartate transaminase | | Saccharomyces cerevisiae | native |
| | | | | | CEN.PK113-7D | |
| CgCYTHIO_03 | 303.3 | N1NZ14 | aspartate transaminase | | Saccharomyces cerevisiae | native |
| | | | | | CEN.PK113-7D | |
| CgCYTHIO_04 | 268.8 | P26512 | aspartate kinase | A279T, S317A | Corynebacterium glutamicum | native |
| | | | | | ATCC 13032 | |
| CgCYTHIO_05 | 164.4 | N1P4U6 | aspartate kinase | | Saccharomyces cerevisiae | native |
| | | | | | CEN.PK113-7D | |
| CgCYTHIO_06 | 102.7 | P0C1D8 | aspartate semialdehyde | D66G, S202F, | Corynebacterium glutamicum | native |
| | | | dehydrogenase | R234H, D272E, | ATCC 13032 | |
| | | | | K285E | | |

Saccharomyces cerevisiae

| | | | | | | |
|---|---|---|---|---|---|---|
| ScCYTHIO_01 | 15.4 | Q8NTR2 | aspartate transaminase | 6 residue extension | Corynebacterium glutamicum | native |
| | | | | N-terminal | ATCC 13032 | |
| | | | | truncation MRRYAV. | | |
| | | | | Mutations include: | | |
| | | | | N177D, T198S, | | |
| | | | | A207T, L271M, | | |
| | | | | T281S, D332N, | | |
| | | | | N426S | | |
| ScCYTHIO_02 | 16.9 | N1P7Q4 | aspartate transaminase | | Saccharomyces cerevisiae | native |
| | | | | | CEN.PK113-7D | |
| ScCYTHIO_03 | 13.4 | N1NZ14 | aspartate transaminase | | Saccharomyces cerevisiae | native |
| | | | | | CEN.PK113-7D | |
| ScCYTHIO_05 | 21.0 | N1P4U6 | aspartate kinase | | Saccharomyces cerevisiae | native |
| | | | | | CEN.PK113-7D | |
| ScCYTHIO_06 | 15.0 | P0C1D8 | aspartate semialdehyde | D66G, S202F, | Corynebacterium glutamicum | native |
| | | | dehydrogenase | R234H, D272E, | ATCC 13032 | |
| | | | | K285E | | |
| ScCYTHIO_08 | 13.7 | P08499 | homoserine dehydrogenase | V104I, T116I, | Corynebacterium glutamicum | native |
| | | | | G148A | ATCC 13032 | |
| ScCYTHIO_09 | 15.7 | N1P1T8 | homoserine dehydrogenase | | Saccharomyces cerevisiae | native |
| | | | | | CEN.PK113-7D | |
| ScCYTHIO_10 | 14.6 | P07623 | homoserine O- | | Escherichia coli K12 | native |
| | | | succinyltransferase | | | |
| ScCYTHIO_11 | 12.1 | P00935 | O-succinylhomoserine(thiol)- | | Escherichia coli K12 | native |
| | | | lyase | | | |
| ScCYTHIO_12 | 47.5 | N1P5Z1 | cystathionine beta synthase | | Saccharomyces cerevisiae | native |
| | | | | | (strain CEN.PK113-7D) (Baker's yeast) | |

Second-Round Genetic Engineering Results in Saccharomyces cerevisiae

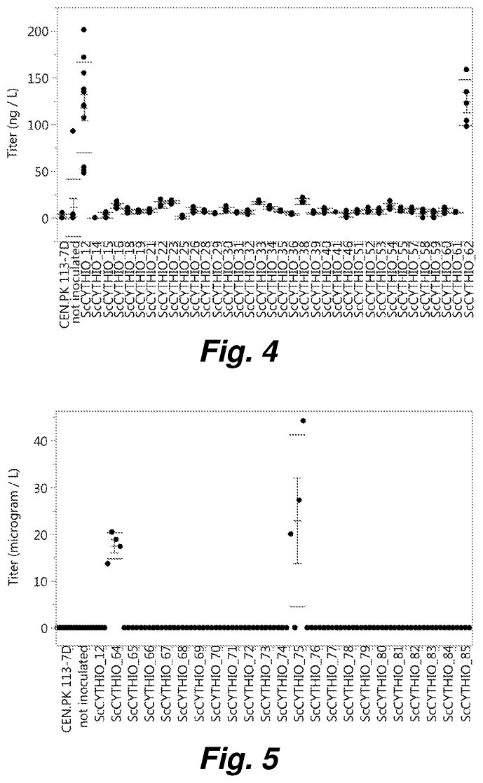

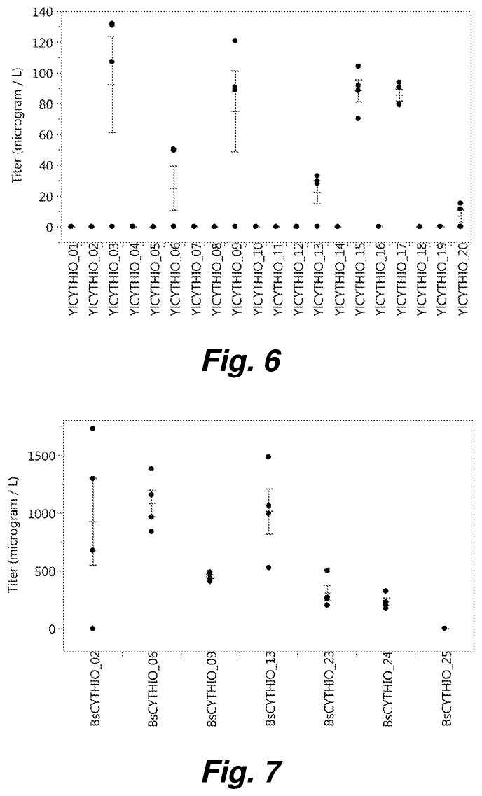

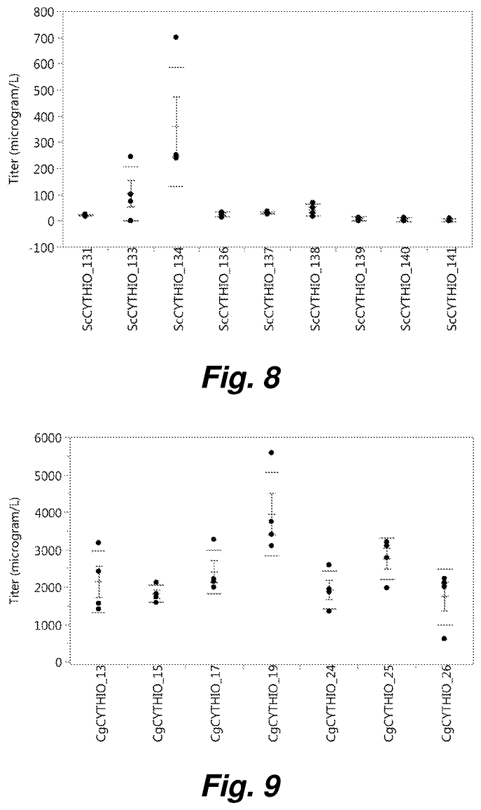

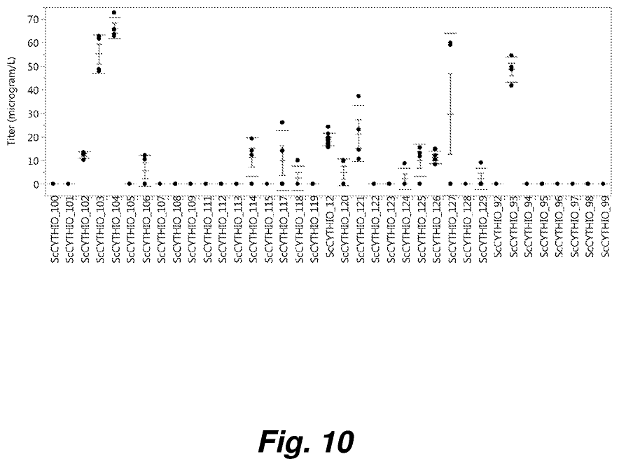

In Saccharomyces cerevisiae, strains were designed and constructed to test additional upstream cystathionine pathway enzymes in a second round of genetic engineering (Table 2). Each integrating plasmid was designed to constitutively express three enzymes in a strain selected from the list: aspartate transaminase (EC 2.6.1.1), aspartate-semialdehyde dehydrogenase (EC 1.2.1.11), aspartate kinase (EC 2.7.2.4), homoserine dehydrogenase (EC 1.1.1.3), cystathionine gamma-synthase (EC 2.5.1.48), and malate dehydrogenase (EC 1.1.1.37). None of the strains produced improved titer. (See FIG. 4.)

In addition the enzymes below, the Saccharomyces cerevisiae strains also contain cystathionine beta-synthase (UniProt ID N1P5Z1).

TABLE 2
Second-round genetic engineering results in Saccharomyces cerevisiae

| | | E1 | Enzyme 1 - | Enzyme 1 - | E1 Codon | E2 | Enzyme 2 - |
|---|---|---|---|---|---|---|---|
| Strain | Titer | Uniprot | activity | source | Optimization | Uniprot | activity |
| name | (μg/L) | ID | name | organism | Abbrev. | ID | name |
| ScCYTHIO_14 | 0.0 | Q8NTR2 | Aspartate | Corynebacterium | modified | Q8NN33 | Malate |
| | | | transaminase | glutamicum | codon | | dehydrogenase |
| | | | | ATCC 13032 | usage for | | |
| | | | | | Cg and Sc | | |
| ScCYTHIO_15 | 2.8 | P0C1D8 | Aspartate- | Corynebacterium | modified | P08499 | Homoserine |
| | | | semialdehyde | glutamicum | codon | | dehydrogenase |
| | | | dihydrogenase | ATCC 13032 | usage for | | (HDH) |
| | | | (ASA | | Cg and Sc | | |
| | | | dihydrogenase) | | | | |
| | | | (ASADH) | | | | |
| ScCYTHIO_16 | 13.1 | P26512 | aspartate | Corynebacterium | Corynebacterium | Q01802 | Aspartate |
| | | | kinase activity | glutamicum | glutamicum | | transaminase |
| ScCYTHIO_18 | 7.2 | P08499 | Homoserine | Corynebacterium | modified | P08499 | Homoserine |
| | | | dihydrogenase | glutamicum | codon | | dehydrogenase |
| | | | (HDH) | ATCC 13032 | usage for | | (HDH) |
| | | | | | Cg and Sc | | |
| ScCYTHIO_19 | 6.5 | P0C1D8 | Aspartate- | Corynebacterium | modified | P0C1D8 | Aspartate- |
| | | | semialdehyde | glutamicum | codon | | semialdehyde |
| | | | dihydrogenase | ATCC 13032 | usage for | | dehydrogenase |
| | | | (ASA | | Cg and Sc | | (ASA |
| | | | dihydrogenase) | | | | dehydrogenase) |
| | | | (ASADH) | | | | (ASADH) |
| ScCYTHIO_21 | 7.6 | Q01802 | Aspartate | Saccharomyces | modified | Q12128 | Malate |
| | | | transaminase | cerevisiae | codon | | dehydrogenase |
| | | | | S288c | usage for | | |
| | | | | | Cg and Sc | | |
| ScCYTHIO_22 | 14.4 | P26512 | aspartate | Corynebacterium | Corynebacterium | P31116 | Homoserine |
| | | | kinase activity | glutamicum | glutamicum | | dehydrogenase |
| | | | | | | | (HDH) |
| ScCYTHIO_23 | 17.0 | P31116 | Homoserine | Saccharomyces | modified | Q12128 | Malate |
| | | | dihydrogenase | cerevisiae | codon | | dehydrogenase |
| | | | (HDH) | S288c | usage for | | |
| | | | | | Cg and Sc | | |
| ScCYTHIO_25 | 0.7 | P31373 | cystathionine | Saccharomyces | modified | P47164 | cystathionine |
| | | | gamma- | cerevisiae | codon | | gamma- |
| | | | synthase | S288c | usage for | | synthase |
| | | | | | Cg and Sc | | |
| ScCYTHIO_26 | 8.1 | P31116 | Homoserine | Saccharomyces | modified | P31116 | Homoserine |
| | | | dihydrogenase | cerevisiae | codon | | dehydrogenase |
| | | | (HDH) | S288c | usage for | | (HDH) |
| | | | | | Cg and Sc | | |
| ScCYTHIO_28 | 6.3 | P26512 | aspartate | Corynebacterium | Corynebacterium | Q01802 | Aspartate |
| | | | kinase activity | glutamicum | glutamicum | | transaminase |
| ScCYTHIO_29 | 4.4 | Q8NN33 | Malate | Corynebacterium | modified | Q8NN33 | Malate |
| | | | dihydrogenase | glutamicum | codon | | dehydrogenase |
| | | | | ATCC 13032 | usage for | | |
| | | | | | Cg and Sc | | |
| ScCYTHIO_30 | 8.5 | P47164 | cystathionine | Saccharomyces | modified | P47164 | cystathionine |
| | | | gamma- | cerevisiae | codon | | gamma- |
| | | | synthase | S288c | usage for | | synthase |
| | | | | | Cg and Sc | | |
| ScCYTHIO_31 | 5.8 | P26512 | aspartate | Corynebacterium | Corynebacterium | P31116 | Homoserine |
| | | | kinase activity | glutamicum | glutamicum | | dehydrogenase |
| | | | | | | | (HDH) |
| ScCYTHIO_32 | 6.3 | P26512 | aspartate | Corynebacterium | Corynebacterium | Q12128 | Malate |
| | | | kinase activity | glutamicum | glutamicum | | dehydrogenase |
| ScCYTHIO_33 | 16.5 | P31116 | Homoserine | Saccharomyces | modified | P32801 | Aspartate- |
| | | | dihydrogenase | cerevisiae | codon | | semialdehyde |
| | | | (HDH) | S288c | usage for | | dehydrogenase |
| | | | | | Cg and Sc | | (ASA |
| | | | | | | | dihydrogenase) |
| | | | | | | | (ASADH) |
| ScCYTHIO_34 | 10.6 | Q01802 | Aspartate | Saccharomyces | modified | P31116 | Homoserine |
| | | | transaminase | cerevisiae | codon | | dehydrogenase |
| | | | | S288c | usage for | | (HDH) |
| | | | | | Cg and Sc | | |
| ScCYTHIO_35 | 7.0 | P26512 | aspartate | Corynebacterium | Corynebacterium | P31116 | Homoserine |
| | | | kinase activity | glutamicum | glutamicum | | dehydrogenase |
| | | | | | | | (HDH) |
| ScCYTHIO_36 | 4.1 | P0C1D8 | Aspartate- | Corynebacterium | modified | P08499 | Homoserine |
| | | | semialdehyde | glutamicum | codon | | dehydrogenase |
| | | | dihydrogenase | ATCC 13032 | usage for | | (HDH) |
| | | | (ASA | | Cg and Sc | | |
| | | | dihydrogenase) | | | | |
| | | | (ASADH) | | | | |
| ScCYTHIO_38 | 17.8 | P26512 | aspartate | Corynebacterium | Corynebacterium | Q12128 | Malate |
| | | | kinase activity | glutamicum | glutamicum | | dehydrogenase |
| ScCYTHIO_39 | 5.5 | P08499 | Homoserine | Corynebacterium | modified | Q8NTR2 | Aspartate |
| | | | dihydrogenase | glutamicum | codon | | transaminase |
| | | | (HDH) | ATCC 13032 | usage for | | |
| | | | | | Cg and Sc | | |
| ScCYTHIO_40 | 7.9 | Q8NTR2 | Aspartate | Corynebacterium | modified | Q8NTR2 | Aspartate |
| | | | transaminase | glutamicum | codon | | transaminase |
| | | | | ATCC 13032 | usage for | | |
| | | | | | Cg and Sc | | |
| ScCYTHIO_41 | 5.8 | P26512 | Aspartokinase | Corynebacterium | modified | P26512 | Aspartokinase |
| | | | | glutamicum | codon | | |
| | | | | ATCC 13032 | usage for | | |
| | | | | | Cg and Sc | | |
| ScCYTHIO_46 | 3.7 | P31373 | cystathionine | Saccharomyces | modified | P26512 | aspartate |
| | | | gamma- | cerevisiae | codon | | kinase activity |
| | | | synthase | S288c | usage for | | |
| | | | | | Cg and Sc | | |
| ScCYTHIO_51 | 6.8 | Q01802 | Aspartate | Saccharomyces | modified | Q01802 | Aspartate |
| | | | transaminase | cerevisiae | codon | | transaminase |
| | | | | S288c | usage for | | |
| | | | | | Cg and Sc | | |
| ScCYTHIO_52 | 6.9 | P0C1D8 | Aspartate- | Corynebacterium | modified | Q8NTR2 | Aspartate |
| | | | semialdehyde | glutamicum | codon | | transaminase |
| | | | dihydrogenase | ATCC 13032 | usage for | | |
| | | | (ASA | | Cg and Sc | | |
| | | | dihydrogenase) | | | | |
| | | | (ASADH) | | | | |
| ScCYTHIO_53 | 6.9 | P0C1D8 | Aspartate- | Corynebacterium | modified | Q8NN33 | Malate |
| | | | semialdehyde | glutamicum | codon | | dehydrogenase |
| | | | dihydrogenase | ATCC 13032 | usage for | | |
| | | | (ASA | | Cg and Sc | | |
| | | | dihydrogenase) | | | | |
| | | | (ASADH) | | | | |
| ScCYTHIO_54 | 12.5 | P10869 | Aspartokinase | Saccharomyces | modified | P10869 | Aspartokinase |
| | | | | cerevisiae | codon | | |
| | | | | S288c | usage for | | |
| | | | | | Cg and Sc | | |
| ScCYTHIO_55 | 7.7 | P0C1D8 | Aspartate- | Corynebacterium | modified | P08499 | Homoserine |
| | | | semialdehyde | glutamicum | codon | | dehydrogenase |
| | | | dihydrogenase | ATCC 13032 | usage for | | (HDH) |
| | | | (ASA | | Cg and Sc | | |
| | | | dihydrogenase) | | | | |
| | | | (ASADH) | | | | |
| ScCYTHIO_57 | 7.9 | P08499 | Homoserine | Corynebacterium | modified | P26512 | aspartate |
| | | | dihydrogenase | glutamicum | codon | | kinase activity |
| | | | (HDH) | ATCC 13032 | usage for | | |
| | | | | | Cg and Sc | | |
| ScCYTHIO_58 | 5.9 | P26512 | aspartate | Corynebacterium | Corynebacterium | Q01802 | Aspartate |
| | | | kinase activity | glutamicum | glutamicum | | transaminase |
| ScCYTHIO_59 | 4.8 | Q8NTR2 | Aspartate | Corynebacterium | modified | Q8NN33 | Malate |
| | | | transaminase | glutamicum | codon | | dehydrogenase |
| | | | | ATCC 13032 | usage for | | |
| | | | | | Cg and Sc | | |
| ScCYTHIO_60 | 7.8 | Q01802 | Aspartate | Saccharomyces | modified | Q12128 | Malate |
| | | | transaminase | cerevisiae | codon | | dehydrogenase |
| | | | | S288c | usage for | | |
| | | | | | Cg and Sc | | |
| ScCYTHIO_61 | 5.9 | P08499 | Homoserine | Corynebacterium | modified | Q8NTR2 | Aspartate |
| | | | dihydrogenase | glutamicum | codon | | transaminase |
| | | | (HDH) | ATCC 13032 | usage for | | |
| | | | | | Cg and Sc | | |
| ScCYTHIO_62 | 123.5 | P32801 | Aspartate- | Saccharomyces | modified | P32801 | Aspartate- |
| | | | semialdehyde | cerevisiae | codon | | semialdehyde |
| | | | dihydrogenase | S288c | usage for | | dehydrogenase |
| | | | (ASA | | Cg and Sc | | (ASA |
| | | | dihydrogenase) | | | | dihydrogenase) |
| | | | (ASADH) | | | | (ASADH) |

| | Enzyme 2 - | E2 Codon | E3 | Enzyme 3 - | Enzyme 3 - | E3 Codon |
|---|---|---|---|---|---|---|
| Strain | source | Optimization | Uniprot | activity | source | Optimization |
| name | organism | Abbrev. | ID | name | organism | Abbrev. |
| ScCYTHIO_14 | Corynebacterium | modified | P26512 | aspartate kinase | Corynebacterium | native |
| | glutamicum | codon | | activity | glutamicum | |
| | ATCC 13032 | usage for | | | | |
| | | Cg and Sc | | | | |
| ScCYTHIO_15 | Corynebacterium | modified | P26512 | aspartate kinase | Corynebacterium | native |
| | glutamicum | codon | | activity | glutamicum | |
| | ATCC 13032 | usage for | | | | |
| | | Cg and Sc | | | | |
| ScCYTHIO_16 | Saccharomyces | modified | P32801 | Aspartate- | Saccharomyces | modified |
| | cerevisiae | codon | | semialdehyde | cerevisiae | codon |
| | S288c | usage for | | dehydrogenase | S288c | usage for |
| | | Cg and Sc | | (ASA | | Cg and Sc |
| | | | | dehydrogenase) | | |
| | | | | (ASADH) | | |
| ScCYTHIO_18 | Corynebacterium | modified | P08499 | Homoserine | Corynebacterium | modified |
| | glutamicum | codon | | dehydrogenase | glutamicum | codon |
| | ATCC 13032 | usage for | | (HDH) | ATCC 13032 | usage for |
| | | Cg and Sc | | | | Cg and Sc |
| ScCYTHIO_19 | Corynebacterium | modified | P0C1D8 | Aspartate- | Corynebacterium | modified |
| | glutamicum | codon | | semialdehyde | glutamicum | codon |
| | ATCC 13032 | usage for | | dehydrogenase | ATCC 13032 | usage for |
| | | Cg and Sc | | (ASA | | Cg and Sc |
| | | | | dehydrogenase) | | |
| | | | | (ASADH) | | |
| ScCYTHIO_21 | Saccharomyces | modified | P32801 | Aspartate- | Saccharomyces | modified |
| | cerevisiae | codon | | semialdehyde | cerevisiae | codon |
| | S288c | usage for | | dehydrogenase | S288c | usage for |
| | | Cg and Sc | | (ASA | | Cg and Sc |
| | | | | dehydrogenase) | | |
| | | | | (ASADH) | | |
| ScCYTHIO_22 | Saccharomyces | modified | P32801 | Aspartate- | Saccharomyces | modified |
| | cerevisiae | codon | | semialdehyde | cerevisiae | codon |
| | S288c | usage for | | dehydrogenase | S288c | usage for |
| | | Cg and Sc | | (ASA | | Cg and Sc |
| | | | | dehydrogenase) | | |
| | | | | (ASADH) | | |
| ScCYTHIO_23 | Saccharomyces | modified | P32801 | Aspartate- | Saccharomyces | modified |
| | cerevisiae | codon | | semialdehyde | cerevisiae | codon |
| | S288c | usage for | | dehydrogenase | S288c | usage for |
| | | Cg and Sc | | (ASA | | Cg and Sc |
| | | | | dehydrogenase) | | |
| | | | | (ASADH) | | |
| ScCYTHIO_25 | Saccharomyces | modified | P47164 | cystathionine | Saccharomyces | modified |
| | cerevisiae | codon | | gamma- | cerevisiae | codon |
| | S288c | usage for | | synthase | S288c | usage for |
| | | Cg and Sc | | | | Cg and Sc |
| ScCYTHIO_26 | Saccharomyces | modified | P31116 | Homoserine | Saccharomyces | modified |
| | cerevisiae | codon | | dehydrogenase | cerevisiae | codon |
| | S288c | usage for | | (HDH) | S288c | usage for |
| | | Cg and Sc | | | | Cg and Sc |
| ScCYTHIO_28 | Saccharomyces | modified | Q12128 | Malate | Saccharomyces | modified |
| | cerevisiae | codon | | dehydrogenase | cerevisiae | codon |
| | S288c | usage for | | | S288c | usage for |
| | | Cg and Sc | | | | Cg and Sc |
| ScCYTHIO_29 | Corynebacterium | modified | Q8NN33 | Malate | Corynebacterium | modified |
| | glutamicum | codon | | dehydrogenase | glutamicum | codon |
| | ATCC 13032 | usage for | | | ATCC 13032 | usage for |
| | | Cg and Sc | | | | Cg and Sc |
| ScCYTHIO_30 | Saccharomyces | modified | P47164 | cystathionine | Saccharomyces | modified |
| | cerevisiae | codon | | gamma- | cerevisiae | codon |
| | S288c | usage for | | synthase | S288c | usage for |
| | | Cg and Sc | | | | Cg and Sc |
| ScCYTHIO_31 | Saccharomyces | modified | P31116 | Homoserine | Saccharomyces | modified |
| | cerevisiae | codon | | dehydrogenase | cerevisiae | codon |
| | S288c | usage for | | (HDH) | S288c | usage for |
| | | Cg and Sc | | | | Cg and Sc |
| ScCYTHIO_32 | Saccharomyces | modified | Q12128 | Malate | Saccharomyces | modified |
| | cerevisiae | codon | | dehydrogenase | cerevisiae | codon |
| | S288c | usage for | | | S288c | usage for |
| | | Cg and Sc | | | | Cg and Sc |
| ScCYTHIO_33 | Saccharomyces | modified | P32801 | Aspartate- | Saccharomyces | modified |
| | cerevisiae | codon | | semialdehyde | cerevisiae | codon |
| | S288c | usage for | | dehydrogenase | S288c | usage for |
| | | Cg and Sc | | (ASA | | Cg and Sc |
| | | | | dehydrogenase) | | |
| | | | | (ASADH) | | |
| ScCYTHIO_34 | Saccharomyces | modified | P32801 | Aspartate- | Saccharomyces | modified |
| | cerevisiae | codon | | semialdehyde | cerevisiae | codon |
| | S288c | usage for | | dehydrogenase | S288c | usage for |
| | | Cg and Sc | | (ASA | | Cg and Sc |
| | | | | dehydrogenase) | | |
| | | | | (ASADH) | | |
| ScCYTHIO_35 | Saccharomyces | modified | Q12128 | Malate | Saccharomyces | modified |
| | cerevisiae | codon | | dehydrogenase | cerevisiae | codon |
| | S288c | usage for | | | S288c | usage for |
| | | Cg and Sc | | | | Cg and Sc |
| ScCYTHIO_36 | Corynebacterium | modified | Q8NN33 | Malate | Corynebacterium | modified |
| | glutamicum | codon | | dehydrogenase | glutamicum | codon |
| | ATCC 13032 | usage for | | | ATCC 13032 | usage for |
| | | Cg and Sc | | | | Cg and Sc |
| ScCYTHIO_38 | Saccharomyces | modified | P32801 | Aspartate- | Saccharomyces | modified |
| | cerevisiae | codon | | semialdehyde | cerevisiae | codon |
| | S288c | usage for | | dehydrogenase | S288c | usage for |
| | | Cg and Sc | | (ASA | | Cg and Sc |
| | | | | dehydrogenase) | | |
| | | | | (ASADH) | | |
| ScCYTHIO_39 | Corynebacterium | modified | P26512 | aspartate kinase | Corynebacterium | native |
| | glutamicum | codon | | activity | glutamicum | |
| | ATCC 13032 | usage for | | | | |
| | | Cg and Sc | | | | |
| ScCYTHIO_40 | Corynebacterium | modified | Q8NTR2 | Aspartate | Corynebacterium | modified |
| | glutamicum | codon | | transaminase | glutamicum | codon |
| | ATCC 13032 | usage for | | | ATCC 13032 | usage for |
| | | Cg and Sc | | | | Cg and Sc |
| ScCYTHIO_41 | Corynebacterium | modified | P26512 | Aspartokinase | Corynebacterium | modified |
| | glutamicum | codon | | | glutamicum | codon |
| | ATCC 13032 | usage for | | | ATCC 13032 | usage for |
| | | Cg and Sc | | | | Cg and Sc |
| ScCYTHIO_46 | Corynebacterium | Corynebacterium | P31116 | Homoserine | Saccharomyces | modified |
| | glutamicum | glutamicum | | dehydrogenase | cerevisiae | codon |
| | | | | (HDH) | S288c | usage for |
| | | | | | | Cg and Sc |
| ScCYTHIO_51 | Saccharomyces | modified | Q01802 | Aspartate | Saccharomyces | modified |
| | cerevisiae | codon | | transaminase | cerevisiae | codon |
| | S288c | usage for | | | S288c | usage for |
| | | Cg and Sc | | | | Cg and Sc |
| ScCYTHIO_52 | Corynebacterium | modified | Q8NTR2 | Aspartate | Corynebacterium | modified |
| | glutamicum | codon | | transaminase | glutamicum | codon |
| | ATCC 13032 | usage for | | | ATCC 13032 | usage for |
| | | Cg and Sc | | | | Cg and Sc |
| ScCYTHIO_53 | Corynebacterium | modified | Q8NN33 | Malate | Corynebacterium | modified |
| | glutamicum | codon | | dehydrogenase | glutamicum | codon |
| | ATCC 13032 | usage for | | | ATCC 13032 | usage for |
| | | Cg and Sc | | | | Cg and Sc |
| ScCYTHIO_54 | Saccharomyces | modified | P10869 | Aspartokinase | Saccharomyces | modified |
| | cerevisiae | codon | | | cerevisiae | codon |
| | S288c | usage for | | | S288c | usage for |
| | | Cg and Sc | | | | Cg and Sc |
| ScCYTHIO_55 | Corynebacterium | modified | Q8NTR2 | Aspartate | Corynebacterium | modified |
| | glutamicum | codon | | transaminase | glutamicum | codon |
| | ATCC 13032 | usage for | | | ATCC 13032 | usage for |
| | | Cg and Sc | | | | Cg and Sc |
| ScCYTHIO_57 | Corynebacterium | Corynebacterium | P26512 | aspartate kinase | Corynebacterium | native |
| | glutamicum | glutamicum | | activity | glutamicum | |
| ScCYTHIO_58 | Saccharomyces | modified | Q01802 | Aspartate | Saccharomyces | modified |
| | cerevisiae | codon | | transaminase | cerevisiae | codon |
| | S288c | usage for | | | S288c | usage for |
| | | Cg and Sc | | | | Cg and Sc |
| ScCYTHIO_59 | Corynebacterium | modified | Q8NN33 | Malate | Corynebacterium | modified |
| | glutamicum | codon | | dehydrogenase | glutamicum | codon |
| | ATCC 13032 | usage for | | | ATCC 13032 | usage for |
| | | Cg and Sc | | | | Cg and Sc |
| ScCYTHIO_60 | Saccharomyces | modified | Q12128 | Malate | Saccharomyces | modified |
| | cerevisiae | codon | | dehydrogenase | cerevisiae | codon |
| | S288c | usage for | | | S288c | usage for |
| | | Cg and Sc | | | | Cg and Sc |
| ScCYTHIO_61 | Corynebacterium | modified | Q8NN33 | Malate | Corynebacterium | modified |
| | glutamicum | codon | | dehydrogenase | glutamicum | codon |
| | ATCC 13032 | usage for | | | ATCC 13032 | usage for |
| | | Cg and Sc | | | | Cg and Sc |
| ScCYTHIO_62 | Saccharomyces | modified | P32801 | Aspartate- | Saccharomyces | modified |
| | cerevisiae | codon | | semialdehyde | cerevisiae | codon |
| | S288c | usage for | | dehydrogenase | S288c | usage for |
| | | Cg and Sc | | (ASA | | Cg and Sc |
| | | | | dehydrogenase) | | |
| | | | | (ASADH) | | |

Third-Round Genetic Engineering Results in Saccharomyces cerevisiae

In Saccharomyces cerevisiae, strains were designed and constructed to test additional upstream cystathionine enzymes in a third round of genetic engineering (Table 3). Each integrating plasmid was designed to constitutively express 1-3 enzymes selected from the list: aspartate kinase (EC 2.7.2.4), feedback-deregulated aspartokinase

(EC 2.7.2.4), harboring either E250K or M318I; aspartate aminotransferase (EC 2.6.1.1); homoserine dehydrogenase (EC 1.1.1.3); feedback-deregulated homoserine dehydrogenase (EC 1.1.1.3), harboring the set of amino acid substitutions: V104I, T116I, G148A or the set of amino acid substitutions: A429L, K430S, P431L, V432L, V433L, K434R, A435Q, I436S, N437T, S438V and the deletion AA 439-445; aspartate-semialdehyde dehydrogenase (EC 1.2.1.11); feedback-deregulated aspartate semialdehyde dehydrogenase (EC 1.2.1.11), harboring the set of amino acid substitutions: D66G, S202F, R234H, D272E, K285E; feedback-deregulated homoserine transsuccinylase (EC 2.3.1.46), harboring R27C or I296S; feedback-deregulated phosphoenolpyruvate carboxylase (EC 4.1.1.31), harboring either N917G or D299N; feedback-deregulated pyruvate carboxylase (EC 6.4.1.1), harboring P458S; and malate dehydrogenase (EC 1.1.1.37). None of the strains produced improved titer. (See FIG. 5)

In addition the enzymes below, the Saccharomyces cerevisiae strains also contain cystathionine beta-synthase (UniProt ID N1P5Z1).

TABLE 3
Third-round genetic engineering results in Saccharomyces cerevisiae

| | | | | | | E1 | | |
|---|---|---|---|---|---|---|---|---|
| | | E1 | Enzyme 1 - | | Enzyme 1 - | Codon | E2 | Enzyme 2 - |
| Strain | Titer | Uniprot | activity | E1 | source | Optimization | Uniprot | activity |
| name | (μg/L) | ID | name | Modifications | organism | Abbrev. | ID | name |
| ScCYTHIO_64 | 17.56 | P08660 | Aspartokinase | E250K | Escherichia | modified | | |
| | | | III | | coli K12 | codon | | |
| | | | | | | usage | | |
| | | | | | | for Cg | | |
| | | | | | | and Sc | | |
| ScCYTHIO_65 | 0 | P23542 | aspartate | | Saccharomyces | modified | | |
| | | | aminotransferase | | cerevisiae | codon | | |
| | | | actvity | | S288c | usage | | |
| | | | | | | for Cg | | |
| | | | | | | and Sc | | |
| ScCYTHIO_66 | 0 | P08499 | homoserine | V104I, | Corynebacterium | native | | |
| | | | dihydrogenase | T116I, | glutamicum | | | |
| | | | | G148A | ATCC 13032 | | | |
| ScCYTHIO_67 | 0 | P0C1D8 | aspartate | D66G, | Corynebacterium | native | | |
| | | | semialdehyde | S202F, | glutamicum | | | |
| | | | dihydrogenase | R234H, | ATCC 13032 | | | |
| | | | | D272E, | | | | |
| | | | | K285E | | | | |
| ScCYTHIO_68 | 0 | P0C1D8 | aspartate | D66G, | Corynebacterium | native | P08499 | Homoserine |
| | | | semialdehyde | S202F, | glutamicum | | | dihydrogenase |
| | | | dihydrogenase | R234H, | ATCC 13032 | | | (HDH) |
| | | | | D272E, | | | | |
| | | | | K285E | | | | |
| ScCYTHIO_69 | 0 | P08499 | Homoserine | A429L, K430S, | Corynebacterium | modified | | |
| | | | dihydrogenase | P431L, V432L, | glutamicum | codon | | |
| | | | | V433L, K434R, | ATCC 13032 | usage | | |
| | | | | A435Q, I436S, | | for Cg | | |
| | | | | N437T, S438V, | | and Sc | | |
| | | | | delete AA | | | | |
| | | | | 439-445 | | | | |
| ScCYTHIO_70 | 0 | P0C1D8 | Aspartate- | | Corynebacterium | modified | P08499 | homoserine |
| | | | semialdehyde | | glutamicum | codon | | dihydrogenase |
| | | | dihydrogenase | | ATCC 13032 | usage | | |
| | | | (ASA | | | for Cg | | |
| | | | dihydrogenase) | | | and Sc | | |
| | | | (ASADH) | | | | | |
| ScCYTHIO_71 | 0 | P32801 | Aspartate- | | Saccharomyces | modified | P26512 | aspartate |
| | | | semialdehyde | | cerevisiae | codon | | kinase |
| | | | dihydrogenase | | S288c | usage | | activity |
| | | | (ASA | | | for Cg | | |
| | | | dihydrogenase) | | | and Sc | | |
| | | | (ASADH) | | | | | |
| ScCYTHIO_72 | 0 | P0C1D8 | aspartate | D66G, | Corynebacterium | native | P08499 | homoserine |
| | | | semialdehyde | S202F, | glutamicum | | | dihydrogenase |
| | | | dihydrogenase | R234H, | ATCC 13032 | | | |
| | | | | D272E, | | | | |
| | | | | K285E | | | | |
| ScCYTHIO_73 | 0 | P0C1D8 | aspartate | D66G, | Corynebacterium | native | P08499 | Homoserine |
| | | | semialdehyde | S202F, | glutamicum | | | dihydrogenase |
| | | | dehydrogenase- | R234H, | ATCC 13032 | | | (HDH) |
| | | | | D272E, | | | | |
| | | | | K285E | | | | |
| ScCYTHIO_74 | 0 | P07623 | Homoserine | R27C | Escherichia | modified | | |
| | | | transsuccinylase | | coli K12 | codon | | |
| | | | | | | usage | | |
| | | | | | | for Cg | | |
| | | | | | | and Sc | | |
| ScCYTHIO_75 | 22.86 | P08660 | Aspartokinase | M318I | Escherichia | modified | | |
| | | | III | | coli | codon | | |
| | | | | | (strain K12) | usage | | |
| | | | | | | for Cg | | |
| | | | | | | and Sc | | |
| ScCYTHIO_76 | 0 | P0C1D8 | aspartate | D66G, | Corynebacterium | native | P26512 | aspartate |
| | | | semialdehyde | S202F, | glutamicum | | | kinase |
| | | | dihydrogenase | R234H, | ATCC 13032 | | | activity |
| | | | | D272E, | | | | |
| | | | | K285E | | | | |
| ScCYTHIO_77 | 0 | P0C1D8 | Aspartate- | | Corynebacterium | modified | P26512 | aspartate |
| | | | semialdehyde | | glutamicum | codon | | kinase |
| | | | dihydrogenase | | ATCC 13032 | usage | | activity |
| | | | (ASA | | | for Cg | | |
| | | | dihydrogenase) | | | and Sc | | |
| | | | (ASADH) | | | | | |
| ScCYTHIO_78 | 0 | Q8NTR2 | Aspartate | | Corynebacterium | modified | P12880 | Phosphoenolpyruvate |
| | | | transaminase | | glutamicum | codon | | carboxylase |
| | | | | | ATCC 13032 | usage | | |
| | | | | | | for Cg | | |
| | | | | | | and Sc | | |
| ScCYTHIO_79 | 0 | P12880 | Phospho- | N917G | Corynebacterium | modified | | |
| | | | enolpyruvate | | glutamicum | codon | | |
| | | | carboxylase | | ATCC 13032 | usage | | |
| | | | | | | for Cg | | |
| | | | | | | and Sc | | |
| ScCYTHIO_80 | 0 | P07623 | Homoserine | I296S | Escherichia | modified | | |
| | | | transsuccinylase | | coli K12 | codon | | |
| | | | | | | usage | | |
| | | | | | | for Cg | | |
| | | | | | | and Sc | | |
| ScCYTHIO_81 | 0 | H7C7K2 | Pyruvate | P458S | Corynebacterium | modified | Q8NN33 | Malate |
| | | | carboxylase | | glutamicum | codon | | dihydrogenase |
| | | | | | ATCC 13032 | usage | | |
| | | | | | | for Cg | | |
| | | | | | | and Sc | | |
| ScCYTHIO_82 | 0 | Q8NN33 | Malate | | Corynebacterium | modified | P12880 | Phosphoenolpyruvate |
| | | | dihydrogenase | | glutamicum | codon | | carboxylase |
| | | | | | ATCC 13032 | usage | | |
| | | | | | | for Cg | | |
| | | | | | | and Sc | | |
| ScCYTHIO_83 | 0 | P23542 | aspartate | | Saccharomyces | modified | Q12128 | Malate |
| | | | aminotransferase | | cerevisiae | codon | | dihydrogenase |
| | | | activity | | S288c | usage | | |
| | | | | | | for Cg | | |
| | | | | | | and Sc | | |
| ScCYTHIO_84 | 0 | H7C7K2 | Pyruvate | P458S | Corynebacterium | modified | Q8NN33 | Malate |
| | | | carboxylase | | glutamicum | codon | | dihydrogenase |
| | | | | | ATCC 13032 | usage | | |
| | | | | | | for Cg | | |
| | | | | | | and Sc | | |
| ScCYTHIO_85 | 0 | P08499 | homoserine | V104I, | Corynebacterium | native | P26512 | aspartate |
| | | | dihydrogenase | T116I, | glutamicum | | | kinase |
| | | | | G148A | ATCC 13032 | | | activity |

| | | | E2 | | | | | E3 |
|---|---|---|---|---|---|---|---|---|
| | | Enzyme 2 - | Codon | E3 | Enzyme 3 | | Enzyme 3 - | Codon |
| Strain | E2 | source | Optimization | Uniprot | activity | E3 | source | Optimization |
| name | Modifications | organism | Abbrev. | ID | name | Modifications | organism | Abbrev. |
| ScCYTHIO_64 | | | | | | | | |
| ScCYTHIO_65 | | | | | | | | |
| ScCYTHIO_66 | | | | | | | | |
| ScCYTHIO_67 | | | | | | | | |
| ScCYTHIO_68 | | Corynebacterium | Modified | P26512 | Aspartate | | Corynebacterium | native |
| | | glutamicum | codon | | kinase | | glutamicum | |
| | | ATCC 13032 | usage | | activity | | ATCC 13032 | |
| | | | for Cg | | | | | |
| | | | and Sc | | | | | |
| ScCYTHIO_69 | | | | | | | | |
| ScCYTHIO_70 | V104I, | Corynebacterium | native | | | | | |
| | T116I, | glutamicum | | | | | | |
| | G148A | ATCC 13032 | | | | | | |
| ScCYTHIO_71 | | Corynebacterium | Modified | | | | | |
| | | glutamicum R | codon | | | | | |
| | | | usage | | | | | |
| | | | for Cg | | | | | |
| | | | and Sc | | | | | |
| ScCYTHIO_72 | V104I, | Corynebacterium | native | | | | | |
| | T116I, | glutamicum | | | | | | |
| | G148A | ATCC 13032 | | | | | | |
| ScCYTHIO_73 | | Corynebacterium | Modified | | | | | |
| | | glutamicum | codon | | | | | |
| | | ATCC 13032 | usage | | | | | |
| | | | for Cg | | | | | |
| | | | and Sc | | | | | |
| ScCYTHIO_74 | | | | | | | | |
| ScCYTHIO_75 | | | | | | | | |
| ScCYTHIO_76 | | Corynebacterium | Modified | | | | | |
| | | glutamicum R | codon | | | | | |
| | | | usage | | | | | |
| | | | for Cg | | | | | |
| | | | and Sc | | | | | |
| ScCYTHIO_77 | | Corynebacterium | Modified | | | | | |
| | | glutamicum R | codon | | | | | |
| | | | usage | | | | | |
| | | | for Cg | | | | | |
| | | | and Sc | | | | | |
| ScCYTHIO_78 | D299N | Corynebacterium | Modified | | | | | |
| | | glutamicum | codon | | | | | |
| | | ATCC 13032 | usage | | | | | |
| | | | for Cg | | | | | |
| | | | and Sc | | | | | |
| ScCYTHIO_79 | | | | | | | | |
| ScCYTHIO_80 | | | | | | | | |
| ScCYTHIO_81 | | Corynebacterium | Modified | P08499 | Homo- | V104I, | Corynebacterium | native |
| | | glutamicum | codon | | serine | T116I, | glutamicum | |
| | | ATCC 13032 | usage | | dehydrogenase | G148A | ATCC 13032 | |
| | | | for Cg | | | | | |
| | | | and Sc | | | | | |
| ScCYTHIO_82 | N917G | Corynebacterium | Modified | | | | | |
| | | glutamicum | codon | | | | | |
| | | ATCC 13032 | usage | | | | | |
| | | | for Cg | | | | | |
| | | | and Sc | | | | | |
| ScCYTHIO_83 | | Saccharomyces | Modified | | | | | |
| | | cerevisiae | codon | | | | | |
| | | S288c | usage | | | | | |
| | | | for Cg | | | | | |
| | | | and Sc | | | | | |
| ScCYTHIO_84 | | Corynebacterium | Modified | | | | | |
| | | glutamicum | codon | | | | | |
| | | ATCC 13032 | usage | | | | | |
| | | | for Cg | | | | | |
| | | | and Sc | | | | | |
| ScCYTHIO_85 | | Corynebacterium | Modified | | | | | |
| | | glutamicum R | codon | | | | | |
| | | | usage | | | | | |
| | | | for Cg | | | | | |
| | | | and Sc | | | | | |

### EXAMPLE 2

### Engineering to Improve Cystathionine Production

Cystathionine was further pursued in Saccharomyces cerevisiae: we designed plasmids to integrate additional copies of upstream pathway genes expressed by a strong constitutive promoter to avoid native regulation of a gene (Table 4). The designs described for S. cerevisiae are also generalized (below) for cystathionine production in each of Corynebacteria glutamicum, Bacillus subtillus and Yarrowia lipolytica, taking into account similarities and differences in sulfur incorporation by the transsulfuration and direct sulfhydrylation pathways in these host organisms (FIG. 1 and Table 6).

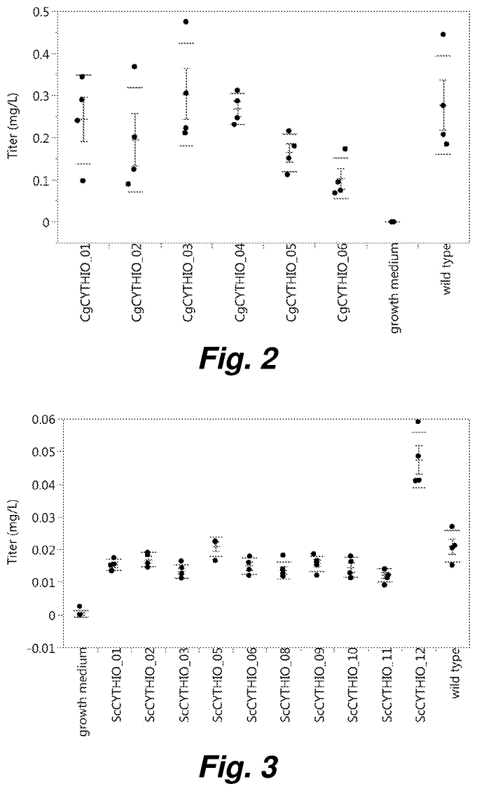

In S. cerevisiae cysteine is only produced through the transsulfuration pathway [2]. Cystathionine is degraded by cystathionine gamma lyase to produce cysteine. Expression of cystathionine beta-synthase improved production of cystathionine (FIG. 3 and Table 1). Cysteine is a substrate for cystathionine beta-synthase, therefore the strain contains a futile cycle that increased the cystathionine metabolite pool. To further improve cystathionine production, enzyme activities that degrade cystathionine were decreased or removed, and biosynthesis of cysteine by direct sulfhydrylation was installed. The approaches taken included the following:

Install and/or increase activity or expression of cysteine synthase (EC 2.5.1.47) in the host organism. Examples of this activity include E. coli cysteine synthase genes cysK and cysM and B. subtillus cysteine synthase genes cysK and ytkP. CysM can also use thiosulfate as a sulfur substrate, in addition to sulfide [12].

Decrease activity, expression, or eliminate cystathionine gamma lyase (EC 4.4.1.1) from the host organism (cys3 in S. cerevisiae or yrhB in B. subtillus). Ono et al. found that upon deletion of cys3, S. cerevisiae had increased intracellular cystathionine [10].

Decrease activity, expression, or eliminate cystathionine beta lyase (EC 4.4.1.8) from the host organism (STR3 and/or IRC7 in S. cerevisiae, Cg12309 in C. glutamicum, yjcJ in B. subtillus, and YALI0D00605g in Y. lipolytica).

Install and/or increase activity or expression of homocysteine synthase (EC 4.2.99.10) in the host organism (MET25 [also called MET17, MET15] from S. cerevisiae) which catalyzes the reaction of acetylated homoserine with the thiol sulfide (S²⁻) to produce L-homocysteine. In the absence of cystathionine beta lyase, homocysteine synthase provides the only route to L-homocysteine and L-methionine.

Production of cystathionine utilizes the biosynthetic precursors L-serine and L-homoserine. Strain genetic modifications that improve production of each of these amino acids was anticipated to improve production of cystathionine in all four hosts (S. cerevisiae, C. glutamicum, B. subtillus and Y. lipolytica).

Homoserine is derived from aspartate biosynthesis pathway, therefore installing a feedback-deregulated aspartokinase (EC 2.7.2.4), such as E. coli aspartokinase (UniProt ID P08660), harboring an amino acid substitution from the list: E250K, T344M, T352I, M318I, G323D, L325F, or S345L [13, 14] was anticipated to improve flux to cystathionine.

Strongly express a homoserine dehydrogenase (EC 1.1.1.3) from C. glutamicum (UniProt ID P08499), harboring the feedback-deregulation amino acid substitution G377E [15] or a C-terminal truncation that abolishes allosteric inhibition by L-threonine [16].

Serine is derived from the glycolysis intermediate 3-phosphoglycerate. Increased activity or expression of 3-phosphoglycerate dehydrogenase (EC 1.1.1.95), phosphoserine transaminase (EC 2.6.1.52), or phosphoserine phosphatase (EC 3.1.3.3) can improve the availability of serine and thereby improve production of cystathionine.

Either serine or homoserine can function as the sulfur acceptor for cystathionine synthase, and the activated form can be O-acetylated or O-succinylated.

Install and/or increase activity or expression of serine O-acetyltransferase (EC 2.3.1.30) in the host organism to provide the substrate O-acetylserine for cysteine synthase, e.g.: CysE from B.s subtillus, Cg12563 C. glutamicum or feedback-deregulated CysE from E. coli (UniProt P0A9D4), harboring the amino acid substitution M256W [17].

Install homoserine O-acetyltransferase (EC 2.3.1.31) in the host organism to provide the substrate O-acetylhomoserine for homocysteine synthase.

Install and/or increase activity or expression of homoserine O-succinyltransferase (EC 2.3.1.46) in the host organism to provide the substrate O-succinylhomoserine for homocysteine synthase, e.g.: metA from E. coli (UniProt ID P07623), harboring the amino acid substitution I296S, P298L or R27C [18], or an amino acid substitution from the list: Q96K, I124L I229Y and F247Y, to produce a thermos-stabilized homoserine 0-succinyltransferase [19].

Sulfur incorporation into cystathionine is engineered by installing or constitutively expressing cysteine synthase or homocysteine synthase (described above) in each host organism. Each enzyme uses sulfide (S²⁻) as the sulfur donor provided by the sulfate reduction pathway. To lower the metabolic burden of reducing sulfate to sulfide, thiosulfate can be used instead as the sulfur source to improve production of cystathionine, as it has been found to improve production of cysteine [20].

Increase activity or expression of ATP sulfurase (EC 2.7.7.4), APS kinase (EC 2.7.1.25), and/or PAPS reductase (EC 1.8.4.8) for incorporation of sulfate into cystathionine.

Increase activity or expression of sulfite reductase (EC 1.8.99.1) to improve incorporation of sulfate or thiosulfate into cystathionine.

Express an amino acid transporter such as S. cerevisiae AQR1 (YNL065W) [21, 22] to improve excretion.

For a selection of native enzymes, production of cystathionine can be improved when the activity becomes lower than the specific activity in an unmodified strain, or a wild type organism. The activity can be reduced to 50% or less, 30% or less, or 10% or less per microbial cell, as compared with that in the unmodified or wild-type strain. The activity can also be completely eliminated, such as through deletion of the gene. It is only necessary that the activity is lower than that in the wild-type strain or the unmodified strain, but further accumulation of cystathionine is desirably enhanced compared with these strains. We pursued modulating native gene expression to further improve cystathionine production. The gene targets for promoter changes were selected to redirect flux supply precursors to cystathionine or to diminish branching pathways that deplete cystathionine precursors. The approaches taken included the following:

Decrease activity or lower expression of homoserine kinase (EC 2.7.1.39), such as Thr1 in S. cerevisiae, by a promoter swap (PROSWP), since this enzyme utilizes serine.

Decrease activity or lower expression of threonine synthase (EC 4.2.3.1), such as Thr4 in S. cerevisiae, by a PROSWP, since this enzyme utilizes serine.

Decrease activity or lower expression of catabolic serine deaminase (EC 4.3.1.17), such as Chal in S. cerevisiae, by a PROSWP to improve cystathionine production in the host organism.

Decrease activity or lower expression of methionine synthase (EC 2.1.1.13) which consumes homocysteine to improve cystathionine production in the host organism.

Decrease activity or lower expression of glutathione synthase (EC 6.3.2.3) which consumes cysteine to improve cystathionine production in the host organism.

Decrease activity, lower expression, or eliminate L-cysteine desulfhydrase (EC 2.8.1.7) activity to improve cysteine availability to improve cystathionine production in the host organism. In C. glutamicum decrease expression of Cg11067, Cg11232, and/or Cg11561. In B. subtillus decrease expression of BSU27510 (iscS), BSU27880 (nifS), BSU29590 (iscS), an/or BSU32690 (sufS). In S. cerevisiae decrease expression of Nfslp. In Y. lipolytica decrease expression of YALI0C19041g [17, 20, 23-28].

Host Evaluation Results

All strain designs that expressed enzymes via genes that were codon-optimized for Y. lipolytica produced cystathionine, whereas for the same strain designs in which the enzymes were codon-optimized for the other host organisms, only 1 of 14 strain designs produced cystathionine.

The best-performing Y. lipolytica strain produced 92.5 microgram/L cystathionine and the expressed cystathionine beta-synthase from S. cerevisiae (UniProt ID N1P5Z1), cystathionine gamma-synthase from Bacillus paralicheniformis ATCC 9945a (UniProt ID R9TW27), and feedback-deregulated aspartokinase from S.s cerevisiae S288c (UniProt ID P10869), harboring the amino acid substitution G452D.

The best-performing B. subtillus strain produced 1.0 mg/L cystathionine and expressed cystathionine beta-synthase from S. cerevisiae (UniProt ID N1P5Z1), cystathionine gamma-synthase from B. paralicheniformis ATCC 9945a (UniProt ID R9TW27), and feedback-deregulated aspartokinase from S. cerevisiae S288c (UniProt ID P10869), harboring the amino acid substitution G452D.

The best-performing host evaluation design tested in S. cerevisiae produced 360 microgram/L and expressed cystathionine beta-synthase from S. cerevisiae (UniProt ID N1P5Z1), cystathionine gamma synthase from Escherichia coli K12 (UniProt ID P00935) and feedback-deregulated aspartokinase from S. cerevisiae S288c (UniProt ID P10869), harboring the amino acid substitution G452D.

The best performing C. glutamicum strain produced 4.0 mg/L and expressed cystathionine beta-synthase from S. cerevisiae (UniProt ID N1P5Z1), cystathionine gamma synthase from E. coli K12 (UniProt ID P00935), and aspartate aminotransferase from S. cerevisiae CEN.PK113-7D (UniProt ID N1NZ14).

In S. cerevisiae, 3 strains have improved cystathionine titer relative to the control ScCYSTHIO_12, which produced 19.1 microgram/L. The improved strains expressed the following enzymes:

1. Phosphoenolpyruvate carboxylase (EC 4.1.1.31) from E. coli K12 (UniProt ID P00864), aspartate aminotransferase from E. coli K12 (UniProt ID P00509), and bifunctional aspartokinase (EC 2.7.2.4)/homoserine dehydrogenase (EC 1.1.1.3) harboring the amino acid substitution S345F, which produced 55.2 microgram/L cystathionine;

2. Phosphoenolpyruvate carboxylase (EC 4.1.1.31) from E. coli K12 (UniProt ID P00864), aspartate aminotransferase from S. cerevisiae S288c (UniProt ID P23542), and bifunctional aspartokinase (EC 2.7.2.4)/homoserine dehydrogenase (EC 1.1.1.3), harboring the amino acid substitution S345F, which produced 66.1 microgram/L cystathionine; and

3. Sulfite reductase (EC 1.8.1.2) from S. cerevisiae S288c (UniProt ID P47169), sulfite reductase (EC 1.8.1.2) from S. cerevisiae S288c (UniProt ID P39692), and homocysteine/cysteine synthase (EC 2.5.1.47) from S. cerevisiae S288c (UniProt ID P06106), which produced 29.7 microgram/L cystathionine.

Yield Improvement

The yield of cystathionine can be improved by altering the cofactor specificity of cystathionine pathway enzymes to use NADH preferentially over NADPH. Several pathway enzymes use NADPH, including aspartate semi-aldehyde dehydrogenase and homoserine dehydrogenase. In order to meet pathway demand for NADPH, the pentose phosphate pathway must be used. The yield of cystathionine can be increased by altering the cofactor specificity of aspartate semi-aldehyde dehydrogenase to use NADH preferentially over NADPH. Mining of natural NADH-utilizing dehydrogenases has yielded enzymes such as aspartate semi-aldehyde dehydrogenase from Tistrella mobilis that use NADH [23]. The yield of cystathionine can be further enhanced by altering the cofactor specificity of homoserine dehydrogenase to use NADH preferentially over NADPH. The serine dehydrogenase from Pyrococcus horikoshii uses NAD as a coenzyme [24]. The sulfate reduction pathway, which converts sulfate to sulfide, uses two NADPH-utilizing enzymes, PAPS reductase and sulfite reductase. An NADH-dependent sulfite reductase has been identified in Thiobacillus ferrooxidans [29] and Salmonella typhimurium [30]. By altering the cofactor specificity of pathway enzymes to use NADH, the NADPH demand of the pathway is lowered. The yield enhancement from altering the cofactor specificity of these enzymes arises from decreased pentose phosphate flux which produces NADPH but also results in CO₂ loss by 6-phosphogluconate dehydrogenase (gnd) [25]. Several examples are altering the cofactor specificity of enzymes to use NADH preferentially to NADPH are known [26-28]. For enzymes that cannot be altered to utilize NADH, the yield of cystathionine can be further enhanced by altering the pathway specificity of glyceraldehyde 3-phosphate dehydrogenase (GAPDH) to use NADPH preferentially over NADH and providing NADPH to pathway enzymes without the loss of CO₂.

TABLE 4
Host evaluation results for Yarrowia lipolytica strains engineered to produce cystathionine

| | | E1 | Enzyme 1 - | Enzyme 1 - | E1 Codon | E2 | Enzyme 2 - | Enzyme 2 - |
|---|---|---|---|---|---|---|---|---|
| Strain | Titer | Uniprot | activity | source | Optimization | Uniprot | activity | source |
| name | (μg/L) | ID | name | organism | Abbrev. | ID | name | organism |
| YICYTHIO_01 | 0 | N1P5Z1 | Cystathionine | Saccharomyces | Bacillus | R9TW27 | Cystathionine | Bacillus |
| | | | beta- | cerevisiae | subtillus | | gamma- | paralicheniformis |
| | | | synthase | CEN.PK113-7D | | | synthase | ATCC 9945a |
| | | | | | | | Metl | |
| YICYTHIO_02 | 0 | N1P5Z1 | Cystathionine | Saccharomyces | Saccharomyces | R9TW27 | Cystathionine | Bacillus |
| | | | beta- | cerevisiae | cerevisiae | | gamma- | paralicheniformis |
| | | | synthase | CEN.PK113-7D | | | synthase | ATCC 9945a |
| | | | | | | | Metl | |
| YICYTHIO_03 | 92.46 | N1P5Z1 | Cystathionine | Saccharomyces | Yarrowia | R9TW27 | Cystathionine | Bacillus |
| | | | beta- | cerevisiae | lipolytica | | gamma- | paralicheniformis |
| | | | synthase | CEN.PK113-7D | | | synthase | ATCC 9945a |
| | | | | | | | Metl | |
| YICYTHIO_04 | 0 | N1P5Z1 | Cystathionine | Saccharomyces | Bacillus | R9TW27 | Cystathionine | Bacillus |
| | | | beta- | cerevisiae | subtillus | | gamma- | paralicheniformis |
| | | | synthase | CEN.PK113-7D | | | synthase | ATCC 9945a |
| | | | | | | | Metl | |
| YICYTHIO_05 | 0 | N1P5Z1 | Cystathionine | Saccharomyces | Saccharomyces | R9TW27 | Cystathionine | Bacillus |
| | | | beta- | cerevisiae | cerevisiae | | gamma- | paralicheniformis |
| | | | synthase | CEN.PK113-7D | | | synthase | ATCC 9945a |
| | | | | | | | Metl | |
| YICYTHIO_06 | 24.99 | N1P5Z1 | Cystathionine | Saccharomyces | Yarrowia | R9TW27 | Cystathionine | Bacillus |
| | | | beta- | cerevisiae | lipolytica | | gamma- | paralicheniformis |
| | | | synthase | CEN.PK113-7D | | | synthase | ATCC 9945a |
| | | | | | | | Metl | |
| YICYTHIO_07 | 0 | N1P5Z1 | Cystathionine | Saccharomyces | Bacillus | P00935 | Cystathionine | Escherichia |
| | | | beta- | cerevisiae | subtillus | | gamma- | coli |
| | | | synthase | CEN.PK113-7D | | | synthase | (strain K12) |
| YICYTHIO_08 | 0 | N1P5Z1 | Cystathionine | Saccharomyces | Saccharomyces | P00935 | Cystathionine | Escherichia |
| | | | beta- | cerevisiae | cerevisiae | | gamma- | coli |
| | | | synthase | CEN.PK113-7D | | | synthase | (strain K12) |
| YICYTHIO_09 | 75.04 | N1P5Z1 | Cystathionine | Saccharomyces | Yarrowia | P00935 | Cystathionine | Escherichia |
| | | | beta- | cerevisiae | lipolytica | | gamma- | coli |
| | | | synthase | CEN.PK113-7D | | | synthase | (strain K12) |
| YICYTHIO_10 | 0 | N1P5Z1 | Cystathionine | Saccharomyces | Bacillus | P00935 | Cystathionine | Escherichia |
| | | | beta- | cerevisiae | subtillus | | gamma- | coli |
| | | | synthase | CEN.PK113-7D | | | synthase | (strain K12) |
| YICYTHIO_11 | 0 | N1P5Z1 | Cystathionine | Saccharomyces | modified | P00935 | Cystathionine | Escherichia |
| | | | beta- | cerevisiae | codon | | gamma- | coli |
| | | | synthase | CEN.PK113-7D | usage | | synthase | (strain K12) |
| | | | | | for Cg | | | |
| | | | | | and Sc | | | |
| YICYTHIO_12 | 0 | N1P5Z1 | Cystathionine | Saccharomyces | Saccharomyces | P00935 | Cystathionine | Escherichia |
| | | | beta- | cerevisiae | cerevisiae | | gamma- | coli |
| | | | synthase | CEN.PK113-7D | | | synthase | (strain K12) |
| YICYTHIO_13 | 22.68 | N1P5Z1 | Cystathionine | Saccharomyces | Yarrowia | P00935 | Cystathionine | Escherichia |
| | | | beta- | cerevisiae | lipolytica | | gamma- | coli |
| | | | synthase | CEN.PK113-7D | | | synthase | (strain K12) |
| YICYTHIO_14 | 0 | N1P5Z1 | Cystathionine | Saccharomyces | Bacillus | P32801 | Serine/ | Saccharomyces |
| | | | beta- | cerevisiae | subtillus | | threonine- | cerevisiae |
| | | | synthase | CEN.PK113-7D | | | protein | S288c |
| | | | | | | | kinase ELM1 | |
| YICYTHIO_15 | 88.56 | N1P5Z1 | Cystathionine | Saccharomyces | modified | P32801 | Serine/ | Saccharomyces |
| | | | beta- | cerevisiae | codon | | threonine- | cerevisiae |
| | | | synthase | CEN.PK113-7D | usage | | protein | S288c |
| | | | | | for Cg | | kinase ELM1 | |
| | | | | | and Sc | | | |
| YICYTHIO_16 | 0 | N1P5Z1 | Cystathionine | Saccharomyces | Saccharomyces | P32801 | Serine/ | Saccharomyces |
| | | | beta- | cerevisiae | cerevisiae | | threonine- | cerevisiae |
| | | | synthase | CEN.PK113-7D | | | protein | S288c |
| | | | | | | | kinase ELM1 | |
| YICYTHIO_17 | 85.84 | N1P5Z1 | Cystathionine | Saccharomyces | Yarrowia | P32801 | Serine/ | Saccharomyces |
| | | | beta- | cerevisiae | lipolytica | | threonine- | cerevisiae |
| | | | synthase | CEN.PK113-7D | | | protein | S288c |
| | | | | | | | kinase ELM1 | |
| YICYTHIO_18 | 0 | N1P5Z1 | Cystathionine | Saccharomyces | Bacillus | | | |
| | | | beta- | cerevisiae | subtillus | | | |
| | | | synthase | CEN.PK113-7D | | | | |
| YICYTHIO_19 | 0 | N1P5Z1 | Cystathionine | Saccharomyces | Saccharomyces | | | |
| | | | beta- | cerevisiae | cerevisiae | | | |
| | | | synthase | CEN.PK113-7D | | | | |
| YICYTHIO_20 | 6.63 | N1P5Z1 | Cystathionine | Saccharomyces | Yarrowia | | | |
| | | | beta- | cerevisiae | lipolytica | | | |
| | | | synthase | CEN.PK113-7D | | | | |

| | E2 Codon | E3 | Enzyme 3 - | | Enzyme 3 - | E3 Codon |
|---|---|---|---|---|---|---|
| Strain | Optimization | Uniprot | activity | E3 | source | Optimization |
| name | Abbrev. | ID | name | Modifications | organism | Abbrev. |
| YICYTHIO_01 | Bacillus | P10869 | Aspartokinase | G452D | Saccharomyces | Bacillus |
| | subtillus | | | | cerevisiae | subtillus |
| | | | | | S288c | |
| YICYTHIO_02 | Saccharomyces | P10869 | Aspartokinase | G452D | Saccharomyces | Saccharomyces |
| | cerevisiae | | | | cerevisiae | cerevisiae |
| | | | | | S288c | |
| YICYTHIO_03 | Yarrowia | P10869 | Aspartokinase | G452D | Saccharomyces | Yarrowia |
| | lipolytica | | | | cerevisiae | lipolytica |
| | | | | | S288c | |
| YICYTHIO_04 | Bacillus | N1NZ14 | Aspartate | | Saccharomyces | Bacillus |
| | subtillus | | aminotransferase | | cerevisiae | subtillus |
| | | | | | CEN.PK113-7D | |
| YICYTHIO_05 | Saccharomyces | N1NZ14 | Aspartate | | Saccharomyces | Saccharomyces |
| | cerevisiae | | aminotransferase | | cerevisiae | cerevisiae |
| | | | | | CEN.PK113-7D | |
| YICYTHIO_06 | Yarrowia | N1NZ14 | Aspartate | | Saccharomyces | Yarrowia |
| | lipolytica | | aminotransferase | | cerevisiae | lipolytica |
| | | | | | CEN.PK113-7D | |
| YICYTHIO_07 | Bacillus | P10869 | Aspartokinase | G452D | Saccharomyces | Bacillus |
| | subtillus | | | | cerevisiae | subtillus |
| | | | | | S288c | |
| YICYTHIO_08 | Saccharomyces | P10869 | Aspartokinase | G452D | Saccharomyces | Saccharomyces |
| | cerevisiae | | | | cerevisiae | cerevisiae |
| | | | | | S288c | |
| YICYTHIO_09 | Yarrowia | P10869 | Aspartokinase | G452D | Saccharomyces | Yarrowia |
| | lipolytica | | | | cerevisiae | lipolytica |
| | | | | | S288c | |
| YICYTHIO_10 | Bacillus | N1NZ14 | Aspartate | | Saccharomyces | Bacillus |
| | subtillus | | aminotransferase | | cerevisiae | subtillus |
| | | | | | CEN.PK113-7D | |
| YICYTHIO_11 | modified | N1NZ14 | Aspartate | | Saccharomyces | modified |
| | codon | | aminotransferase | | cerevisiae | codon |
| | usage | | | | CEN.PK113-7D | usage |
| | for Cg | | | | | for Cg |
| | and Sc | | | | | and Sc |
| YICYTHIO_12 | Saccharomyces | N1NZ14 | Aspartate | | Saccharomyces | Saccharomyces |
| | cerevisiae | | aminotransferase | | cerevisiae | cerevisiae |
| | | | | | CEN.PK113-7D | |
| YICYTHIO_13 | Yarrowia | N1NZ14 | Aspartate | | Saccharomyces | Yarrowia |
| | lipolytica | | aminotransferase | | cerevisiae | lipolytica |
| | | | | | CEN.PK113-7D | |
| YICYTHIO_14 | Bacillus | P10869 | Aspartokinase | G452D | Saccharomyces | Bacillus |
| | subtillus | | | | cerevisiae | subtillus |
| | | | | | S288c | |
| YICYTHIO_15 | modified | P10869 | Aspartokinase | G452D | Saccharomyces | modified |
| | codon | | | | cerevisiae | codon |
| | usage | | | | S288c | usage |
| | for Cg | | | | | for Cg |
| | and Sc | | | | | and Sc |
| YICYTHIO_16 | Saccharomyces | P10869 | Aspartokinase | G452D | Saccharomyces | Saccharomyces |
| | cerevisiae | | | | cerevisiae | cerevisiae |
| | | | | | S288c | |
| YICYTHIO_17 | Yarrowia | P10869 | Aspartokinase | G452D | Saccharomyces | Yarrowia |
| | lipolytica | | | | cerevisiae | lipolytica |
| | | | | | S288c | |
| YICYTHIO_18 | | | | | | |
| YICYTHIO_19 | | | | | | |
| YICYTHIO_20 | | | | | | |

TABLE 5
Host evaluation results for Bacillus subtillus strains engineered to produce cystathionine

| | | E1 | Enzyme 1 - | Enzyme 1 - | E1 Codon | E2 | Enzyme 2 - | Enzyme 2 - |
|---|---|---|---|---|---|---|---|---|
| Strain | Titer | Uniprot | activity | source | Optimization | Uniprot | activity | source |
| name | (μg/L) | ID | name | organism | Abbrev. | ID | name | organism |
| BsCYTHIO_01 | | N1P5Z1 | Cystathionine | Saccharomyces | Yarrowia | R9TW27 | Cystathionine | Bacillus |
| | | | beta- | cerevisiae | lipolytica | | gamma- | paralicheniformis |
| | | | synthase | CEN.PK113-7D | | | synthase Metl | ATCC 9945a |
| BsCYTHIO_02 | 925.2 | N1P5Z1 | Cystathionine | Saccharomyces | Bacillus | R9TW27 | Cystathionine | Bacillus |
| | | | beta- | cerevisiae | subtillus | | gamma- | paralicheniformis |
| | | | synthase | CEN.PK113-7D | | | synthase Metl | ATCC 9945a |
| BsCYTHIO_03 | | N1P5Z1 | Cystathionine | Saccharomyces | modified | R9TW27 | Cystathionine | Bacillus |
| | | | beta- | cerevisiae | codon | | gamma- | paralicheniformis |
| | | | synthase | CEN.PK113-7D | usage | | synthase Metl | ATCC 9945a |
| | | | | | for Cg | | | |
| | | | | | and Sc | | | |
| BsCYTHIO_04 | — | N1P5Z1 | Cystathionine | Saccharomyces | Saccharomyces | R9TW27 | Cystathionine | Bacillus |
| | | | beta- | cerevisiae | cerevisiae | | gamma- | paralicheniformis |
| | | | synthase | CEN.PK113-7D | | | synthase Metl | ATCC 9945a |
| BsCYTHIO_05 | | N1P5Z1 | Cystathionine | Saccharomyces | Yarrowia | R9TW27 | Cystathionine | Bacillus |
| | | | beta- | cerevisiae | lipolytica | | gamma- | paralicheniformis |
| | | | synthase | CEN.PK113-7D | | | synthase Metl | ATCC 9945a |
| BsCYTHIO_06 | 1084.24 | N1P5Z1 | Cystathionine | Saccharomyces | Bacillus | R9TW27 | Cystathionine | Bacillus |
| | | | beta- | cerevisiae | subtillus | | gamma- | paralicheniformis |
| | | | synthase | CEN.PK113-7D | | | synthase Metl | ATCC 9945a |
| BsCYTHIO_07 | | N1P5Z1 | Cystathionine | Saccharomyces | modified | R9TW27 | Cystathionine | Bacillus |
| | | | beta- | cerevisiae | codon | | gamma- | paralicheniformis |
| | | | synthase | CEN.PK113-7D | usage | | synthase Metl | ATCC 9945a |
| | | | | | for Cg | | | |
| | | | | | and Sc | | | |
| BsCYTHIO_08 | | N1P5Z1 | Cystathionine | Saccharomyces | Saccharomyces | R9TW27 | Cystathionine | Bacillus |
| | | | beta- | cerevisiae | cerevisiae | | gamma- | paralicheniformis |
| | | | synthase | CEN.PK113-7D | | | synthase Metl | ATCC 9945a |
| BsCYTHIO_09 | 448.72 | N1P5Z1 | Cystathionine | Saccharomyces | Bacillus | P00935 | Cystathionine | Escherichia |
| | | | beta- | cerevisiae | subtillus | | gamma- | coli |
| | | | synthase | CEN.PK113-7D | | | synthase | (strain K12) |
| BsCYTHIO_10 | | N1P5Z1 | Cystathionine | Saccharomyces | modified | P00935 | Cystathionine | Escherichia |
| | | | beta- | cerevisiae | codon | | gamma- | coli |
| | | | synthase | CEN.PK113-7D | usage | | synthase | (strain K12) |
| | | | | | for Cg | | | |
| | | | | | and Sc | | | |
| BsCYTHIO_11 | | N1P5Z1 | Cystathionine | Saccharomyces | Saccharomyces | P00935 | Cystathionine | Escherichia |
| | | | beta- | cerevisiae | cerevisiae | | gamma- | coli |
| | | | synthase | CEN.PK113-7D | | | synthase | (strain K12) |
| BsCYTHIO_12 | | N1P5Z1 | Cystathionine | Saccharomyces | Yarrowia | P00935 | Cystathionine | Escherichia |
| | | | beta- | cerevisiae | lipolytica | | gamma- | coli |
| | | | synthase | CEN.PK113-7D | | | synthase | (strain K12) |
| BsCYTHIO_13 | 1016.19 | N1P5Z1 | Cystathionine | Saccharomyces | Bacillus | P00935 | Cystathionine | Escherichia |
| | | | beta- | cerevisiae | subtillus | | gamma- | coli |
| | | | synthase | CEN.PK113-7D | | | synthase | (strain K12) |
| BsCYTHIO_14 | | N1P5Z1 | Cystathionine | Saccharomyces | modified | P00935 | Cystathionine | Escherichia |
| | | | beta- | cerevisiae | codon | | gamma- | coli |
| | | | synthase | CEN.PK113-7D | usage | | synthase | (strain K12) |
| | | | | | for Cg | | | |
| | | | | | and Sc | | | |
| BsCYTHIO_15 | | N1P5Z1 | Cystathionine | Saccharomyces | Saccharomyces | P00935 | Cystathionine | Escherichia |
| | | | beta- | cerevisiae | cerevisiae | | gamma- | coli |
| | | | synthase | CEN.PK113-7D | | | synthase | (strain K12) |
| BsCYTHIO_16 | | N1P5Z1 | Cystathionine | Saccharomyces | Yarrowia | P00935 | Cystathionine | Escherichia |
| | | | beta- | cerevisiae | lipolytica | | gamma- | coli |
| | | | synthase | CEN.PK113-7D | | | synthase | (strain K12) |
| BsCYTHIO_17 | | N1P5Z1 | Cystathionine | Saccharomyces | Bacillus | P32801 | Serine/ | Saccharomyces |
| | | | beta- | cerevisiae | subtillus | | threonine- | cerevisiae |
| | | | synthase | CEN.PK113-7D | | | protein kinase | S288c |
| | | | | | | | ELM1 | |
| BsCYTHIO_18 | | N1P5Z1 | Cystathionine | Saccharomyces | modified | | | |
| | | | beta- | cerevisiae | codon | | | |
| | | | synthase | CEN.PK113-7D | usage | | | |
| | | | | | for Cg | | | |
| | | | | | and Sc | | | |
| BsCYTHIO_19 | | N1P5Z1 | Cystathionine | Saccharomyces | Saccharomyces | P32801 | Serine/ | Saccharomyces |
| | | | beta- | cerevisiae | cerevisiae | | threonine- | cerevisiae |
| | | | synthase | CEN.PK113-7D | | | protein kinase | S288c |
| | | | | | | | ELM1 | |
| BsCYTHIO_20 | | N1P5Z1 | Cystathionine | Saccharomyces | Yarrowia | P32801 | Serine/ | Saccharomyces |
| | | | beta- | cerevisiae | lipolytica | | threonine- | cerevisiae |
| | | | synthase | CEN.PK113-7D | | | protein kinase | S288c |
| | | | | | | | ELM1 | |
| BsCYTHIO_21 | | N1P5Z1 | Cystathionine | Saccharomyces | Bacillus | | | |
| | | | beta- | cerevisiae | subtillus | | | |
| | | | synthase | CEN.PK113-7D | | | | |
| BsCYTHIO_22 | | N1P5Z1 | Cystathionine | Saccharomyces | modified | | | |
| | | | beta- | cerevisiae | codon | | | |
| | | | synthase | CEN.PK113-7D | usage | | | |
| | | | | | for Cg | | | |
| | | | | | and Sc | | | |
| BsCYTHIO_23 | 306.4 | N1P5Z1 | Cystathionine | Saccharomyces | Saccharomyces | | | |
| | | | beta- | cerevisiae | cerevisiae | | | |
| | | | synthase | CEN.PK113-7D | | | | |
| BsCYTHIO_24 | 232.48 | N1P5Z1 | Cystathionine | Saccharomyces | Yarrowia | | | |
| | | | beta- | cerevisiae | lipolytica | | | |
| | | | synthase | CEN.PK113-7D | | | | |
| BsCYTHIO_25 | 0 | N1P5Z1 | Cystathionine | Saccharomyces | | | | |
| | | | beta- | cerevisiae | | | | |
| | | | synthase | CEN.PK113-7D | | | | |

| | E2 Codon | E3 | Enzyme 3 - | | Enzyme 3 - | E3 Codon |
|---|---|---|---|---|---|---|
| Strain | Optimization | Uniprot | activity | E3 | source | Optimization |
| name | Abbrev. | ID | name | Modifications | organism | Abbrev. |
| BsCYTHIO_01 | Yarrowia | N1NZ14 | Aspartate | | Saccharomyces | Yarrowia |
| | lipolytica | | aminotransferase | | cerevisiae | lipolytica |
| | | | | | (strain | |
| | | | | | CEN.PK113-7D) | |
| | | | | | (Baker's | |
| | | | | | yeast) | |
| BsCYTHIO_02 | Bacillus | P10869 | Aspartokinase | G452D | Saccharomyces | Bacillus |
| | subtillus | | | | cerevisiae | subtillus |
| | | | | | (strain | |
| | | | | | ATCC 204508/ | |
| | | | | | S288c) | |
| | | | | | (Baker's | |
| | | | | | yeast) | |
| BsCYTHIO_03 | modified | | | | | |
| | codon | | | | | |
| | usage | | | | | |
| | for Cg | | | | | |
| | and Sc | | | | | |
| BsCYTHIO_04 | Saccharomyces | P10869 | Aspartokinase | G452D | Saccharomyces | Saccharomyces |
| | cerevisiae | | | | cerevisiae | cerevisiae |
| | | | | | (strain | |
| | | | | | ATCC 204508/ | |
| | | | | | S288c) | |
| | | | | | (Baker's | |
| | | | | | yeast) | |
| BsCYTHIO_05 | Yarrowia | P10869 | Aspartokinase | G452D | Saccharomyces | Yarrowia |
| | lipolytica | | | | cerevisiae | lipolytica |
| | | | | | (strain | |
| | | | | | ATCC 204508/ | |
| | | | | | S288c) | |
| | | | | | (Baker's | |
| | | | | | yeast) | |
| BsCYTHIO_06 | Bacillus | N1NZ14 | Aspartate | | Saccharomyces | Bacillus |
| | subtillus | | aminotransferase | | cerevisiae | subtillus |
| | | | | | (strain | |
| | | | | | CEN.PK113-7D) | |
| | | | | | (Baker's | |
| | | | | | yeast) | |
| BsCYTHIO_07 | modified | N1NZ14 | Aspartate | | Saccharomyces | modified |
| | codon | | aminotransferase | | cerevisiae | codon |
| | usage | | | | (strain | usage |
| | for Cg | | | | CEN.PK113-7D) | for Cg |
| | and Sc | | | | (Baker's | and Sc |
| | | | | | yeast) | |
| BsCYTHIO_08 | Saccharomyces | N1NZ14 | Aspartate | | Saccharomyces | Saccharomyces |
| | cerevisiae | | aminotransferase | | cerevisiae | cerevisiae |
| | | | | | (strain | |
| | | | | | CEN.PK113-7D) | |
| | | | | | (Baker's | |
| | | | | | yeast) | |
| BsCYTHIO_09 | Bacillus | P10869 | Aspartokinase | G452D | Saccharomyces | Bacillus |
| | subtillus | | | | cerevisiae | subtillus |
| | | | | | (strain | |
| | | | | | ATCC 204508/ | |
| | | | | | S288c) | |
| | | | | | (Baker's | |
| | | | | | yeast) | |
| BsCYTHIO_10 | modified | | | | | |
| | codon | | | | | |
| | usage | | | | | |
| | for Cg | | | | | |
| | and Sc | | | | | |
| BsCYTHIO_11 | Saccharomyces | P10869 | Aspartokinase | G452D | Saccharomyces | Saccharomyces |
| | cerevisiae | | | | cerevisiae | cerevisiae |
| | | | | | (strain | |
| | | | | | ATCC 204508/ | |
| | | | | | S288c) | |
| | | | | | (Baker's | |
| | | | | | yeast) | |
| BsCYTHIO_12 | Yarrowia | P10869 | Aspartokinase | G452D | Saccharomyces | Yarrowia |
| | lipolytica | | | | cerevisiae | lipolytica |
| | | | | | (strain | |
| | | | | | ATCC 204508/ | |
| | | | | | S288c) | |
| | | | | | (Baker's | |
| | | | | | yeast) | |
| BsCYTHIO_13 | Bacillus | N1NZ14 | Aspartate | | Saccharomyces | Bacillus |
| | subtillus | | aminotransferase | | cerevisiae | subtillus |
| | | | | | (strain | |
| | | | | | CEN.PK113-7D) | |
| | | | | | (Baker's | |
| | | | | | yeast) | |
| BsCYTHIO_14 | modified | N1NZ14 | Aspartate | | Saccharomyces | modified |
| | codon | | aminotransferase | | cerevisiae | codon |
| | usage | | | | (strain | usage |
| | for Cg | | | | CEN.PK113-7D) | for Cg |
| | and Sc | | | | (Baker's | and Sc |
| | | | | | yeast) | |
| BsCYTHIO_15 | Saccharomyces | N1NZ14 | Aspartate | | Saccharomyces | Saccharomyces |
| | cerevisiae | | aminotransferase | | cerevisiae | cerevisiae |
| | | | | | (strain | |
| | | | | | CEN.PK113-7D) | |
| | | | | | (Baker's | |
| | | | | | yeast) | |
| BsCYTHIO_16 | Yarrowia | N1NZ14 | Aspartate | | Saccharomyces | Yarrowia |
| | lipolytica | | aminotransferase | | cerevisiae | lipolytica |
| | | | | | (strain | |
| | | | | | CEN.PK113-7D) | |
| | | | | | (Baker's | |
| | | | | | yeast) | |
| BsCYTHIO_17 | Bacillus | P10869 | Aspartokinase | G452D | Saccharomyces | Bacillus |
| | subtillus | | | | cerevisiae | subtillus |
| | | | | | (strain | |
| | | | | | ATCC 204508/ | |
| | | | | | S288c) | |
| | | | | | (Baker's | |
| | | | | | yeast) | |
| BsCYTHIO_18 | | | | | | |
| BsCYTHIO_19 | Saccharomyces | P10869 | Aspartokinase | G452D | Saccharomyces | Saccharomyces |
| | cerevisiae | | | | cerevisiae | cerevisiae |
| | | | | | (strain | |
| | | | | | ATCC 204508/ | |
| | | | | | S288c) | |
| | | | | | (Baker's | |
| | | | | | yeast) | |
| BsCYTHIO_20 | Yarrowia | P10869 | Aspartokinase | G452D | Saccharomyces | Yarrowia |
| | lipolytica | | | | cerevisiae | lipolytica |
| | | | | | (strain | |
| | | | | | ATCC 204508/ | |
| | | | | | S288c) | |
| | | | | | (Baker's | |
| | | | | | yeast) | |
| BsCYTHIO_21 | | | | | | |
| BsCYTHIO_22 | | | | | | |
| BsCYTHIO_23 | | | | | | |
| BsCYTHIO_24 | | | | | | |
| BsCYTHIO_25 | | | | | | |

TABLE 6
Host evaluation results for Saccharomyces cerevisiae strains engineered to produce cystathionine

| | | E1 | Enzyme 1 - | Enzyme 1 - | E1 Codon | E2 | Enzyme 2 - | Enzyme 2 - |
|---|---|---|---|---|---|---|---|---|
| Strain | Titer | Uniprot | activity | source | Optimization | Uniprot | activity | source |
| name | (μg/L) | ID | name | organism | Abbrev. | ID | name | organism |
| ScCYTHIO_131 | 21.85 | N1P5Z1 | Cystathionine | Saccharomyces | modified | R9TW27 | Cystathionine | Bacillus |
| | | | beta- | cerevisiae | codon | | gamma- | paralicheniformis |
| | | | synthase | CEN.PK113-7D | usage | | synthase | ATCC 9945a |
| | | | | | for Cg | | Metl | |
| | | | | | and Sc | | | |
| ScCYTHIO_132 | | N1P5Z1 | Cystathionine | Saccharomyces | Saccharomyces | R9TW27 | Cystathionine | Bacillus |
| | | | beta- | cerevisiae | cerevisiae | | gamma- | paralicheniformis |
| | | | synthase | CEN.PK113-7D | | | synthase | ATCC 9945a |
| | | | | | | | Metl | |
| ScCYTHIO_133 | 105.24 | N1P5Z1 | Cystathionine | Saccharomyces | Yarrowia | R9TW27 | Cystathionine | Bacillus |
| | | | beta- | cerevisiae | lipolytica | | gamma- | paralicheniformis |
| | | | synthase | CEN.PK113-7D | | | synthase | ATCC 9945a |
| | | | | | | | Metl | |
| ScCYTHIO_134 | 360.226 | N1P5Z1 | Cystathionine | Saccharomyces | Saccharomyces | P00935 | Cystathionine | Escherichia |
| | | | beta- | cerevisiae | cerevisiae | | gamma- | coli |
| | | | synthase | CEN.PK113-7D | | | synthase | (strain K12) |
| ScCYTHIO_135 | | N1P5Z1 | Cystathionine | Saccharomyces | Bacillus | P00935 | Cystathionine | Escherichia |
| | | | beta- | cerevisiae | subtillus | | gamma- | coli |
| | | | synthase | CEN.PK113-7D | | | synthase | (strain K12) |
| ScCYTHIO_136 | 25.30 | N1P5Z1 | Cystathionine | Saccharomyces | Saccharomyces | P00935 | Cystathionine | Escherichia |
| | | | beta- | cerevisiae | cerevisiae | | gamma- | coli |
| | | | synthase | CEN.PK113-7D | | | synthase | (strain K12) |
| ScCYTHIO_137 | 31.56 | N1P5Z1 | Cystathionine | Saccharomyces | Yarrowia | P00935 | Cystathionine | Escherichia |
| | | | beta- | cerevisiae | lipolytica | | gamma- | coli |
| | | | synthase | CEN.PK113-7D | | | synthase | (strain K12) |
| ScCYTHIO_138 | 42.19 | N1P5Z1 | Cystathionine | Saccharomyces | Saccharomyces | P32801 | Serine/ | Saccharomyces |
| | | | beta- | cerevisiae | cerevisiae | | threonine- | cerevisiae |
| | | | synthase | CEN.PK113-7D | | | protein | S288c |
| | | | | | | | kinase ELM1 | |
| ScCYTHIO_139 | 8.79 | N1P5Z1 | Cystathionine | Saccharomyces | Bacillus | | | |
| | | | beta- | cerevisiae | subtillus | | | |
| | | | synthase | CEN.PK113-7D | | | | |
| ScCYTHIO_140 | 6.00 | N1P5Z1 | Cystathionine | Saccharomyces | Saccharomyces | | | |
| | | | beta- | cerevisiae | cerevisiae | | | |
| | | | synthase | CEN.PK113-7D | | | | |
| ScCYTHIO_141 | 2.58 | N1P5Z1 | Cystathionine | Saccharomyces | Yarrowia | | | |
| | | | beta- | cerevisiae | lipolytica | | | |
| | | | synthase | CEN.PK113-7D | | | | |

| | E2 Codon | E3 | Enzyme 3 - | | Enzyme 3 - | E3 Codon |
|---|---|---|---|---|---|---|
| Strain | Optimization | Uniprot | activity | E3 | source | Optimization |
| name | Abbrev. | ID | name | Modifications | organism | Abbrev. |
| ScCYTHIO_131 | modified | N1NZ14 | Aspartate | | Saccharomyces | modified |
| | codon | | aminotransferase | | cerevisiae | codon |
| | usage | | | | CEN.PK113-7D | usage |
| | for Cg | | | | | for Cg |
| | and Sc | | | | | and Sc |
| ScCYTHIO_132 | Saccharomyces | N1NZ14 | Aspartate | | Saccharomyces | Saccharomyces |
| | cerevisiae | | aminotransferase | | cerevisiae | cerevisiae |
| | | | | | CEN.PK113-7D | |
| ScCYTHIO_133 | Yarrowia | N1NZ14 | Aspartate | | Saccharomyces | Yarrowia |
| | lipolytica | | aminotransferase | | cerevisiae | lipolytica |
| | | | | | CEN.PK113-7D | |
| ScCYTHIO_134 | Saccharomyces | P10869 | Aspartokinase | G452D | Saccharomyces | Saccharomyces |
| | cerevisiae | | | | cerevisiae | cerevisiae |
| | | | | | S288c | |
| ScCYTHIO_135 | Bacillus | N1NZ14 | Aspartate | | Saccharomyces | Bacillus |
| | subtillus | | aminotransferase | | cerevisiae | subtillus |
| | | | | | CEN.PK113-7D | |
| ScCYTHIO_136 | Saccharomyces | N1NZ14 | Aspartate | | Saccharomyces | Saccharomyces |
| | cerevisiae | | aminotransferase | | cerevisiae | cerevisiae |
| | | | | | CEN.PK113-7D | |
| ScCYTHIO_137 | Yarrowia | N1NZ14 | Aspartate | | Saccharomyces | Yarrowia |
| | lipolytica | | aminotransferase | | cerevisiae | lipolytica |
| | | | | | CEN.PK113-7D | |
| ScCYTHIO_138 | Saccharomyces | P10869 | Aspartokinase | G452D | Saccharomyces | Saccharomyces |
| | cerevisiae | | | | cerevisiae | cerevisiae |
| | | | | | S288c | |
| ScCYTHIO_139 | | | | | | |
| ScCYTHIO_140 | | | | | | |
| ScCYTHIO_141 | | | | | | |

TABLE 7
Host evaluation results for Corynebacteria glutamicum strains engineered to produce cystathionine

| | | E1 | Enzyme 1 - | Enzyme 1 - | E1 Codon | E2 | Enzyme 2 - | Enzyme 2 - |
|---|---|---|---|---|---|---|---|---|
| Strain | Titer | Uniprot | activity | source | Optimization | Uniprot | activity | source |
| name | (μg/L) | ID | name | organism | Abbrev. | ID | name | organism |
| CgCYTHIO_12 | | N1P5Z1 | Cystathionine | Saccharomyces | Saccharomyces | R9TW27 | Cystathionine | Bacillus |
| | | | beta- | cerevisiae | cerevisiae | | gamma- | paralicheniformis |
| | | | synthase | CEN.PK113-7D | | | synthase Metl | ATCC 9945a |
| CgCYTHIO_13 | 2141.28 | N1P5Z1 | Cystathionine | Saccharomyces | Yarrowia | R9TW27 | Cystathionine | Bacillus |
| | | | beta- | cerevisiae | lipolytica | | gamma- | paralicheniformis |
| | | | synthase | CEN.PK113-7D | | | synthase Metl | ATCC 9945a |
| CgCYTHIO_14 | | N1P5Z1 | Cystathionine | Saccharomyces | Bacillus | R9TW27 | Cystathionine | Bacillus |
| | | | beta- | cerevisiae | subtillus | | gamma- | paralicheniformis |
| | | | synthase | CEN.PK113-7D | | | synthase Metl | ATCC 9945a |
| CgCYTHIO_15 | 1816.69 | N1P5Z1 | Cystathionine | Saccharomyces | Yarrowia | R9TW27 | Cystathionine | Bacillus |
| | | | beta- | cerevisiae | lipolytica | | gamma- | paralicheniformis |
| | | | synthase | CEN.PK113-7D | | | synthase Metl | ATCC 9945a |
| CgCYTHIO_16 | | N1P5Z1 | Cystathionine | Saccharomyces | Saccharomyces | P00935 | Cystathionine | Escherichia |
| | | | beta- | cerevisiae | cerevisiae | | gamma- | coli |
| | | | synthase | CEN.PK113-7D | | | synthase | (strain K12) |
| CgCYTHIO_17 | 2407.79 | N1P5Z1 | Cystathionine | Saccharomyces | Yarrowia | P00935 | Cystathionine | Escherichia |
| | | | beta- | cerevisiae | lipolytica | | gamma- | coli |
| | | | synthase | CEN.PK113-7D | | | synthase | (strain K12) |
| CgCYTHIO_18 | | N1P5Z1 | Cystathionine | Saccharomyces | Bacillus | P00935 | Cystathionine | Escherichia |
| | | | beta- | cerevisiae | subtillus | | gamma- | coli |
| | | | synthase | CEN.PK113-7D | | | synthase | (strain K12) |
| CgCYTHIO_19 | 3957.83 | N1P5Z1 | Cystathionine | Saccharomyces | modified | P00935 | Cystathionine | Escherichia |
| | | | beta- | cerevisiae | codon | | gamma- | coli |
| | | | synthase | CEN.PK113-7D | usage | | synthase | (strain K12) |
| | | | | | for Cg | | | |
| | | | | | and Sc | | | |
| CgCYTHIO_20 | | N1P5Z1 | Cystathionine | Saccharomyces | Yarrowia | P00935 | Cystathionine | Escherichia |
| | | | beta- | cerevisiae | lipolytica | | gamma- | coli |
| | | | synthase | CEN.PK113-7D | | | synthase | (strain K12) |
| CgCYTHIO_21 | | N1P5Z1 | Cystathionine | Saccharomyces | modified | P32801 | Serine/threonine- | Saccharomyces |
| | | | beta- | cerevisiae | codon | | protein | cerevisiae |
| | | | synthase | CEN.PK113-7D | usage | | kinase ELM1 | S288c |
| | | | | | for Cg | | | |
| | | | | | and Sc | | | |
| CgCYTHIO_22 | | N1P5Z1 | Cystathionine | Saccharomyces | Saccharomyces | P32801 | Serine/threonine- | Saccharomyces |
| | | | beta- | cerevisiae | cerevisiae | | protein | cerevisiae |
| | | | synthase | CEN.PK113-7D | | | kinase ELM1 | S288c |
| CgCYTHIO_23 | | N1P5Z1 | Cystathionine | Saccharomyces | Yarrowia | P32801 | Serine/threonine- | Saccharomyces |
| | | | beta- | cerevisiae | lipolytica | | protein | cerevisiae |
| | | | synthase | CEN.PK113-7D | | | kinase ELM1 | S288c |
| CgCYTHIO_24 | 1937.47 | N1P5Z1 | Cystathionine | Saccharomyces | Bacillus | | | |
| | | | beta- | cerevisiae | subtillus | | | |
| | | | synthase | CEN.PK113-7D | | | | |
| CgCYTHIO_25 | 2763.95 | N1P5Z1 | Cystathionine | Saccharomyces | modified | | | |
| | | | beta- | cerevisiae | codon | | | |
| | | | synthase | CEN.PK113-7D | usage | | | |
| | | | | | for Cg | | | |
| | | | | | and Sc | | | |
| CgCYTHIO_26 | 1739.41 | N1P5Z1 | Cystathionine | Saccharomyces | Yarrowia | | | |
| | | | beta- | cerevisiae | lipolytica | | | |
| | | | synthase | CEN.PK113-7D | | | | |

| | E2 Codon | E3 | Enzyme 3 - | | Enzyme 3 - | E3 Codon |
|---|---|---|---|---|---|---|
| Strain | Optimization | Uniprot | activity | E3 | source | Optimization |
| name | Abbrev. | ID | name | Modifications | organism | Abbrev. |
| CgCYTHIO_12 | Saccharomyces | P10869 | Aspartokinase | G452D | Saccharomyces | Saccharomyces |
| | cerevisiae | | | | cerevisiae | cerevisiae |
| | | | | | (strain | |
| | | | | | ATCC 204508/ | |
| | | | | | S288c) | |
| | | | | | (Baker's | |
| | | | | | yeast) | |
| CgCYTHIO_13 | Yarrowia | P10869 | Aspartokinase | G452D | Saccharomyces | Yarrowia |
| | lipolytica | | | | cerevisiae | lipolytica |
| | | | | | (strain | |
| | | | | | ATCC 204508/ | |
| | | | | | S288c) | |
| | | | | | (Baker's | |
| | | | | | yeast) | |
| CgCYTHIO_14 | Bacillus | N1NZ14 | Aspartate | | Saccharomyces | Bacillus |
| | subtillus | | aminotransferase | | cerevisiae | subtillus |
| | | | | | (strain | |
| | | | | | CEN.PK113-7D) | |
| | | | | | (Baker's | |
| | | | | | yeast) | |
| CgCYTHIO_15 | Yarrowia | N1NZ14 | Aspartate | | Saccharomyces | Yarrowia |
| | lipolytica | | aminotransferase | | cerevisiae | lipolytica |
| | | | | | (strain | |
| | | | | | CEN.PK113-7D) | |
| | | | | | (Baker's | |
| | | | | | yeast) | |
| CgCYTHIO_16 | Saccharomyces | P10869 | Aspartokinase | G452D | Saccharomyces | Saccharomyces |
| | cerevisiae | | | | cerevisiae | cerevisiae |
| | | | | | (strain | |
| | | | | | ATCC 204508/ | |
| | | | | | S288c) | |
| | | | | | (Baker's | |
| | | | | | yeast) | |
| CgCYTHIO_17 | Yarrowia | P10869 | Aspartokinase | G452D | Saccharomyces | Yarrowia |
| | lipolytica | | | | cerevisiae | lipolytica |
| | | | | | (strain | |
| | | | | | ATCC 204508/ | |
| | | | | | S288c) | |
| | | | | | (Baker's | |
| | | | | | yeast) | |
| CgCYTHIO_18 | Bacillus | N1NZ14 | Aspartate | | Saccharomyces | Bacillus |
| | subtillus | | aminotransferase | | cerevisiae | subtillus |
| | | | | | (strain | |
| | | | | | CEN.PK113-7D) | |
| | | | | | (Baker's | |
| | | | | | yeast) | |
| CgCYTHIO_19 | modified | N1NZ14 | Aspartate | | Saccharomyces | modified |
| | codon | | aminotransferase | | cerevisiae | codon |
| | usage | | | | (strain | usage for |
| | for Cg | | | | CEN.PK113-7D) | Corynebacterium |
| | and Sc | | | | (Baker's | glutamicum |
| | | | | | yeast) | and |
| | | | | | | Saccharomyces |
| | | | | | | cerevisiae |
| CgCYTHIO_20 | Yarrowia | N1NZ14 | Aspartate | | Saccharomyces | Yarrowia |
| | lipolytica | | aminotransferase | | cerevisiae | lipolytica |
| | | | | | (strain | |
| | | | | | CEN.PK113-7D) | |
| | | | | | (Baker's | |
| | | | | | yeast) | |
| CgCYTHIO_21 | | UniProt_ID | published_enzyme_name | modifications | Saccharomyces | modified |
| | | | | | cerevisiae | codon |
| | | | | | (strain | usage |
| | | | | | ATCC 204508/ | for Cg |
| | | | | | S288c) | and Sc |
| | | | | | (Baker's | |
| | | | | | yeast) | |
| CgCYTHIO_22 | Saccharomyces | P10869 | Aspartokinase | G452D | Saccharomyces | Saccharomyces |
| | cerevisiae | | | | cerevisiae | cerevisiae |
| | | | | | (strain | |
| | | | | | ATCC 204508/ | |
| | | | | | S288c) | |
| | | | | | (Baker's | |
| | | | | | yeast) | |
| CgCYTHIO_23 | Yarrowia | P10869 | Aspartokinase | G452D | Saccharomyces | Yarrowia |
| | lipolytica | | | | cerevisiae | lipolytica |
| | | | | | (strain | |
| | | | | | ATCC 204508/ | |
| | | | | | S288c) | |
| | | | | | (Baker's | |
| | | | | | yeast) | |
| CgCYTHIO_24 | | | | | | |
| CgCYTHIO_25 | | | | | | |
| CgCYTHIO_26 | | | | | | |

TABLE 8
Fourth-round results for Saccharomyces cerevisiae strains engineered to produce cystathionine

| | | E1 | Enzyme 1 - | | Enzyme 1 - | E2 | Enzyme 2 - | |
|---|---|---|---|---|---|---|---|---|
| Strain | Titer | Uniprot | activity | E1 | source | Uniprot | activity | E2 |
| name | (μg/L) | ID | name | Modifications | organism | ID | name | Modifications |
| ScCYTHIO_12 | 19.07 | N1P5Z1 | cystathionine | Error | Saccharomyces | | | |
| | | | beta synthase | | cerevisiae | | | |
| | | | | | S288c | | | |
| ScCYTHIO_92 | | P26512 | Aspartokinase | Q298G | Corynebacterium | | | |
| | | | | | glutamicum | | | |
| | | | | | ATCC 13032 | | | |
| ScCYTHIO_93 | | P00561 | Bifunctional | S345F | Escherichia | | | |
| | | | aspartokinase/ | | coli | | | |
| | | | homoserine | | (strain K12) | | | |
| | | | dehydrogenase | | | | | |
| ScCYTHIO_94 | | P08499 | Homoserine | G378E | Corynebacterium | | | |
| | | | dihydrogenase | | glutamicum | | | |
| | | | | | ATCC 13032 | | | |
| ScCYTHIO_95 | | P08499 | Homoserine | A429L, K430S, | Corynebacterium | | | |
| | | | dihydrogenase | P431L, V432L, | glutamicum | | | |
| | | | | V433L, K434R, | ATCC 13032 | | | |
| | | | | A435Q, I436S, | | | | |
| | | | | N437T, S438V, | | | | |
| | | | | delete AA | | | | |
| | | | | 439-445 | | | | |
| ScCYTHIO_96 | | H7C7K2 | Pyruvate | P458S | Corynebacterium | | | |
| | | | carboxylase | | glutamicum | | | |
| | | | | | ATCC 13032 | | | |
| ScCYTHIO_97 | | P32327 | Pyruvate | | Saccharomyces | | | |
| | | | carboxylase | | cerevisiae | | | |
| | | | | | S288c | | | |
| ScCYTHIO_98 | | P00864 | Phosphoenol | | Escherichia | | | |
| | | | pyruvate | | coli | | | |
| | | | carboxylase | | (strain K12) | | | |
| ScCYTHIO_99 | | P23542 | aspartate | | Saccharomyces | | | |
| | | | aminotransferase | | cerevisiae | | | |
| | | | activity | | S288c | | | |
| ScCYTHIO_100 | 0 | P00509 | aspartate | | Escherichia | | | |
| | | | aminotransferase | | coli | | | |
| | | | activity | | (strain K12) | | | |
| ScCYTHIO_101 | 0 | P13663 | Aspartate- | | Saccharomyces | | | |
| | | | semialdehyde | | cerevisiae | | | |
| | | | dihydrogenase | | S288c | | | |
| ScCYTHIO_102 | 12.26 | H7C7K2 | Pyruvate | P458S | Corynebacterium | P00509 | aspartate | |
| | | | carboxylase | | glutamicum | | aminotransferase | |
| | | | | | ATCC 13032 | | activity | |
| ScCYTHIO_103 | 55.17 | P00864 | Phosphoenol | | Escherichia | P00509 | aspartate | |
| | | | pyruvate | | coli | | aminotransferase | |
| | | | carboxylase | | (strain K12) | | activity | |
| ScCYTHIO_104 | 66.11 | P00864 | Phosphoenol | | Escherichia | P23542 | aspartate | |
| | | | pyruvate | | coli | | aminotransferase | |
| | | | carboxylase | | (strain K12) | | activity | |
| ScCYTHIO_105 | 0 | P40054 | D-3- | | Saccharomyces | | | |
| | | | phosphoglycerate | | cerevisiae | | | |
| | | | dihydrogenase | | S288c | | | |
| ScCYTHIO_106 | 5.65 | P40510 | D-3- | | Saccharomyces | | | |
| | | | phosphoglycerate | | cerevisiae | | | |
| | | | dihydrogenase | | S288c | | | |
| ScCYTHIO_107 | 0 | P33330 | Phosphoserine | | Saccharomyces | | | |
| | | | transaminase | | cerevisiae | | | |
| | | | | | S288c | | | |
| ScCYTHIO_108 | 0 | P42941 | Phosphoserine | | Saccharomyces | | | |
| | | | phosphatase | | cerevisiae | | | |
| | | | | | S288c | | | |
| ScCYTHIO_109 | 0 | P40054 | D-3- | | Saccharomyces | P33330 | Phosphoserine | |
| | | | phosphoglycerate | | cerevisiae | | transaminase | |
| | | | dihydrogenase | | S288c | | | |
| ScCYTHIO_110 | | P33330 | Phosphoserine | | Saccharomyces | P42941 | Phosphoserine | |
| | | | transaminase | | cerevisiae | | phosphatase | |
| | | | | | S288c | | | |
| ScCYTHIO_111 | 0 | P08465 | Homoserine | | Saccharomyces | | | |
| | | | O-acetyltransferase | | cerevisiae | | | |
| | | | | | S288c | | | |
| ScCYTHIO_112 | 0 | P0A9D4 | Serine | M256I | Escherichia | | | |
| | | | acetyltransferase | | coli | | | |
| | | | | | (strain K12) | | | |
| ScCYTHIO_113 | 0 | P0A9D4 | Serine | M256W | Escherichia | | | |
| | | | acetyltransferase | | coli | | | |
| | | | | | (strain K12) | | | |
| ScCYTHIO_114 | 11.40 | P0A9D4 | Serine | M256A | Escherichia | | | |
| | | | acetyltransferase | | coli | | | |
| | | | | | (strain K12) | | | |
| ScCYTHIO_115 | 0 | P0A9D4 | Serine | S253L | Escherichia | | | |
| | | | acetyltransferase | | coli | | | |
| | | | | | (strain K12) | | | |
| ScCYTHIO_116 | | D2Z028 | L-serine/— | | Streptomyces | D9V2L8 | Cysteine | |
| | | | homoserine | | lavendulae | | synthase | |
| | | | O-acetyltransferase | | | | | |
| ScCYTHIO_117 | 10.04 | P08499 | Homoserine | G378E | Corynebacterium | P00561 | Bifunctional | S345F |
| | | | dihydrogenase | | glutamicum | | aspartokinase/ | |
| | | | | | ATCC 13032 | | homoserine | |
| | | | | | | | dihydrogenase | |
| ScCYTHIO_118 | 2.50 | P40573 | Transcriptional | | Saccharomyces | | | |
| | | | activator of | | cerevisiae | | | |
| | | | sulfur | | S288c | | | |
| | | | metabolism | | | | | |
| | | | MET28 | | | | | |
| ScCYTHIO_119 | 0 | P06106 | Homocysteine/ | | Saccharomyces | | | |
| | | | cysteine | | cerevisiae | | | |
| | | | synthase | | S288c | | | |
| ScCYTHIO_120 | 4.93 | P32582 | Cysteine | | Saccharomyces | | | |
| | | | synthase | | cerevisiae | | | |
| | | | | | S288c | | | |
| ScCYTHIO_121 | 21.34 | P08465 | Homoserine | | Saccharomyces | P06106 | Homocysteine/ | |
| | | | O-acetyltransferase | | cerevisiae | | cysteine | |
| | | | | | S288c | | synthase | |
| ScCYTHIO_122 | 0 | P18408 | Phosphoadenosine | | Saccharomyces | | | |
| | | | phosphosulfate | | cerevisiae | | | |
| | | | reductase | | S288c | | | |
| ScCYTHIO_123 | 0 | P08536 | Sulfate | | Saccharomyces | | | |
| | | | adenylyltransferase | | cerevisiae | | | |
| | | | | | S288c | | | |
| ScCYTHIO_124 | 2.19 | Q02196 | Adenylyl- | | Saccharomyces | | | |
| | | | sulfate kinase | | cerevisiae | | | |
| | | | | | S288c | | | |
| ScCYTHIO_125 | 10.08 | P08536 | Sulfate | | Saccharomyces | Q02196 | Adenylyl- | |
| | | | adenylyltransferase | | cerevisiae | | sulfate kinase | |
| | | | | | S288c | | | |
| ScCYTHIO_126 | 11.40 | P08536 | Sulfate | | Saccharomyces | Q02196 | Adenylyl- | |
| | | | adenylyltransferase | | cerevisiae | | sulfate kinase | |
| | | | | | S288c | | | |
| ScCYTHIO_127 | 29.72 | P47169 | Sulfite | | Saccharomyces | P39692 | Sulfite | |
| | | | reductase | | cerevisiae | | reductase | |
| | | | | | S288c | | | |
| ScCYTHIO_128 | 0 | P18408 | Phosphoadenosine | | Saccharomyces | P47169 | Sulfite | |
| | | | phosphosulfate | | cerevisiae | | reductase | |
| | | | reductase | | S288c | | | |
| ScCYTHIO_129 | 2.27 | Q02196 | Adenylyl- | | Saccharomyces | P47169 | Sulfite | |
| | | | sulfate kinase | | cerevisiae | | reductase | |
| | | | | | S288c | | | |

| | Enzyme 2 - | E3 | Enzyme 3 - | | Enzyme 3 - |
|---|---|---|---|---|---|
| Strain | source | Uniprot | activity | E3 | source |
| name | organism | ID | name | Modifications | organism |
| ScCYTHIO_12 | | | | | |
| ScCYTHIO_92 | | | | | |
| ScCYTHIO_93 | | | | | |
| ScCYTHIO_94 | | | | | |
| ScCYTHIO_95 | | | | | |
| ScCYTHIO_96 | | | | | |
| ScCYTHIO_97 | | | | | |
| ScCYTHIO_98 | | | | | |
| ScCYTHIO_99 | | | | | |
| ScCYTHIO_100 | | | | | |
| ScCYTHIO_101 | | | | | |
| ScCYTHIO_102 | Escherichia | P00561 | Bifunctional | S345F | Escherichia |
| | coli | | aspartokinase/ | | coli |
| | (strain K12)* | | homoserine | | (strain K12)* |
| | | | dehydrogenase | | |
| ScCYTHIO_103 | Escherichia | P00561 | Bifunctional | S345F | Escherichia |
| | coli | | aspartokinase/ | | coli |
| | (strain K12)* | | homoserine | | (strain K12)* |
| | | | dehydrogenase | | |
| ScCYTHIO_104 | Saccharomyces | P00561 | Bifunctional | S345F | Escherichia |
| | cerevisiae | | aspartokinase/ | | coli |
| | S288c* | | homoserine | | (strain K12)* |
| | | | dehydrogenase | | |
| ScCYTHIO_105 | | | | | |
| ScCYTHIO_106 | | | | | |
| ScCYTHIO_107 | | | | | |
| ScCYTHIO_108 | | | | | |
| ScCYTHIO_109 | Saccharomyces | P42941 | Phosphoserine | | Saccharomyces |
| | cerevisiae | | phosphatase | | cerevisiae |
| | S288c* | | | | S288c* |
| ScCYTHIO_110 | Saccharomyces | P40054 | D-3- | | Saccharomyces |
| | cerevisiae | | phosphoglycerate | | cerevisiae |
| | S288c* | | dehydrogenase | | S288c* |
| ScCYTHIO_111 | | | | | |
| ScCYTHIO_112 | | | | | |
| ScCYTHIO_113 | | | | | |
| ScCYTHIO_114 | | | | | |
| ScCYTHIO_115 | | | | | |
| ScCYTHIO_116 | Streptomyces | P47164 | cystathionine | | Saccharomyces |
| | sp. AA4* | | gamma-synthase | | cerevisiae |
| | | | | | S288c* |
| ScCYTHIO_117 | Escherichia | H7C7K2 | Pyruvate | P458S | Corynebacterium |
| | coli | | carboxylase | | glutamicum |
| | (strain K12)* | | | | ATCC 13032* |
| ScCYTHIO_118 | | | | | |
| ScCYTHIO_119 | | | | | |
| ScCYTHIO_120 | | | | | |
| ScCYTHIO_121 | Saccharomyces | P32582 | Cysteine synthase | | Saccharomyces |
| | cerevisiae | | | | cerevisiae |
| | S288c* | | | | S288c* |
| ScCYTHIO_122 | | | | | |
| ScCYTHIO_123 | | | | | |
| ScCYTHIO_124 | | | | | |
| ScCYTHIO_125 | Saccharomyces | | | | |
| | cerevisiae | | | | |
| | S288c* | | | | |
| ScCYTHIO_126 | Saccharomyces | P18408 | Phosphoadenosine | | Saccharomyces |
| | cerevisiae | | phosphosulfate | | cerevisiae |
| | S288c* | | reductase | | S288c* |
| ScCYTHIO_127 | Saccharomyces | P06106 | Homocysteine/ | | Saccharomyces |
| | cerevisiae | | cysteine synthase | | cerevisiae |
| | S288c* | | | | S288c* |
| ScCYTHIO_128 | Saccharomyces | P39692 | Sulfite reductase | | Saccharomyces |
| | cerevisiae | | | | cerevisiae |
| | S288c* | | | | S288c* |
| ScCYTHIO_129 | Saccharomyces | P39692 | Sulfite reductase | | Saccharomyces |
| | cerevisiae | | | | cerevisiae |
| | S288c* | | | | S288c* |

E1 Codon Optimization Abbrev.: all strains had modified codon usage for Cg and Sc, except for ScCYTHIO_12, which had native codon usage
E2 and E3 Codon Optimization Abbrev.:
*modified codon usage for Cg and Sc

### REFERENCES

1. Kanehisa, M. and S. Goto, KEGG: kyoto encyclopedia of genes and genomes. Nucleic Acids Res, 2000. 28(1): p. 27-30.

2. Ono, B. I., et al., Cysteine biosynthesis in Saccharomyces cerevisiae: a new outlook on pathway and regulation. Yeast, 1999. 15(13): p. 1365-75.

3. Hebert, A., et al., New insights into sulfur metabolism in yeasts as revealed by studies of Yarrowia lipolytica. Appl Environ Microbiol, 2013. 79(4): p. 1200-11.

4. Sagong, H. Y. and K. J. Kim, Structural Insights into Substrate

Specificity of Cystathionine gamma-Synthase from Corynebacterium glutamicum. J Agric Food Chem, 2017. 65(29): p. 6002-6008.

5. Hwang, B. J., et al., Biochemical analysis on the parallel pathways of methionine biosynthesis in Corynebacterium glutamicum. J Microbiol Biotechnol, 2007. 17(6): p. 1010-7.

6. Lee, H. S. and B. J. Hwang, Methionine biosynthesis and its regulation in Corynebacterium glutamicum: parallel pathways of transsulfuration and direct sulfhydrylation. Appl Microbiol Biotechnol, 2003. 62(5-6): p. 459-67.

7. Hwang, B. J., et al., Corynebacterium glutamicum utilizes both transsulfuration and direct sulfhydrylation pathways for methionine biosynthesis. J Bacteriol, 2002. 184(5): p. 1277-86.

8. Ruckert, C., A. Puhler, and J. Kalinowski, Genome-wide analysis of the L-methionine biosynthetic pathway in Corynebacterium glutamicum by targeted gene deletion and homologous complementation. J Biotechnol, 2003. 104(1-3): p. 213-28.

9. Hullo, M. F., et al., Conversion of methionine to cysteine in Bacillus subtilis and its regulation. J Bacteriol, 2007. 189(1): p. 187-97.

10. Ono, B., et al., Cystathionine accumulation in Saccharomyces cerevisiae. J Bacteriol, 1984. 158(3): p. 860-5.

11. Thomas, D. and Y. Surdin-Kerjan, Metabolism of sulfur amino acids in Saccharomyces cerevisiae. Microbiol Mol Biol Rev, 1997. 61(4): p. 503-32.

12. Nakatani, T., et al, Enhancement of thioredoxin/glutaredoxin-mediated L-cysteine synthesis from S-sulfocysteine increases L-cysteine production in Escherichia coli. Microb Cell Fact, 2012. 11: p. 62.

13. Kikuchi, Y., H. Kojima, and T. Tanaka, Mutational analysis of the feedback sites of lysine-sensitive aspartokinase of Escherichia coli. FEMS Microbiol Lett, 1999. 173(1): p. 211-5.

14. Chen, Z., et al., Integrating molecular dynamics and co-evolutionary analysis for reliable target prediction and deregulation of the allosteric inhibition of aspartokinase for amino acid production. J Biotechnol, 2011. 154(4): p. 248-54.

15. Park, S. D., et al., Characteristics of methionine production by an engineered Corynebacterium glutamicum strain. Metab Eng, 2007. 9(4): p. 327-36.

16. Archer, J. A., D. E. Solow-Cordero, and A. J. Sinskey, A C-terminal deletion in Corynebacterium glutamicum homoserine dehydrogenase abolishes allosteric inhibition by L-threonine. Gene, 1991. 107(1): p. 53-9.

17. Nakamori, S., et al., Overproduction of L-cysteine and L-cystine by Escherichia coli strains with a genetically altered serine acetyltransferase. Appl Environ Microbiol, 1998. 64(5): p. 1607-11.

18. Usuda, Y. and O. Kurahashi, Effects of deregulation of methionine biosynthesis on methionine excretion in Escherichia coli. Appl Environ Microbiol, 2005. 71(6): p. 3228-34.

19. Mordukhova, E. A., D. Kim, and J. G. Pan, Stabilized homoserine o-succinyltransferases (MetA) or L-methionine partially recovers the growth defect in Escherichia coli lacking ATP-dependent proteases or the DnaK chaperone. BMC Microbiol, 2013. 13: p. 179.

20. Takagi, H. and I. Ohtsu, L-Cysteine Metabolism and Fermentation in Microorganisms. Adv Biochem Eng Biotechnol, 2017. 159: p. 129-151.

21. Tenreiro, S., et al., AQR1 gene (ORF YNL065w) encodes a plasma membrane transporter of the major facilitator superfamily that confers resistance to short-chain monocarboxylic acids and quinidine in Saccharomyces cerevisiae. Biochem Biophys Res Commun, 2002. 292(3): p. 741-8.

22. Velasco, I., et al., Saccharomyces cerevisiae Agr1 is an internal-membrane transporter involved in excretion of amino acids. Eukaryot Cell, 2004. 3(6): p. 1492-503.

23. Wu et al. Efficient mining of natural NADH-utilizing dehydrogenases enables systematic cofactor engineering of lysine synthesis pathway of Corynebacterium glutamicum. Metabolic Engineering (2019) 52:77-86.

24. Hayashi et al. Crystal Structures of a Hyperthermophilic Archaeal Homoserine Dehydrogenase Suggest a Novel Cofactor Binding Mode for Oxidoreductases. Scientific Reports (2015) 5:11674.

25. Sugio, T et al. NADH-dependent Sulfite Reductase Activity in the Periplasmic Space of Thiobacillus ferrooxidans. Bioscience, Biotechnology, and Biochemistry (1993) 57(8): 1357-1359.

26. Hallenbeck P., et al. Characterization of anaerobic sulfite reduction by Salmonella typhimurium and purification of the anaerobically induced sulfite reductase. J. Bacteriology (1989) 171(6): 3008-3015.

27. Stincone et al., The return of metabolism: biochemistry and physiology of the pentose phosphate pathway. Biol Rev Camb Philos Soc. (2015) 90(3): 927-963.

28. Cahn et al. A General Tool for Engineering the NAD/NADP Cofactor Preference of Oxidoreductases. ACS Synth. Biol. (2017) 6(2) 326-333.

29. Cui, D. et al. A computational strategy for altering an enzyme in its cofactor preference to NAD(H) and/or NADP(H). FEBS J. (2015) 282 (12):2339-51.

30. Chanique, A, et al. Protein Engineering for Nicotinamide Coenzyme Specificity in Oxidoreductases: Attempts and Challenges. Front Microbiol. (2018) 9:194.

INFORMAL SEQUENCE LISTING

>Cystathionine beta-synthase enzyme from Saccharomyces cerevisiae (strain CEN.PK113-7D)
MTKSEQQADSRHNVIDLVGNTPLIALKKLPKALGIKPQIYAKLELYNPGGSIKDRIAKSMVEEAEASGRIHPSRSTLIEPTSGN
TGIGLALIGAIKGYRTIITLPEKMSNEKVSVLKALGAEIIRTPTAAAWDSPESHIGVAKKLEKEIPGAVILDQYNNMMNPEAHYF
GIGREIQRQLEDLNLFDNLRAVVAGAGTGGTISGISKYLKEQNDKIQIVGADPFGSILAQPENLNKTDITDYKVEGIGYDFVP
QVLDRKLIDVVVYKTDDKPSFKYARQLISNEGVLVGGSSGSAFTAVVKYCEDHPELTEDDVIVAIFPDSIRSYLTKFVDDEWL
KKNNLWDDDVLARFDSSKLEASTTKYADVFGNATVKDLHLKPVVSVKETAKVTDVIKILKDNGFDQLPVLTEDGKLSGLVTL
SELLRKLSINNSNNDNTIKGKYLDFKKLNNFNDVSSYNENKSGKKKFIKFDENSKLSDLNRFFEKNSSAVITDGLKPIHIVTKM
DLLSYLA
>Cystathionine gamma-synthase enzyme from Escherichia coli
MTRKQATIAVRSGLNDDEQYGCVVPPIHLSSTYNFTGFNEPRAHDYSRRGNPTRDVVQRALAELEGGAGAVLTNTGMSAI
HLVTTVFLKPGDLLVAPHDCYGGSYRLFDSLAKRGCYRVLFVDQGDEQALRAALAEKPKLVLVESPSNPLLRVVDIAKICHL
AREVGAVSVVDNTFLSPALQNPLALGADLVLHSCTKYLNGHSDVVAGVVIAKDPDVVTELAVVVVANNIGVTGGAFDSYLLLR
GLRTLVPRMELAQRNAQAIVKYLQTQPLVKKLYHPSLPENQGHEIAARQQKGFGAMLSFELDGDEQTLRRFLGGLSLFTLA
ESLGGVESLISHAATMTHAGMAPEARAAAGISETLLRISTGIEDGEDLIADLENGFRAANKG
>Aspartate aminotransferase enzyme from Saccharomyces cerevisiae (strain CEN.PK113-7D)
MSATLFNNIELLPPDALFGIKQRYGQDQRATKVDLGIGAYRDDNGKPVVVLPSVKAAEKLIHNDSSYNHEYLGITGLPSLTSN
AAKIIFGTQSDAFQEDRVISVQSLSGTGALHISAKFFSKFFPDKLVYLSKPTWANHMAIFENQGLKTATYPYWANETKSLDLN
GFLNAIQKAPEGSIFVLHSCAHNPTGLDPTSEQWVQIVDAIASKNHIALFDTAYQGFATGDLDKDAYAVRLGVEKLSTVSPV
FVCQSFAKNAGMYGERVGCFHLALTKQAQNKTIKPAVTSQLAKIIRSEVSNPPAYGAKIVAKLLETPELTEQWHKDMVTMS
SRITKMRHALRDHLVKLGTPGNWDHIVNQCGMFSFTGLTPQMVKRLEETHAWLVASGRASIAGLNQGNVEYVAKAIDEVV
RFYATEAKL
>Feedback Deregulated (G452D) Aspartate kinase from Saccharomyces cerevisiae
MPMDFQPTSSHSNVVVVQKFGGTSVGKFPVQIVDDIVKHYSKPDGPNNNVAVVCSARSSYTKAEGTTSRLLKCCDLASQE
SEFQDIIEVIRQDHIDNADRFILNPALQAKLVDDTNKELELVKKYLNASKVLGEVSSRTVDLVMSCGEKLSCLFMTALCNDRG
CKAKYVDLSHIVPSDFSASALDNSFYTFLVQALKEKLAPFVSAKERIVPVFTGFFGLVPTGLLNGVGRGYTDLCAALIAVAVN
ADELQVWKEVDGIFTADPRKVPEARLLDSVTPEEASELTYYGSEVIHPFTMEQVIRAKIPIRIKNVQNPLGNGTIIYPDNVAKK
GESTPPHPPENLSSSFYEKRKRGATAITTKNDIFVINIHSNKKTLSHGFLAQIFTILDKYKLVVDLISTSEVHVSMALPIPDADSL
KSLRQAEEKLRILGSVDITKKLSIVSLVGKHMKQYIGIAGTMFTTLAEEGINIEMISQGANEINISCVINESDSIKALQCIHAKLLS
ERTNTSNQFEHAIDERLEQLKRLGI
>Feedback Deregulated (G378E) Homoserine dehydrogenase from Corynebacterium glutamicum
MTSASAPSFNPGKGPGSAVGIALLGFGTVGTEVMRLMTEYGDELAHRIGGPLEVRGIAVSDISKPREGVAPELLTEDAFALI
EREDVDIVVEVIGGIEYPREVVLAALKAGKSVVTANKALVAAHSAELADAAEAANVDLYFEAAVAGAIPVVGPLRRSLAGDQI
QSVMGIVNGTTNFILDAMDSTGADYADSLAEATRLGYAEADPTADVEGHDAASKAAILASIAFHTRVTADDWCEGISNISAA
DIEAAQQAGHTIKLLAICEKFTNKEGKSAISARVHPTLLPVSHPLASVNKSFNAIFVEAEAAGRLMFYGNGAGGAPTASAVLG
DVVGAARNKVHGGRAPGESTYANLPIADFGETTTRYHLDMDVEDRVGVLAELASLFSEQGISLRTIRQEERDDDARLIVVT
HSALESDLSRTVELLKAKPVVKAINSVIRLERD
>Cystathionine gamma-synthase/O-acetylhomoserine enzyme from Bacillus subtilis
MSQHVETKLAQIGNRSDEVTGTVSAPIYLSTAYRHRGIGESTGFDYVRTKNPTRQLVEDAIANLENGARGLAFSSGMAAIQT
IMALFKSGDELIVSSDLYGGTYRLFENEWKKYGLTFHYDDFSDEDCLRSKITPNTKAVFVETPTNPLMQEADIEHIARITKEH
GLLLIVDNTFYTPVLQRPLELGADIVIHSATKYLGGHNDLLAGLVVVKDERLGEEMFQHQNAIGAVLPPFDSWLLMRGMKTL
SLRMRQHQANAQELAAFLEEQEEISDVLYPGKGGMLSFRLQKEEVVVNPFLKALKTICFAESLGGVESFITYPATQTHMDIP
EEIRIANGVCNRLLRFSVGIEHAEDLKEDLKQALCQVKEGAVSFE
>Feedback Deregulated (A279T) Aspartokinase from Corynebacterium glutamicum
MALVVQKYGGSSLESAERIRNVAERIVATKKAGNDVVVVCSAMGDTTDELLELAAAVNPVPPAREMDMLLTAGERISNALV
AMAIESLGAEAQSFTGSQAGVLTTERHGNARIVDVTPGRVREALDEGKICIVAGFQGVNKETRDVTTLGRGGSDTTAVALA
AALNADVCEIYSDVDGVYTADPRIVPNAQKLEKLSFEEMLELAAVGSKILVLRSVEYARAFNVPLRVRSSYSNDPGTLIAGS
MEDIPVEEAVLTGVATDKSEAKVTVLGISDKPGEAAKVFRALADAEINIDMVLQNVSSVEDGTTDITFTCPRSDGRRAMEILK
KLQVQGNWTNVLYDDQVGKVSLVGAGMKSHPGVTAEFMEALRDVNVNIELISTSEIRISVLIREDDLDAAARALHEQFQLG
GEDEAVVYAGTGR
>Feedback Deregulated (G378S) Homoserine dehydrogenase from Corynebacterium glutamicum
MTSASAPSFNPGKGPGSAVGIALLGFGTVGTEVMRLMTEYGDELAHRIGGPLEVRGIAVSDISKPREGVAPELLTEDAFALI
EREDVDIVVEVIGGIEYPREVVLAALKAGKSVVTANKALVAAHSAELADAAEAANVDLYFEAAVAGAIPVVGPLRRSLAGDQI
QSVMGIVNGTTNFILDAMDSTGADYADSLAEATRLGYAEADPTADVEGHDAASKAAILASIAFHTRVTADDVYCEGISNISAA
DIEAAQQAGHTIKLLAICEKFTNKEGKSAISARVHPTLLPVSHPLASVNKSFNAIFVEAEAAGRLMFYGNGAGGAPTASAVLG
DVVGAARNKVHGGRAPGESTYANLPIADFGETTTRYHLDMDVEDRVGVLAELASLFSEQGISLRTIRQEERDDDARLIVVT
HSALESDLSRTVELLKAKPVVKAINSVIRLERD
>Feedback deregulated (S345F) Bifunctional aspartokinase/homoserine dehydrogenase from Escherichia coli
MRVLKFGGTSVANAERFLRVADILESNARQGQVATVLSAPAKITNHLVAMIEKTISGQDALPNISDAERIFAELLTGLAAAQP
GFPLAQLKTFVDQEFAQIKHVLHGISLLGQCPDSINAALICRGEKMSIAIMAGVLEARGHNVTVIDPVEKLLAVGHYLESTVDI
AESTRRIAASRIPADHMVLMAGFTAGNEKGELVVLGRNGSDYSAAVLAACLRADCCEIVVTDVDGVYTCDPRQVPDARLLK
SMSYQEAMELSYFGAKVLHPRTITPIAQFQIPCLIKNTGNPQAPGTLIGASRDEDELPVKGISNLNNMAMFSVSGPGMKGM
VGMAARVFAAMSRARISVVLITQSSSEYSISFCVPQSDCVRAERAMQEEFYLELKEGLLEPLAVTERLAIISVVGDGMRTLR
GISAKFFAALARANINIVAIAQGSSERSISVVVNNDDATTGVRVTHQMLFNTDQVIEVFVIGVGGVGGALLEQLKRQQSWLK
NKHIDLRVCGVANSKALLTNVHGLNLENWQEELAQAKEPFNLGRLIRLVKEYHLLNPVIVDCTSSQAVADQYADFLREGFH
VVTPNKKANTSSMDYYHQLRYAAEKSRRKFLYDINVGAGLPVIENLQNLLNAGDELMKFSGILSGSLSYIFGKLDEGMSFS
EATTLAREMGYTEPDPRDDLSGMDVARKLLILARETGRELELADIElEPVLPAEFNAEGDVAAFMANLSQLDDLFAARVAKA
RDEGKVLRYVGNIDEDGVCRVKIAEVDGNDPLFKVKNGENALAFYSHYYQPLPLVLRGYGAGNDVTAAGVFADLLRTLSW
KLGV
>Putative O-acetylhomoserine aminocarboxypropyltransferase from Corynebacterium glutamicum
MPKYDNSNADQWGFETRSIHAGQSVDAQTSARNLPIYQSTAFVFDSAEHAKQRFALEDLGPVYSRLTNPTVEALENRIASL
EGGVHAVAFSSGQAATTNAILNLAGAGDHIVTSPRLYGGTETLFLITLNRLGIDVSFVENPDDPESWQAAVQPNTKAFFGET
FANPQADVLDIPAVAEVAHRNSVPLIIDNTIATAALVRPLELGADVVVASLTKFYIGNGSGLGGVLIDGGKFDVVIVEKDGKP
VFPYFVTPDAAYHGLKYADLGAPAFGLKVRVGLLRDTGSTLSAFNAWAAVQGIDTLSLRLERHNENAIKVAEFLNNHEKVE
KVNFAGLKDSPVVYATKEKLGLKYTGSVLTFEIKGGKDEAWAFIDALKLHSNLANIGDVRSLVVHPATTTHSQSDEAGLARA
GVTQSTVRLSVGIETIDDIIADLEGGFAAI
>Cystathionine gamma-synthase from Bacillus paralicheniformis ATCC 9945a
MTEHVQTTLAQIGNRSDEITGTVNPPWFSSAYRHKGIGESTGFDYIRTKNPTRQLVEDAIAKLEGGTRGFAFSSGMAAIQTI
MALFQSGDELIVSSDLYGGTYRLFENEWKKYGLRFFYDDFSDEDCIKSKITNNTKALFVETPTNPLMQEADIQKIAQIAKEHD
LLLIVDNTFYTPVLQKPIELGADLVIHSATKYLGGHNDLLAGLVVAKGEELSEEMFQHQNAIGAVLSPFDSWLLMRGMKTLAL
RMRQHQENARELAAFLEEQEEIADVLYPGKGGMLSFRVQKEEVVVNPLLKNLKTICFAESLGGVESFITYPATQTHMDIPEDI
RIANGVCNRLLRFSVGIEHVSDLKQDLKAALEKVKGEAVPHES

What is claimed is:

1. An engineered microbial cell that expresses a heterologous cystathionine beta-synthase or a heterologous cystathionine gamma-synthase, wherein the engineered microbial cell produces cystathionine.

2. The engineered microbial cell of claim 1, wherein the engineered microbial cell expresses the heterologous cystathionine beta-synthase and the heterologous cystathionine gamma-synthase.

3. The engineered microbial cell of claim 1 or claim 2, wherein the engineered microbial cell comprises increased activity of one or more upstream pathway enzyme(s), said increased activity being increased relative to a control cell.

4. The engineered microbial cell of claim 3, wherein the engineered microbial cell comprises increased activity of one or more upstream pathway enzymes leading to cysteine.

5. The engineered microbial cell of claim 4, wherein the one or more upstream pathway enzymes leading to cysteine is/are selected from the group consisting of 3-phosphoglycerate dehydrogenase, phosphoserine transaminase, phosphoserine phosphatase, serine-O-acetyltransferase, and cysteine synthase.

6. The engineered microbial cell of any one of claims 3-5, wherein the engineered microbial cell comprises increased activity of one or more upstream pathway enzymes leading to a homoserine.

7. The engineered microbial cell of claim 6, wherein the one or more upstream pathway enzymes leading to a homoserine is/are selected from the group consisting of phosphoenolpyruvate carboxylase, pyruvate carboxylase, malate dehydrogensase, aspartate transaminase (aspartate aminotransferase), aspartate kinase (aspartokinase), aspartate-semialdehyde dehydrogenase, homoserine dehydrogenase, L-homoserine-O-acetyltransferase, and L-homoserine-O-succinyltranferase (homoserine transsuccinylase).

8. The engineered microbial cell of claim 7, wherein the one or more upstream pathway enzymes leading to homoserine is/are selected from the group consisting of pyruvate carboxylase, aspartate transaminase, and aspartate kinase.

9. The engineered microbial cell of any one of claims 3-8, wherein the engineered microbial cell comprises increased activity of one or more upstream pathway enzymes leading to homocysteine.

10. The engineered microbial cell of claim 9, wherein the one or more upstream pathway enzymes leading to homocysteine is/are selected from the group consisting of sulfate adenyltransferase (ATP sulfurylase), adenyl-sulfate kinase (APS kinase), phosphoadenosine phosphosulfate (PAPS) reductase, sulfite reductase, and homocysteine synthase.

11. The engineered microbial cell of claim 10, wherein the one or more upstream pathway enzymes leading to homocysteine comprises sulfite reductase.

12. The engineered microbial cell of any one of claims 3-11, wherein the engineered microbial cell comprises increased activity of one or more upstream pathway enzymes leading to serine.

13. The engineered microbial cell of claim 12, wherein the one or more upstream pathway enzymes leading to serine is/are selected from the group consisting of 3-phosphoglycerate dehydrogenase, phosphoserine transaminase, and phosphoserine phosphatase

14. The engineered microbial cell of any one of claims 1-13, wherein the activity of the one or more upstream pathway enzymes is increased by introducing one or more genes encoding the one or more upstream pathway enzymes.

15. The engineered microbial cell of claim 14, wherein at least two genes encoding the same enzyme are introduced.

16. The engineered microbial cell of any one of claims 3-15, wherein the activity of the one or more upstream pathway enzymes is increased by introducing one or more feedback-deregulated enzyme(s).

17. The engineered microbial cell of claim 16, where the one or more feedback-deregulated enzyme (s) is/are selected from the group consisting of a feedback-deregulated aspartate kinase, a feedback-deregulated homoserine dehydrogenase, a feedback-deregulated aspartate-semialdehyde dehydrogenase, a feedback-deregulated L-homoserine-O-succinyltranferase, a feedback-deregulated phoshoenolpyruvate carboxylase, and a feedback-deregulated pyruvate carboxylase.

18. The engineered microbial cell of claim 17, where the one or more feedback-deregulated enzyme(s) is/are selected from the group consisting of:
(a) a feedback-deregulated Saccharomyces cerevisiae aspartate kinase (EC 2.7.2.4) comprising the amino acid substitution E250K or M318I;
(b) a feedback-deregulated homoserine dehydrogenase (EC 1.1.1.3) comprising (i) the amino acid substitutions V104I, T116I, and G148A; or (ii) the amino acid substitutions A429L, K430S, P431L, V432L, V433L, K434R, A435Q, I436S, N437T, and S438V, and a deletion of amino acids 439-445;
(c) a feedback-deregulated aspartate-semialdehyde dehydrogenase (EC 1.2.1.11) comprising the amino acid substitutions D66G, S202F, R234H, D272E, and K285E;
(d) a feedback-deregulated L-homoserine-O-succinyltranferase (EC 2.3.1.46) comprising the amino acid substitution R27C or I296S;
(e) a feedback-deregulated phosphoenol pyruvate carboxylase (EC 4.1.1.31) comprising the amino acid substitution N917G or D299N; and
(f) a feedback-deregulated pyruvate carboxylase (EC 6.4.1.1) comprising the amino acid substitution P458S.

19. The engineered microbial cell of claim 18, wherein the one or more feedback-deregulated enzyme(s) comprise a feedback-deregulated Saccharomyces cerevisiae aspartate kinase (EC 2.7.2.4) comprising the amino acid substitution E250K or M3181.

20. The engineered microbial cell of any one of claims 1-19, wherein the engineered microbial cell comprises reduced activity of one or more enzyme(s) that consume one or more upstream pathway precursors, said reduced activity being reduced relative to a control cell.

21. The engineered microbial cell of claim 20, wherein the one or more enzyme(s) that consume one or more upstream pathway precursors is/are selected from the group consisting of methionine synthase, homoserine kinase, threonine synthase, catabolic serine deaminase, glutathione synthase, and L-cysteine desulfhydrase.

22. The engineered microbial cell of any one of claims 1-21, wherein the engineered microbial cell comprises reduced activity of one or more enzyme(s) that consume cystathionine, said reduced activity being reduced relative to a control cell.

23. The engineered microbial cell of claim 22, wherein the one or more enzyme(s) that consume cystathionine are selected from cystathionine beta-lyase and cystathionine gamma-lyase.

24. The engineered microbial cell of any one of claims 20-23, wherein the reduced activity is achieved by one or more means selected from the group consisting of gene deletion, gene disruption, altering regulation of a gene, and replacing a native promoter with a less active promoter.

25. The engineered microbial cell of any one of claims 1-24, wherein the engineered microbial cell comprises increased activity of an amino acid exporter that is capable of exporting cystathionine, said increased activity being increased relative to a control cell.

26. The engineered microbial cell of any of claims 1-25, wherein the engineered microbial cell comprises altered cofactor specificity of one or more upstream pathway enzyme(s) from the reduced form of nicotinamide adenine dinucleotide phosphate (NADPH) to the reduced from of nicotinamide adenine dinucleotide (NADH).

27. The engineered microbial cell of claim 26, wherein the one or more upstream pathway enzyme(s) whose cofactor specificity is altered is/are selected from the group consisting of aspartate semi-aldehyde dehydrogenase, homoserine dehydrogenase, and glyceraldehyde 3-phosphate dehydrogenase (GAPDH).

28. The engineered microbial cell of any one of claims 1-27, wherein the engineered microbial cell is a bacterial cell.

29. The engineered microbial cell of claim 28, wherein the bacterial cell is a Corynebacteria glutamicum cell.

30. The engineered microbial cell of claim 29, wherein the engineered microbial cell comprises a heterologous cystathionine beta-synthase having at least 70% amino acid sequence identity with a Saccharomyces cerevisiae cystathionine beta-synthase.

31. The engineered microbial cell of claim 30, wherein the engineered microbial cell additionally comprises a heterologous cystathionine gamma-synthase having at least 70% amino acid sequence identity with an Escherichia coli cystathionine gamma-synthase.

32. The engineered microbial cell of claim 30 or claim 31, wherein the engineered microbial cell additionally comprises a heterologous aspartate aminotransferase having at least 70% amino acid sequence identity with a Saccharomyces cerevisiae aspartate aminotransferase.

33. The engineered microbial cell of claim 28, wherein the bacterial cell is a Bacillus subtilis cell.

34. The engineered microbial cell of claim 33, wherein the engineered microbial cell comprises a heterologous cystathionine beta-synthase having at least 70% amino acid sequence identity with a Saccharomyces cerevisiae cystathionine beta-synthase.

35. The engineered microbial cell of claim 34, wherein the engineered microbial cell additionally comprises a heterologous cystathionine gamma-synthase having at least 70% amino acid sequence identity with a Bacillus paralicheniformis cystathionine gamma-synthase.

36. The engineered microbial cell of claim 34 or claim 35, wherein the engineered microbial cell additionally comprises a feedback-deregulated aspartokinase having at least 70% amino acid sequence identity with a feedback-deregulated Saccharomyces cerevisiae aspartokinase.

37. The engineered microbial cell of any one of claims 1-27, wherein the engineered microbial cell comprises a yeast cell.

38. The engineered microbial cell of claim 37, wherein the yeast cell is a Saccharomyces cerevisiae cell.

39. The engineered microbial cell of claim 38, wherein the engineered microbial cell comprises a heterologous cystathionine beta-synthase having at least 70% amino acid sequence identity with a Saccharomyces cerevisiae cystathionine beta-synthase.

40. The engineered microbial cell of claim 39, wherein the engineered microbial cell additionally comprises a heterologous cystathionine gamma-synthase having at least 70% amino acid sequence identity with an Escherichia coli cystathionine gamma-synthase.

41. The engineered microbial cell of claim 39 or 40, wherein the engineered microbial cell additionally comprises a feedback-deregulated aspartokinase having at least 70% amino acid sequence identity with a feedback-deregulated Saccharomyces cerevisiae aspartokinase.

42. The engineered microbial cell of claim 37, wherein the yeast cell is a Yarrowia lipolytica cell.

43. The engineered microbial cell of claim 42, wherein the engineered microbial cell comprises a heterologous cystathionine beta-synthase having at least 70% amino acid sequence identity with a Saccharomyces cerevisiae cystathionine beta-synthase.

44. The engineered microbial cell of claim 43, wherein the engineered microbial cell additionally comprises a heterologous cystathionine gamma-synthase having at least 70% amino acid sequence identity with a Bacillus paralicheniformis cystathionine gamma-synthase.

45. The engineered microbial cell of claim 43 or claim 44, wherein the engineered microbial cell additionally comprises a feedback-deregulated aspartokinase having at least 70% amino acid sequence identity with a feedback-deregulated Saccharomyces cerevisiae aspartokinase.

46. The engineered microbial cell of any one of claims 1-45, wherein, when cultured, the engineered microbial cell produces cystathionine at a level at least 50 μg/L of culture medium.

47. The engineered microbial cell of claim 46, wherein, when cultured, the engineered microbial cell produces cystathionine at a level at least 1 mg/L of culture medium.

48. A culture of engineered microbial cells according to any one of claims 1-47, optionally wherein the culture comprises cystathionine at a level at least 4 mg/L of culture medium.

49. A method of culturing engineered microbial cells according to any one of claims 1-47, the method comprising culturing the cells under conditions suitable for producing cystathionine, optionally wherein the method additionally comprises recovering cystathionine from the culture.


SEQUENCE LISTING (11 sequences)

SEQ ID NO: 1 - protein, 507 aa, Saccharomyces cerevisiae (strain CEN.PK113-7D)
MTKSEQQADSRHNVIDLVGNTPLIALKKLPKALGIKPQIYAKLELYNPGGSIKDRIAKSMVEEAEASGRIHPSRSTLIEPTSGNTGIGLALIGAIKGYRTIITLPEKMSNEKVSVLKALGAEIIRTPTAAAWDSPESHIGVAKKLEKEIPGAVILDQYNNMMNPEAHYFGTGREIQRQLEDLNLFDNLRAVVAGAGTGGTISGISKYLKEQNDKIQIVGADPFGSILAQPENLNKTDITDYKVEGIGYDFVPQVLDRKLIDVWYKTDDKPSFKYARQLISNEGVLVGGSSGSAFTAVVKYCEDHPELTEDDVIVAIFPDSIRSYLTKFVDDEWLKKNNLWDDDVLARFDSSKLEASTTKYADVFGNATVKDLHLKPVVSVKETAKVTDVIKILKDNGFDQLPVLTEDGKLSGLVTLSELLRKLSINNSNNDNTIKGKYLDFKKLNNFNDVSSYNENKSGKKKFIKFDENSKLSDLNRFFEKNSSAVITDGLKPIHIVTKMDLLSYLA

SEQ ID NO: 2 - protein, 386 aa, Escherichia coli
MTRKQATIAVRSGLNDDEQYGCVVPPIHLSSTYNFTGFNEPRAHDYSRRGNPTRDVVQRALAELEGGAGAVLTNTGMSAIHLVTTVFLKPGDLLVAPHDCYGGSYRLFDSLAKRGCYRVLFVDQGDEQALRAALAEKPKLVLVESPSNPLLRVVDIAKICHLAREVGAVSVVDNTFLSPALQNPLALGADLVLHSCTKYLNGHSDVVAGVVIAKDPDVVTELAWWANNIGVTGGAFDSYLLLRGLRTLVPRMELAQRNAQAIVKYLQTQPLVKKLYHPSLPENQGHEIAARQQKGFGAMLSFELDGDEQTLRRFLGGLSLFTLAESLGGVESLISHAATMTHAGMAPEARAAAGISETLLRISTGIEDGEDLIADLENGFRAANKG

SEQ ID NO: 3 - protein, 418 aa, Saccharomyces cerevisiae (strain CEN.PK113-7D)
MSATLFNNIELLPPDALFGIKQRYGQDQRATKVDLGIGAYRDDNGKPWVLPSVKAAEKLIHNDSSYNHEYLGITGLPSLTSNAAKIIFGTQSDAFQEDRVISVQSLSGTGALHISAKFFSKFFPDKLVYLSKPTWANHMAIFENQGLKTATYPYWANETKSLDLNGFLNAIQKAPEGSIFVLHSCAHNPTGLDPTSEQWVQIVDAIASKNHIALFDTAYQGFATGDLDKDAYAVRLGVEKLSTVSPVFVCQSFAKNAGMYGERVGCFHLALTKQAQNKTIKPAVTSQLAKIIRSEVSNPPAYGAKIVAKLLETPELTEQWHKDMVTMSSRITKMRHALRDHLVKLGTPGNWDHIVNQCGMFSFTGLTPQMVKRLEETHAVYLVASGRASIAGLNQGNVEYVAKAIDEVVRFYATEAKL

SEQ ID NO: 4 - protein, 527 aa, Artificial
  Mutated sequence
MPMDFQPTSSHSNWVVQKFGGTSVGKFPVQIVDDIVKHYSKPDGPNNNVAVVCSARSSYTKAEGTTSRLLKCCDLASQESEFQDIIEVIRQDHIDNADRFILNPALQAKLVDDTNKELELVKKYLNASKVLGEVSSRTVDLVMSCGEKLSCLFMTALCNDRGCKAKYVDLSHIVPSDFSASALDNSFYTFLVQALKEKLAPFVSAKERIVPVFTGFFGLVPTGLLNGVGRGYTDLCAALIAVAVNADELQVWKEVDGIFTADPRKVPEARLLDSVTPEEASELTYYGSEVIHPFTMEQVIRAKIPIRIKNVQNPLGNGTIIYPDNVAKKGESTPPHPPENLSSSFYEKRKRGATAITTKNDIFVINIHSNKKTLSHGFLAQIFTILDKYKLVVDLISTSEVHVSMALPIPDADSLKSLRQAEEKLRILGSVDITKKLSIVSLVGKHMKQYIGIAGTMFTTLAEEGINIEMISQGANEINISCVINESDSIKALQCIHAKLLSERTNTSNQFEHAIDERLEQLKRLGI

SEQ ID NO: 5 - protein, 445 aa, Artificial
  Mutated sequence
MTSASAPSFNPGKGPGSAVGIALLGFGTVGTEVMRLMTEYGDELAHRIGGPLEVRGIAVSDISKPREGVAPELLTEDAFALIEREDVDIVVEVIGGIEYPREVVLAALKAGKSVVTANKALVAAHSAELADAAEAANVDLYFEAAVAGAIPVVGPLRRSLAGDQIQSVMGIVNGTTNFILDAMDSTGADYADSLAEATRLGYAEADPTADVEGHDAASKAAILASIAFHTRVTADDVYCEGISNISAADIEAAQQAGHTIKLLAICEKFTNKEGKSAISARVHPTLLPVSHPLASVNKSFNAIFVEAEAAGRLMFYGNGAGGAPTASAVLGDVVGAARNKVHGGRAPGESTYANLPIADFGETTTRYHLDMDVEDRVGVLAELASLFSEQGISLRTIRQEERDDDARLIVVTHSALESDLSRTVELLKAKPVVKAINSVIRLERD

SEQ ID NO: 6 - protein, 373 aa, Bacillus subtilis
MSQHVETKLAQIGNRSDEVTGTVSAPIYLSTAYRHRGIGESTGFDYVRTKNPTRQLVEDAIANLENGARGLAFSSGMAAIQTIMALFKSGDELIVSSDLYGGTYRLFENEWKKYGLTFHYDDFSDEDCLRSKITPNTKAVFVETPTNPLMQEADIEHIARITKEHGLLLIVDNTFYTPVLQRPLELGADIVIHSATKYLGGHNDLLAGLVVVKDERLGEEMFQHQNAIGAVLPPFDSWLLMRGMKTLSLRMRQHQANAQELAAFLEEQEEISDVLYPGKGGMLSFRLQKEEWVNPFLKALKTICFAESLGGVESFITYPATQTHMDIPEEIRIANGVCNRLLRFSVGIEHAEDLKEDLKQALCQVKEGAVSFE

SEQ ID NO: 7 - protein, 421 aa, Artificial
  Mutated sequence
MALVVQKYGGSSLESAERIRNVAERIVATKKAGNDVVVVCSAMGDTTDELLELAAAVNPVPPAREMDMLLTAGERISNALVAMAIESLGAEAQSFTGSQAGVLTTERHGNARIVDVTPGRVREALDEGKICIVAGFQGVNKETRDVTTLGRGGSDTTAVALAAALNADVCEIYSDVDGVYTADPRIVPNAQKLEKLSFEEMLELAAVGSKILVLRSVEYARAFNVPLRVRSSYSNDPGTLIAGSMEDIPVEEAVLTGVATDKSEAKVTVLGISDKPGEAAKVFRALADAEINIDMVLQNVSSVEDGTTDITFTCPRSDGRRAMEILKKLQVQGNWTNVLYDDQVGKVSLVGAGMKSHPGVTAEFMEALRDVNVNIELISTSEIRISVLIREDDLDAAARALHEQFQLGGEDEAVVYAGTGR

SEQ ID NO: 8 - protein, 445 aa, Artificial; identical to SEQ ID NO: 5
  Mutated sequence

SEQ ID NO: 9 - protein, 820 aa, Artificial
  Mutated sequence
MRVLKFGGTSVANAERFLRVADILESNARQGQVATVLSAPAKITNHLVAMIEKTISGQDALPNISDAERIFAELLTGLAAAQPGFPLAQLKTFVDQEFAQIKHVLHGISLLGQCPDSINAALICRGEKMSIAIMAGVLEARGHNVTVIDPVEKLLAVGHYLESTVDIAESTRRIAASRIPADHMVLMAGFTAGNEKGELVVLGRNGSDYSAAVLAACLRADCCEIWTDVDGVYTCDPRQVPDARLLKSMSYQEAMELSYFGAKVLHPRTITPIAQFQIPCLIKNTGNPQAPGTLIGASRDEDELPVKGISNLNNMAMFSVSGPGMKGMVGMAARVFAAMSRARISVVLITQSSSEYSISFCVPQSDCVRAERAMQEEFYLELKEGLLEPLAVTERLAIISVVGDGMRTLRGISAKFFAALARANINIVAIAQGSSERSISVVVNNDDATTGVRVTHQMLFNTDQVIEVFVIGVGGVGGALLEQLKRQQSWLKNKHIDLRVCGVANSKALLTNVHGLNLENWQEELAQAKEPFNLGRLIRLVKEYHLLNPVIVDCTSSQAVADQYADFLREGFHVVTPNKKANTSSMDYYHQLRYAAEKSRRKFLYDTNVGAGLPVIENLQNLLNAGDELMKFSGILSGSLSYIFGKLDEGMSFSEATTLAREMGYTEPDPRDDLSGMDVARKLLILARETGRELELADIEIEPVLPAEFNAEGDVAAFMANLSQLDDLFAARVAKARDEGKVLRYVGNIDEDGVCRVKIAEVDGNDPLFKVKNGENALAFYSHYYQPLPLVLRGYGAGNDVTAAGVFADLLRTLSWKLGV

SEQ ID NO: 10 - protein, 437 aa, Corynebacterium glutamicum
MPKYDNSNADQWGFETRSIHAGQSVDAQTSARNLPIYQSTAFVFDSAEHAKQRFALEDLGPVYSRLTNPTVEALENRIASLEGGVHAVAFSSGQAATTNAILNLAGAGDHIVTSPRLYGGTETLFLITLNRLGIDVSFVENPDDPESWQAAVQPNTKAFFGETFANPQADVLDIPAVAEVAHRNSVPLIIDNTIATAALVRPLELGADVVVASLTKFYTGNGSGLGGVLIDGGKFDWTVEKDGKPVFPYFVTPDAAYHGLKYADLGAPAFGLKVRVGLLRDTGSTLSAFNAWAAVQGIDTLSLRLERHNENAIKVAEFLNNHEKVEKVNFAGLKDSPWYATKEKLGLKYTGSVLTFEIKGGKDEAWAFIDALKLHSNLANIGDVRSLVVHPATTTHSQSDEAGLARAGVTQSTVRLSVGIETIDDIIADLEGGFAAI

SEQ ID NO: 11 - protein, 374 aa, Bacillus paralicheniformis ATCC 9945a
MTEHVQTTLAQIGNRSDEITGTVNPPVYFSSAYRHKGIGESTGFDYIRTKNPTRQLVEDAIAKLEGGTRGFAFSSGMAAIQTIMALFQSGDELIVSSDLYGGTYRLFENEWKKYGLRFFYDDFSDEDCIKSKITNNTKALFVETPTNPLMQEADIQKIAQIAKEHDLLLIVDNTFYTPVLQKPIELGADLVIHSATKYLGGHNDLLAGLVVAKGEELSEEMFQHQNAIGAVLSPFDSWLLMRGMKTLALRMRQHQENARELAAFLEEQEEIADVLYPGKGGMLSFRVQKEEWVNPLLKNLKTICFAESLGGVESFITYPATQTHMDIPEDIRIANGVCNRLLRFSVGIEHVSDLKQDLKAALEKVKGEAVPHES